SEQUENCE LISTING (125 sequences)

SEQ ID NO: 1 - DNA, 81 bp, Homo sapiens
ttgctcataggcatctccatcgcgagcctgtgcctggtggtggcgcttttggcgctcctctgccacctgcgcaagaagcag

SEQ ID NO: 2 - protein, 27 aa, Homo sapiens
LLIGISIASLCLVVALLALLCHLRKKQ

SEQ ID NO: 3 - DNA, 332 bp, Saccharomyces cerevisiae
acaagttcttacacatcttctacttacactgcaaaaatttcttctacctccgctgctgctacttcttctgctccagcagcgctgccagcagccaataaaacttcatctcacaataaaaaagagtagcaattgcgtgcggtgttgctatcccattaggcgttatcctagtagctctcatttgcttcctaatattctggagacgcagaagggaaaatccagacgatgaaaacttaccgcatgctattagtggacctgatttgaataatcctgcaaataaaccaaatcaagaaaacgctacacctttgaacaacccctttgatgatgatgcttcc

SEQ ID NO: 4 - protein, 111 aa, Saccharomyces cerevisiae
TSSYTSSTYTAKISSTSAAATSSAPAALPAANKTSSHNKKAVAIACGVAIPLGVILVALICFLIFWRRRRENPDDENLPHAISGPDLNNPANKPNQENATPLNNPFDDDAS

SEQ ID NO: 5 - DNA, 225 bp, Saccharomyces cerevisiae
caactcaacctgaacttcgatgtagatcactcagattcatctttagttgacaaattcggcattaagccagaaatccatcatatatttcatgccgagccaaagagagttgccaaaccaattgctgtgatttttgttttaattatcttcattaccattttatctttaatagtcacctggttgaattcttgtgccgccgcattcaataacatcccgaccggtgtcact

SEQ ID NO: 6 - protein, 75 aa, Saccharomyces cerevisiae
QLNLNFDVDHSDSSLVDKFGIKPEIHHIFHAEPKRVAKPIAVIFVLIIFITILSLIVTWLNSCAAAFNNIPTGVT

SEQ ID NO: 7 - protein, 40 aa, Saccharomyces cerevisiae
EKSTNSSSSATSKNAGAAMDMGFFSAGVGAAIAGAAAMLL

SEQ ID NO: 8 - protein, 43 aa, Saccharomyces cerevisiae
SLLKSAASATSSSQSSSKSKGAAGIIEIPLIFRALAELYNLVL

SEQ ID NO: 9 - protein, 40 aa, Saccharomyces cerevisiae
SSGASSSSSKSSKGNAAIMAPIGQTTPLVGLLTAIIMSIM

SEQ ID NO: 10 - protein, 44 aa, Saccharomyces cerevisiae
AQANVSASASSSSSSSKKSKGAAPELVPATSFMGVVAAVGVALL

SEQ ID NO: 11 - protein, 40 aa, Saccharomyces cerevisiae
GPGEKARKNNAAPGPSNFNSIKLFGVTAGSAAVAGALLLL

SEQ ID NO: 12 - protein, 39 aa, Saccharomyces cerevisiae
SSTGMLSPTSSSSTRKENGGHNLNPPFFARFITAIFHHI

SEQ ID NO: 13 - protein, 40 aa, Saccharomyces cerevisiae
SSFSSSGGSSESTTKKQNAGYKYRSSFSFSLLSFISYFLL

SEQ ID NO: 14 - protein, 40 aa, Saccharomyces cerevisiae
YKSTVNGKVASVMSNSTNGATAGTHIAYGAGAFAVGALLL

SEQ ID NO: 15 - protein, 40 aa, Saccharomyces cerevisiae
SGNLTTSTASATSTSSKRNVGDHIVPSLPLTLISLLFAFI

SEQ ID NO: 16 - protein, 43 aa, Saccharomyces cerevisiae
GKNGAKSQGSSKKMENSAPKNIFIDAFKMSVYAVFTVLFSIIF

SEQ ID NO: 17 - protein, 40 aa, Saccharomyces cerevisiae
TGSSSASSSSKSKGVGNIVNVSFSQSGYLALFAGLISALL

SEQ ID NO: 18 - protein, 40 aa, Saccharomyces cerevisiae
ASGSSTHKKNAGNALVNYSNLNTNTFIGVLSVISAVFGLI

SEQ ID NO: 19 - protein, 40 aa, Saccharomyces cerevisiae
EDADEDKDDLKRKHRNSASISGPLLPLGLCLLFFTFSLFF

SEQ ID NO: 20 - DNA, 254 bp, Saccharomyces cerevisiae
atcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagct

SEQ ID NO: 21 - DNA, 288 bp, Saccharomyces cerevisiae
gatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaatt

SEQ ID NO: 22 - DNA, 54 bp, Saccharomyces cerevisiae
atgcagttacttcgctgtttttcaatattttctgttattgcttcagttttagca

SEQ ID NO: 23 - protein, 18 aa, Saccharomyces cerevisiae
MQLLRCFSIFSVIASVLA

SEQ ID NO: 24 - DNA, 267 bp, Saccharomyces cerevisiae
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctttggataaaagagaggctgaagct

SEQ ID NO: 25 - protein, 89 aa, Saccharomyces cerevisiae
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 26 - DNA, 43 bp, Artificial Sequence
  oligonucleotide primer
caatattttctgttattgctagcgttttagcacaggaactgac

SEQ ID NO: 27 - DNA, 43 bp, Artificial Sequence
  oligonucleotide primer
gtcagttcctgtgctaaaacgctagcaataacagaaaatattg

SEQ ID NO: 28 - DNA, 42 bp, Artificial Sequence
  oligonucleotide primer
gttattgctagcgttttagcagacatccagatgacccagtct

SEQ ID NO: 29 - DNA, 41 bp, Artificial Sequence
  oligonucleotide primer
ttgcggccgctatactagtgacatcgattcactaacactct

SEQ ID NO: 30 - DNA, 39 bp, Artificial Sequence
  oligonucleotide primer
aactagtaaaagaatgagatttccttcaatttttactgc

SEQ ID NO: 31 - DNA, 45 bp, Artificial Sequence
  oligonucleotide primer
ggtaataagcttgaattcagcttcagcctctcttttctcgagaga

SEQ ID NO: 32 - DNA, 39 bp, Artificial Sequence
  oligonucleotide primer
gtatctctcgagaaaagacaaatgcagctggtgcagtct

SEQ ID NO: 33 - DNA, 75 bp, Artificial Sequence
  oligonucleotide primer
agcgccaccaccaggcacaggctcgcgatggagatgcctatgagcaagcctcctttacccggagacaggcttaag

SEQ ID NO: 34 - DNA, 70 bp, Artificial Sequence
  oligonucleotide primer
tagcggccgctcactgcttcttgcgcaggtggcagaggagcgccaaaagcgccaccaccaggcacaggct

SEQ ID NO: 35 - DNA, 60 bp, Artificial Sequence
  oligonucleotide primer
aagagcttaagcctgtctccgggtaaaggcggtggcggaagcgccaaaagctcttttatc

SEQ ID NO: 36 - DNA, 44 bp, Artificial Sequence
  oligonucleotide primer
agcgggtttgcggccgctcattagaatagcaggtacgacaaaag

SEQ ID NO: 37 - DNA, 53 bp, Artificial Sequence
  oligonucleotide primer
gaagagcttaagcctgtctccgggtaaacaactcaacctgaacttcgatgtag

SEQ ID NO: 38 - DNA, 46 bp, Artificial Sequence
  oligonucleotide primer
cgggtttgcggccgctcattaagtgacaccggtcgggatgttattg

SEQ ID NO: 39 - DNA, 51 bp, Artificial Sequence
  oligonucleotide primer
gaagagcttaagcctgtctccgggtaaaacaagttcttacacatcttctac

SEQ ID NO: 40 - DNA, 46 bp, Artificial Sequence
  oligonucleotide primer
cgggtttgcggccgctcattaggaagcatcatcatcaaaggggttg

SEQ ID NO: 41 - DNA, 45 bp, Artificial Sequence
  oligonucleotide primer
ggtatctctcgagaaaagagaggtgcagctggtggagtctggggg

SEQ ID NO: 42 - DNA, 45 bp, Artificial Sequence
  oligonucleotide primer
agacaggcttaagcaagatttgggctcaactctcttgtccacctt

SEQ ID NO: 43 - DNA, 44 bp, Artificial Sequence
  oligonucleotide primer
gttattgctagcgttttagcacagtctgtgctgacgcagccgcc

SEQ ID NO: 44 - DNA, 48 bp, Artificial Sequence
  oligonucleotide primer
tcctttacccggagacaggcttaaggaacattctgtaggggccactgt

SEQ ID NO: 45 - DNA, 24 bp, Artificial Sequence
  oligonucleotide primer
ttaagcctgtctccgggtaaagga

SEQ ID NO: 46 - DNA, 19 bp, Artificial Sequence
  oligonucleotide primer
gtcacgcttacattcacgc

SEQ ID NO: 47 - DNA, 44 bp, Artificial Sequence
  oligonucleotide primer
gggtttgcggccgctcattaacaagatttgggctcaactctctt

SEQ ID NO: 48 - DNA, 60 bp, Artificial Sequence
  oligonucleotide primer
tctctcgagaaaagagactacaaagatgacgatgacaaagaggtgcagctggtggagtct

SEQ ID NO: 49 - DNA, 51 bp, Artificial Sequence
  oligonucleotide primer
ggagccgccgccgccagaaccaccaccacctgaggagacggtgaccatggt

SEQ ID NO: 50 - DNA, 54 bp, Artificial Sequence
  oligonucleotide primer
tctggcggcggcggctccggtggtggtggttctgccatccagttgactcagtct

SEQ ID NO: 51 - DNA, 36 bp, Artificial Sequence
  oligonucleotide primer
gacaggcttaagcctttgatctccagcttggtccct

SEQ ID NO: 52 - DNA, 59 bp, Artificial Sequence
  oligonucleotide primer
tctctcgagaaaagagactacaaagatgacgatgacaaacaggtgcagctgcaggagtc

SEQ ID NO: 53 - DNA, 51 bp, Artificial Sequence
  oligonucleotide primer
ggagccgccgccgccagaaccaccaccacctgaggagacggtgaccagggt

SEQ ID NO: 54 - DNA, 54 bp, Artificial Sequence
  oligonucleotide primer
tctggcggcggcggctccggtggtggtggttctcagtctgtgttgacgcagccg

SEQ ID NO: 55 - DNA, 36 bp, Artificial Sequence
  oligonucleotide primer
gacaggcttaagcctaggacggtcagcttggtccct

SEQ ID NO: 56 - DNA, 34 bp, Artificial Sequence
  oligonucleotide primer
tttggggtcgactttgatctccagcttggtccct

SEQ ID NO: 57 - DNA, 30 bp, Artificial Sequence
  oligonucleotide primer
gtcgaccccaaatctagtgacaaaactcac

SEQ ID NO: 58 - DNA, 41 bp, Artificial Sequence
  oligonucleotide primer
ggcccttggtcgacgctgaggagacggtgaccatggtgccc

SEQ ID NO: 59 - DNA, 1350 bp, Artificial Sequence
  oligonucleotide primer
caaatgcagctggtgcagtctgggcctgaggtgaagaagcctgggacctcagtgaaggtctcctgcaaggcttctggattcacctttgacgattactccatgaactgggtgcgacaggctcgtggacaacgccttgagtggataggatttattagaaacaaagctaatgactacacaacagagtacgctgactctgtgaagggtagagtcaccattaccagggacatgtccacgagcacagcctacatggagctgagcagcctgagatccgaggacacggccgtgtattactgtgcgagataccctaggcatcatgctatggactcctggggccaaggaacctcggtcaccgtctcctcagcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaa

SEQ ID NO: 60 - DNA, 642 bp, Artificial Sequence
  oligonucleotide primer
gacatccagatgacccagtctccatcctccctgtctgcatctgtaggagacagagtcaccatcacttgcagggccagccaaagtattagcaacaacctacactggtatcagcagaaaccagggaaagcccctaagctcctgatcaagtatgccttccagtccatctctggggtcccatcaaggttcagtggaagtggatctgggacagattttactttcaccatcagcagcctgcagcctgaagattttgcaacatattactgtcaacaggccaacagctggccgctcacgttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 61 - DNA, 1326 bp, Artificial Sequence
  oligonucleotide primer
cagtctgtgttgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacaacagcctgagtggttcggtggttttcggcggagggaccaagctgaccgtcctagcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaa

SEQ ID NO: 62 - DNA, 654 bp, Artificial Sequence
  oligonucleotide primer
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacaacagcctgagtggttcggtggttttcggcggagggaccaagctgaccgtcctaggtcagcccaaggcggcgccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 63 - DNA, 960 bp, Artificial Sequence
  oligonucleotide primer
agcgccaaaagctcttttatctcaaccactactactgatttaacaagtataaacactagtgcgtattccactggatccatttccacagtagaaacaggcaatcgaactacatcagaagtgatcagccatgtggtgactaccagcacaaaactgtctccaactgctactaccagcctgacaattgcacaaaccagtatctattctactgactcaaatatcacagtaggaacagatattcacaccacatcagaagtgattagtgatgtggaaaccattagcagagaaacagcttcgaccgttgtagccgctccaacctcaacaactggatggacaggcgctatgaatacttacatctcgcaatttacatcctcttctttcgcaacaatcaacagcacaccaataatctcttcatcagcagtatttgaaacctcagatgcttcaattgtcaatgtgcacactgaaaatatcacgaatactgctgctgttccatctgaagagcccacttttgtaaatgccacgagaaactccttaaattccttctgcagcagcaaacagccatccagtccctcatcttatacgtcttccccactcgtatcgtccctctccgtaagcaaaacattactaagcaccagttttacgccttctgtgccaacatctaatacatatatcaaaacgaaaaatacgggttactttgagcacacggctttgacaacatcttcagttggccttaattcttttagtgaaacagcagtctcatctcagggaacgaaaattgacacctttttagtgtcatccttgatcgcatatccttcttctgcatcaggaagccaattgtccggtatccaacagaatttcacatcaacttctctcatgatttcaacctatgaaggtaaagcgtctatatttttctcagctgagctcggttcgatcatttttctgcttttgtcgtacctgctattc

SEQ ID NO: 64 - DNA, 669 bp, Artificial Sequence
  oligonucleotide primer
gaggtgcagctggtggagtctgggggaggtgtggtacggcctggggggtccctgagactctcctgtgcagcctctgggttcaccgtcagtgattactccatgaactgggtccgccaggctccagggaagggcctggagtggattgggtttattagaaacaaagctaatgcctacacaacagagtacagtgcatctgtgaagggtagattcaccatctcaagagatgattcaaaaaacacgctgtatctgcaaatgaacagcctgaaaaccgaggacacagccgtgtattactgtaccacataccctaggtatcatgctatggactcctggggccagggcaccatggtcaccgtctcctcagcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgc

SEQ ID NO: 65 - DNA, 642 bp, Artificial Sequence
  oligonucleotide primer
gccatccagttgactcagtctccatcctccctgtctgcatctgtaggagacagagtcaccatcacttgcagggccagccaaagtattagcaacaacctacactggtacctgcagaagccagggcagtctccacagctcctgatctattatggcttccagtccatctctggggtcccatcaaggttcagtggcagtggatctgggacagatttcactctcaccatcagcagtctgcaacctgaagattttgcaacttactactgtcaacaggccaacagctggccgctcacgttcggcggagggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 66 - DNA, 726 bp, Artificial Sequence
  oligonucleotide primer
gaggtgcagctggtggagtctgggggaggtgtggtacggcctggggggtccctgagactctcctgtgcagcctctgggttcaccgtcagtgattactccatgaactgggtccgccaggctccagggaagggcctggagtggattgggtttattagaaacaaagctaatgcctacacaacagagtacagtgcatctgtgaagggtagattcaccatctcaagagatgattcaaaaaacacgctgtatctgcaaatgaacagcctgaaaaccgaggacacagccgtgtattactgtaccacataccctaggtatcatgctatggactcctggggccagggcaccatggtcaccgtctcctcaggtggtggtggttctggcggcggcggctccggtggtggtggttctgccatccagttgactcagtctccatcctccctgtctgcatctgtaggagacagagtcaccatcacttgcagggccagccaaagtattagcaacaacctacactggtacctgcagaagccagggcagtctccacagctcctgatctattatggcttccagtccatctctggggtcccatcaaggttcagtggcagtggatctgggacagatttcactctcaccatcagcagtctgcaacctgaagattttgcaacttactactgtcaacaggccaacagctggccgctcacgttcggcggagggaccaagctggagatcaaa

SEQ ID NO: 67 - DNA, 1425 bp, Artificial Sequence
  oligonucleotide primer
gaggtgcagctggtggagtctgggggaggtgtggtacggcctggggggtccctgagactctcctgtgcagcctctgggttcaccgtcagtgattactccatgaactgggtccgccaggctccagggaagggcctggagtggattgggtttattagaaacaaagctaatgcctacacaacagagtacagtgcatctgtgaagggtagattcaccatctcaagagatgattcaaaaaacacgctgtatctgcaaatgaacagcctgaaaaccgaggacacagccgtgtattactgtaccacataccctaggtatcatgctatggactcctggggccagggcaccatggtcaccgtctcctcaggtggtggtggttctggcggcggcggctccggtggtggtggttctgccatccagttgactcagtctccatcctccctgtctgcatctgtaggagacagagtcaccatcacttgcagggccagccaaagtattagcaacaacctacactggtacctgcagaagccagggcagtctccacagctcctgatctattatggcttccagtccatctctggggtcccatcaaggttcagtggcagtggatctgggacagatttcactctcaccatcagcagtctgcaacctgaagattttgcaacttactactgtcaacaggccaacagctggccgctcacgttcggcggagggaccaagctggagatcaaagtcgaccccaaatctagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaa

SEQ ID NO: 68 - DNA, 41 bp, Artificial Sequence
  oligonucleotide primer
gtatctctcgagaaaagacaggtkcagctggtgcagtctgg

SEQ ID NO: 69 - DNA, 41 bp, Artificial Sequence
  oligonucleotide primer
gtatctctcgagaaaagacaggtccagcttgtgcagtctgg

SEQ ID NO: 70 - DNA, 41 bp, Artificial Sequence
  oligonucleotide primer
gtatctctcgagaaaagasaggtccagctggtacagtctgg

SEQ ID NO: 71 - DNA, 41 bp, Artificial Sequence
  oligonucleotide primer
gtatctctcgagaaaagacaratgcagctggtgcagtctgg

SEQ ID NO: 72 - DNA, 41 bp, Artificial Sequence
  oligonucleotide primer
gtatctctcgagaaaagacagatcaccttgaaggagtctgg

SEQ ID NO: 73 - DNA, 41 bp, Artificial Sequence
  oligonucleotide primer
gtatctctcgagaaaagacaggtcaccttgaaggagtctgg

SEQ ID NO: 74 - DNA, 39 bp, Artificial Sequence
  oligonucleotide primer
gtatctctcgagaaaagagargtgcagctggtggagtct

SEQ ID NO: 75 - DNA, 41 bp, Artificial Sequence
  oligonucleotide primer
gtatctctcgagaaaagacaggtgcagctggtggagtctgg

SEQ ID NO: 76 - DNA, 41 bp, Artificial Sequence
  oligonucleotide primer
gtatctctcgagaaaagagaggtgcagctgttggagtctgg

SEQ ID NO: 77 - DNA, 41 bp, Artificial Sequence
  oligonucleotide primer
gtatctctcgagaaaagagaggtgcagctggtgcagwcygg

SEQ ID NO: 78 - DNA, 41 bp, Artificial Sequence
  oligonucleotide primer
gtatctctcgagaaaagacagstgcagctgcaggagtcsgg

SEQ ID NO: 79 - DNA, 41 bp, Artificial Sequence
  oligonucleotide primer
gtatctctcgagaaaagacaggtgcagctacagcagtgggg

SEQ ID NO: 80 - DNA, 41 bp, Artificial Sequence
  oligonucleotide primer
gtatctctcgagaaaagagargtgcagctggtgcagtctgg

SEQ ID NO: 81 - DNA, 41 bp, Artificial Sequence
  oligonucleotide primer
gtatctctcgagaaaagacaggtacagctgcagcagtcagg

SEQ ID NO: 82 - DNA, 41 bp, Artificial Sequence
  oligonucleotide primer
gtatctctcgagaaaagacaggtgcagctggtgcaatctgg

SEQ ID NO: 83 - DNA, 48 bp, Artificial Sequence
  oligonucleotide primer
gaagacggatgggcccttggtcgacgctgaggagacrgtgaccagggt

SEQ ID NO: 84 - DNA, 48 bp, Artificial Sequence
  oligonucleotide primer
gaagacggatgggcccttggtcgacgctgaagagacggtgaccattgt

SEQ ID NO: 85 - DNA, 48 bp, Artificial Sequence
  oligonucleotide primer
gaagacggatgggcccttggtcgacgctgaggagacggtgaccgtggt

SEQ ID NO: 86 - DNA, 44 bp, Artificial Sequence
  oligonucleotide primer
gttattgctagcgttttagcaracatccagatgacccagtctcc

SEQ ID NO: 87 - DNA, 44 bp, Artificial Sequence
  oligonucleotide primer
gttattgctagcgttttagcagmcatccrgwtgacccagtctcc

SEQ ID NO: 88 - DNA, 44 bp, Artificial Sequence
  oligonucleotide primer
gttattgctagcgttttagcagtcatctggatgacccagtctcc

SEQ ID NO: 89 - DNA, 44 bp, Artificial Sequence
  oligonucleotide primer
gttattgctagcgttttagcagatattgtgatgacccagactcc

SEQ ID NO: 90 - DNA, 44 bp, Artificial Sequence
  oligonucleotide primer
gttattgctagcgttttagcagatrttgtgatgacwcagtctcc

SEQ ID NO: 91 - DNA, 44 bp, Artificial Sequence
  oligonucleotide primer
gttattgctagcgttttagcagaaattgtgttgacrcagtctcc

SEQ ID NO: 92 - DNA, 44 bp, Artificial Sequence
  oligonucleotide primer
gttattgctagcgttttagcagaaatagtgatgacgcagtctcc

SEQ ID NO: 93 - DNA, 44 bp, Artificial Sequence
  oligonucleotide primer
gttattgctagcgttttagcagaaattgtaatgacacagtctcc

SEQ ID NO: 94 - DNA, 44 bp, Artificial Sequence
  oligonucleotide primer
gttattgctagcgttttagcagacatcgtgatgacccagtctcc

SEQ ID NO: 95 - DNA, 44 bp, Artificial Sequence
  oligonucleotide primer
gttattgctagcgttttagcagaaacgacactcacgcagtctcc

SEQ ID NO: 96 - DNA, 44 bp, Artificial Sequence
  oligonucleotide primer
gttattgctagcgttttagcagaaattgtgctgactcagtctcc

SEQ ID NO: 97 - DNA, 45 bp, Artificial Sequence
  oligonucleotide primer
gcatgctcgacatcgattcactaacactctcccctgttgaagctc

SEQ ID NO: 98 - DNA, 44 bp, Artificial Sequence
  oligonucleotide primer
gttattgctagcgttttagcacagtctgtgctgactcagccacc

SEQ ID NO: 99 - DNA, 44 bp, Artificial Sequence
  oligonucleotide primer
gttattgctagcgttttagcacagtctgtgytgacgcagccgcc

SEQ ID NO: 100 - DNA, 42 bp, Artificial Sequence
  oligonucleotide primer
gttattgctagcgttttagcacagtctgccctgactcagcct

SEQ ID NO: 101 - DNA, 42 bp, Artificial Sequence
  oligonucleotide primer
gttattgctagcgttttagcatcctatgwgctgacwcagcca

SEQ ID NO: 102 - DNA, 44 bp, Artificial Sequence
  oligonucleotide primer
gttattgctagcgttttagcatcctatgagctgacacagctacc

SEQ ID NO: 103 - DNA, 43 bp, Artificial Sequence
  oligonucleotide primer
gttattgctagcgttttagcatcttctgagctgactcaggacc

SEQ ID NO: 104 - DNA, 43 bp, Artificial Sequence
  oligonucleotide primer
gttattgctagcgttttagcatcctatgagctgatgcagccac

SEQ ID NO: 105 - DNA, 41 bp, Artificial Sequence
  oligonucleotide primer
gttattgctagcgttttagcacagcytgtgctgactcaatc

SEQ ID NO: 106 - DNA, 41 bp, Artificial Sequence
  oligonucleotide primer
gttattgctagcgttttagcacwgsctgtgctgactcagcc

SEQ ID NO: 107 - DNA, 44 bp, Artificial Sequence
  oligonucleotide primer
gttattgctagcgttttagcaaattttatgctgactcagcccca

SEQ ID NO: 108 - DNA, 44 bp, Artificial Sequence
  oligonucleotide primer
gttattgctagcgttttagcacagrctgtggtgacycaggagcc

SEQ ID NO: 109 - DNA, 44 bp, Artificial Sequence
  oligonucleotide primer
gttattgctagcgttttagcacaggcagggctgactcagccacc

SEQ ID NO: 110 - DNA, 46 bp, Artificial Sequence
  oligonucleotide primer
gcatgctcgacatcgattcactatgaacattctgtaggggccactg

SEQ ID NO: 111 - DNA, 46 bp, Artificial Sequence
  oligonucleotide primer
gcatgctcgacatcgattcactaagagcattctgcaggggccactg

SEQ ID NO: 112 - DNA, 31 bp, Artificial Sequence
  oligonucleotide primer
aaaagaatgagatttccttcaatttttactg

SEQ ID NO: 113 - DNA, 19 bp, Artificial Sequence
  oligonucleotide primer
gcgtgaatgtaagcgtgac

SEQ ID NO: 114 - DNA, 24 bp, Artificial Sequence
  oligonucleotide primer
acaacagaagatgaaacggcacaa

SEQ ID NO: 115 - DNA, 21 bp, Artificial Sequence
  oligonucleotide primer
atggtgatggtgatggtgtct

SEQ ID NO: 116 - DNA, 21 bp, Artificial Sequence
  oligonucleotide primer
agacaccatcaccatcaccat

SEQ ID NO: 117 - DNA, 19 bp, Artificial Sequence
  oligonucleotide primer
taatgcagggccgcagctt

SEQ ID NO: 118 - DNA, 21 bp, Artificial Sequence
  oligonucleotide primer
cggcacaaattccggctgaag

SEQ ID NO: 119 - DNA, 19 bp, Artificial Sequence
  oligonucleotide primer
tcactgcttcttgcgcagg

SEQ ID NO: 120 - DNA, 32 bp, Artificial Sequence
  oligonucleotide primer
ggcccaaaactgacactttaaacgctgtcttg

SEQ ID NO: 121 - DNA, 32 bp, Artificial Sequence
  oligonucleotide primer
caagacagcgtttaaagtgtcagttttgggcc

SEQ ID NO: 122 - DNA, 64 bp, Artificial sequence
  oligonucleotide primer
cggcagcaacatgttcgagataagctgggggaacattcgcgaaaatgaaacaagtcggctgtta

SEQ ID NO: 123 - DNA, 62 bp, artificial sequence
  oligonucleotide primer
atgaaacaagtcggctgttatagtatatttattataatattgaaagatctcaaaagactact

SEQ ID NO: 124 - DNA, 68 bp, Artificial sequence
  oligonucleotide primer
cgtgctgcacatgttcgacaattaatatttacttattttggtcaaccccaaataggttgatttcatac

SEQ ID NO: 125 - DNA, 58 bp, Artificial sequence
  oligonulceotide primer
caaataggttgatttcatacttggttcattcaaaaataagtagtcttttgagatcttt

(19) United States
(12) Patent Application Publication
(10) Pub. No.: US 2011/0076752 A1
(43) Pub. Date: Mar. 31, 2011

(54) ANTIBODY LIBRARY DISPLAY BY YEAST CELL PLASMA MEMBRANE

(75) Inventors: Herren Wu, Boyds, MD (US); Changshou Gao, Potomac, MD (US)

(73) Assignee: MEDIMMUNE, LLC, Gaithersburg, MD (US)

(21) Appl. No.: 12/526,391

(22) PCT Filed: Feb. 8, 2008
(86) PCT No.: PCT/US08/53398

§ 371 Date: Dec. 9, 2010

Related U.S. Application Data
(60) Provisional application No. 60/889,019, filed on Feb. 9, 2007.

(51) Int. Cl. C07K 1/00; C40B 40/06; C40B 40/10; C40B 40/02; C40B 50/06; C07K 16/00

(52) U.S. Cl. 435/272; 506/16; 506/18; 506/14; 506/26; 530/387.1

(57) ABSTRACT

The present invention relates to antibodies or antibody fragments that may be displayed on the extracellular surface of the plasma membrane when expressed in a host cell. The present invention provides libraries comprising a plurality of plasma membrane displayed antibodies and methods of screening the libraries for antibodies or antibody fragments with desired characteristics.

### 1. BACKGROUND OF THE INVENTION

Recombinant antibodies have become increasingly prevalent therapeutics over the past decade; currently they represent over 30% of biopharmaceuticals in clinical trials. As such, the rapid generation, characterization and optimization of recombinant antibodies is critically important for the biopharmaceutical industry. An early solution for the problem was provided by the use of phage display libraries of simplified antibody fragments. The possibility to generate large libraries and the ease with which a library is screened made antibody fragment phage display technology a powerful tool for the development of new therapeutics against various human diseases.

Phage display technologies, however, have a disadvantage in that they rely on the screening of antibody fragments as opposed to full length antibodies. Given that most therapeutic applications call for the use of divalent IgG antibodies, an isolated antibody fragment with the desired binding properties is usually converted into a full length antibody. The conversion process is not only labor intensive, but may also result in the loss of antigen binding specificity.

Additional display technologies have been developed to address these problems. Recently, the a type agglutinin mediated display of antibody fragments (scFv and Fab) on the yeast cell wall has been emerging as an effective alternative to the phage display technology.

There is still a need, however, in the art for the development of new technologies that allow the rapid screening of large libraries of full length antibodies.

### 2. SUMMARY OF THE INVENTION

The present invention relates to an antibody or a fragment thereof that may be displayed on the extracellular surface of the plasma membrane of a cell (e.g., yeast cell), referred to herein as an “antibody of the invention” and like terms. In certain embodiments, an antibody of the invention comprises a heavy chain or a fragment thereof and optionally a light chain or a fragment thereof, wherein either the heavy chain or light chain further comprises an amino acid sequence that targets the antibody or a fragment thereof to the extracellular surface of the plasma membrane. In one embodiment, an antibody of the invention comprises a full length heavy chain having an amino acid sequence that targets the antibody to the extracellular surface of the plasma membrane, wherein said amino acid sequence is fused to the C terminus of said heavy chain, and wherein said antibody may further comprise a full length light chain. In still another embodiment, an antibody of the invention comprises a portion of a heavy chain having an amino acid sequence that targets the antibody to the extracellular surface of the plasma membrane, wherein said amino acid sequence is fused to the C terminus of said heavy chain portion, and wherein said antibody may further comprise a light chain or a fragment thereof. In a specific embodiment, said amino acid sequence that targets an antibody of the invention to the extracellular surface of the plasma membrane is a transmembrane domain. In another embodiment, said amino acid sequence that targets an antibody of the invention to the extracellular surface of the plasma membrane is a GPI anchor domain.

The present invention further relates to vectors comprising polynucleotides encoding an antibody or a fragment thereof that may be displayed on the extracellular surface of the plasma membrane of a cell (e.g., yeast cell), referred to herein as a “vector of the invention”. In one embodiment, a vector of the invention is operable in a host cell to direct the expression and the display of an antibody or a fragment thereof on the extracellular surface of the plasma membrane. In a specific embodiment, a vector of the invention is a set of two vectors wherein a first vector comprises a polynucleotide encoding a heavy chain of an antibody or a fragment thereof and a second vector comprises a polynucleotide encoding a light chain of an antibody or a fragment thereof, wherein said antibody or a fragment thereof may be displayed on the extracellular surface of the plasma membrane.

The present invention also provides host cells comprising an antibody or a fragment thereof that may be displayed on the extracellular surface of the plasma membrane, referred to herein as a “host cell of the invention”. In one embodiment, a host cell of the invention is a eukaryotic cell selected from the Ascomycota phylum. In a specific embodiment, a host cell of the invention is Saccharomyces cerevisiae, Hansenula polymorpha, Kluyveromyces lactis, Pichia pastoris, Schizosaccharomyces pombe, or Yarrowia lipolytica. In one embodiment, a host cell of the invention comprises a genetic mutation wherein said genetic mutation renders the cell wall permeable to an antibody binding agent (e.g. antigens, Fc receptors, antibodies).

The present invention also relates to libraries comprising polynucleotides encoding a heterogeneous population of antibodies or a fragment thereof that may be displayed on the extracellular surface of the plasma membrane of a cell (e.g., yeast cell), referred to herein as a “polynucleotide library of the invention”. In one embodiment, a polynucleotide library of the invention may comprise polynucleotides encoding antibodies or a fragment thereof comprising a heterogeneous population of heavy chain variable regions. In another embodiment, a polynucleotide library of the invention may comprise polynucleotides encoding antibodies or a fragment thereof comprising a heterogeneous population of light chain variable regions. In a further embodiment, a polynucleotide library of the invention may comprise polynucleotides encoding antibodies or a fragment thereof comprising a heterogeneous population of Fc regions, including variant Fc regions. In one embodiment, a population of host cells comprises a polynucleotide library of the invention.

The present invention also relates to libraries comprising a heterogeneous population of antibodies or a fragment thereof that may be displayed on the extracellular surface of the plasma membrane of a cell (e.g., yeast cell), referred to herein as an “antibody library of the invention”. In one embodiment, an antibody library of the invention may comprise a heterogeneous population of heavy chain variable regions. In another embodiment, an antibody library of the invention may comprise a heterogeneous population of light chain variable regions. In a further embodiment, an antibody library of the invention may comprise a heterogeneous population of Fc regions, including variant Fc regions. In one embodiment, a population of host cells comprises an antibody library of the invention.

The invention also provides methods of screening a polynucleotide library of the invention or an antibody library of the invention. In one embodiment, a method of screening a library allows the identification of an antibody or a fragment thereof that binds a specific antigen. In one embodiment, a method of screening a library allows the identification of an antibody or a fragment thereof having an altered binding to a specific antigen. In one embodiment, a method of screening a library allows the identification of an antibody or fragment having an altered binding to effector molecules (e.g., FcγRs and/or C1q).

The invention also provides methods of expressing a polynucleotide library of the invention in a host cell. In one embodiment, a polynucleotide library of the invention may encode a heterogeneous population of antibodies or a fragment thereof that may be displayed on the extracellular surface of the plasma membrane of a cell (e.g., yeast cell).

### 3. BRIEF DESCRIPTION OF THE FIGURES

For the purpose of illustrating representative embodiments of the invention, drawings are provided herein.

FIG. 1. Schematic representation of heavy chain fusion polypeptides of an antibody of the invention. Panel A) depicts a heavy chain targeted for display on the extracellular surface of the yeast plasma membrane. It comprises a signal sequence (SS), a heavy chain variable region (VH), a heavy chain constant region 1 (CH1), a hinge region (H), a heavy chain constant region 2 (CH2), a heavy chain constant region 3 (CH3), and a transmembrane domain (TM). The amino acid sequence of the human thrombomodulin transmembrane domain is shown as a non limiting example (SEQ ID NO:2). Panel B) depicts a heavy chain targeted for display on the yeast cell wall. It comprises a signal sequence (SS), a heavy chain variable region (VH), a heavy chain constant region 1 (CH1), a hinge region (H), a heavy chain constant region 2 (CH2), a heavy chain constant region 3 (CH3), and a GPI anchor domain (GPI). The GPI anchor domain comprising residues 320-650 of the Aga1 protein is given as a non limiting example.

FIG. 2. (A) Schematic representation of a yeast vector that may be used to control the expression of the heavy chain of an antibody or a fragment thereof that may be displayed on the surface of a yeast cell. (B) Schematic representation of a yeast vector that may be used to control the expression of the light chain of an antibody or a fragment thereof that may be displayed on the surface of a yeast cell.

FIG. 3. Fluorescence intensity profile of immunostained spheroplasts expressing the 10C12 light chain and a cell surface displayed heavy chain fusion polypeptide. “Aga1p GPI”, “hThrm TM”, “Axl2p TM”, and “Swp1p TM” denotes the data obtained with spheroplasts expressing the 10C12 heavy chain fused with the Aga1p GPI signal, the human thrombomodulin transmembrane region, the Axl2p transmembrane region, and the Swp1p transmembrane region, respectively. Spheroplasts not expressing an antibody serve as negative control (“Uninduced”). Samples were stained with FITC conjugated anti-human IgG(H+L) antibody. “hThrm TM”, “Axl2p TM”, and “Swp1p TM” samples show significant staining; the staining profile of “Aga1p GPI” resembles that of the negative control sample.

FIG. 4. Mean fluorescence intensity of immunostained spheroplasts. Spheroplasts expressing the 10C12 light chain and a 10C12 heavy chain fused to either human thrombomodulin transmembrane domain (10C12-hThrMTM, light gray bars) or Aga1 GPI signal (10C12-AgaGPI, dark grey bars) were immunostained with FITC conjugated anti-human IgG(H+L) antibody. Spheroplasts were generated by lyticase treatment at the indicated pH in the presence or absence of 0.5 M sorbitol. Control samples of immunostained intact yeast cells (“no lyticase”) were also analyzed. The hThrmTM fused 10C12 antibody is positively stained on spheroplasts, but not intact yeast cells. Lyticase treatment, on the other hand, significantly reduces the signal obtained with the AgaGPI fused antibody.

FIG. 5. Fluorescence intensity profile of immunostained intact yeast cells expressing the 10C12 light chain and the 10C12 heavy chain fused to the Aga1 GPI signal (10C12-AgaGPI); and immunostained spheroplasts expressing the 10C12 light chain and the 10C12 heavy chain fused to the human thrombomodulin transmembrane domain (10C12-hThrMTM). Samples were stained with biotinylated EphA4-Fc, a ligand for 10C12, and PE conjugated streptavidin. Spheroplasts without 10C12 expression were included as a negative control (“uninduced”). Positive staining is detected for both the GPI anchored and ThrmTM anchored 10C12 antibody.

FIG. 6. Fluorescence intensity profile of immunostained spheroplasts expressing the 3F2 anti-EphA2 Fab fused to the human thrombomodulin transmembrane domain (hThrmTM). hThrmTM is fused to either the heavy chain (3F2Fab-hThrmTM(HC)) or light chain (3F2Fab-hThrmTM(LC)) of the Fab. Spheroplasts without Fab expression were included as a negative control (“uninduced”). Spheroplasts were stained with (A) biotinylated EphA2-Fc/PE conjugated streptavidin or (B) FITC conjugated anti-IgG(H+L). The heavy chain and light chain anchored Fabs are stained equally well with FITC conjugated anti-IgG(H+L); they also bind the biotinylated EphA2-Fc ligand.

FIG. 7. Fluorescence intensity profile of immunostained spheroplasts expressing the 3F2 scFv comprising a FLAG tag and a human thrombomodulin transmembrane domain fused to its N and C terminus, respectively. Spheroplasts were stained with a FITC conjugated anti-FLAG antibody; spheroplasts without scFv expression were used as negative control (“uninduced”). 3F2 scFv is readily detected on the cell surface.

FIG. 8. Fluorescence intensity profile of immunostained intact yeast cells. MAT alpha, mnn9, ura3, leu2, his4 yeast cells expressing the 3F2 anti-EphA2 scFv-Fc fused to the human thrombomodulin transmembrane domain (3F2scFv-Fc-hThrmTM) or the 3F2 heavy chain fused to the human thrombomodulin transmembrane domain (3F2HC-hThrmTM) were stained with FITC conjugated anti-human Fc antibody. Cells without an expression construct were used as negative controls. Cells expressing 3F2scFv-Fc-hThrmTM, but not 3F2HC-hThrmTM show positive staining.

FIG. 9. Fluorescence intensity profile of immunostained intact yeast cells. MAT alpha, mnn9, ura3, leu2, his4 yeast cells expressing the 3F2 anti-EphA2 scFv-Fc fused to the human thrombomodulin transmembrane domain (3F2scFv-Fc-hThrmTM) or the 3F2 heavy chain fused to the human thrombomodulin transmembrane domain (3F2HC-hThrmTM) were stained with biotinylated EphA2/PE conjugated streptavidin. Cells without an expression construct were used as negative controls. Cells expressing 3F2scFv-Fc-hThrmTM, but not 3F2HC-hThrmTM show positive staining.

FIG. 10. Schematic representation of the 2μ-scFv-TM vector.

FIG. 11. Experimental flow chart of a single round of antibody selection.

FIG. 12. Efficiency of antibody gene amplification from plasmid DNA purified from yeast spheroplast. PCR product amplified form DNA purified from 1000, 2000, 3000, 4000, 5000, 6000, and 10000 cells were run in lanes 1 to 7, respectively, on an agarose gel. Lane 8 shows the PCR product of direct amplification from 2000 cells. All PCR reactions yielded a DNA fragment of the same size. The staining intensity of the various PCR products was comparable.

FIG. 13. Fluorescent intensity profile of various artificial libraries of antibody displaying spheroplasts during the first round of selection.

FIG. 14. Fluorescent intensity profile of various artificial libraries of antibody displaying spheroplasts during the second round of selection.

FIG. 15. Schematic representation of the pPICZ+scFv-FC vector.

FIG. 16. Fluorescent intensity profile of EphA4-Fc-biotin/streptavidin-PE stained P. pastoris expressing a GPI anchored 10C12 anti-EphA4 scFv, scFv-Fc or full IgG on the cell surface. The mean fluorescence intensity of the antibody expressing yeast cells is separated from the parental negative control yeast cells by at least 2 logs.

FIG. 17. Zymolase digestion dependence of antigen binding by membrane anchored 10C12 anti-EphA4 His/FLAG-tagged-scFv (10C12ScFvHF-TM) or scFv-Fc (10C12ScFvFc-TM) molecules expressed in P. pastoris. Spheroplasts prepared by 0 min, 2 min, 5 min, 10 min, 20 min or 30 min zymolase digestion (2.5 units zymolase per 10⁸ cells) were stained with EphA4-Fc-biotin/streptavidin-PE. Fluorescent intensity profile of the stained spheroplasts is shown. Similarly stained parental P. pastoris spheroplasts were included as negative control. The fluorescent intensity profile of TM anchored scFv and scFv-Fc expressing cells is almost identical at all time points tested. The separation between the mean fluorescent intensity (MFI) of antibody expressing and control cells reaches a 2 log maximum after 2 minutes of zymolase digestion.

FIG. 18. P. pastoris expressed TM anchored 10C12 anti-EphA4 scFv or scFv-Fc molecules are accessible to antigen binding in absence of zymolase treatment. Cells were washed with PBS or 50 mM DTT/1 M sorbitol, incubated with 10 μg/ml, 1 μg/ml or 0.1 μg/ml EphA4-Fc-biotin, and stained with streptavidin-PE. Similarly treated parental P. pastoris cells were included as negative control. The fluorescent intensity profile of PBS washed TM anchored scFv and scFv-Fc expressing cells are identical at each concentration of EphA4-Fc-biotin tested; the separation between the MFI of antibody expressing cells and parental cells is not dependent on antigen concentration within the range tested. The fluorescent intensity profile of DTT/sorbitol washed TM anchored scFv-Fc expressing cells is higher than that of the scFv expressing cells at all EphA4-Fc-biotin concentrations tested; the separation between the MFI of antibody expressing cells and parental cells is not dependent on antigen concentration within the range tested.

FIG. 19. Episomal expression of P. pastoris surface displayed antibodies. Fluorescent intensity profile of 10 ug/ml EphA4-Fc-biotin/streptavidin-PE stained cells. Surface displayed antibodies were expressed from an episomal vector. Similarly treated parental P. pastoris cells were included as negative control.

### 4. DEFINITIONS

As used herein, the term “polynucleotide encoding an antibody or a fragment thereof” encompasses a composition of polynucleotides comprising one or more polynucleotide chains encoding the individual polypeptide chains of said antibody or a fragment thereof.

As used herein, “vector” refers to any element capable of serving as a vehicle of genetic transfer, gene expression, or replication or integration of a foreign polynucleotide in a host cell. A vector can be an artificial chromosome or plasmid, and can be integrated into the host cell genome or exist as an independent genetic element (e.g., episome, plasmid). A vector can exist as a single polynucleotide or as two or more separate polynucleotides. A “vector of the invention” is capable, in an appropriate host, of directing expression of at least one chain of an antibody or fragment thereof that may be displayed on the plasma membrane of the host cell. Vectors according to the present invention can be single copy vectors or multicopy vectors (indicating the number of copies of the vector typically maintained in the host cell). Vectors of the present invention include yeast expression vectors, 2u vectors and centromere vectors. A “shuttle vector” (or bi-functional vector) is known in the art as any vector that can replicate in more than one species of organism. For example, a shuttle vector that can replicate in both Escherichia coli (E. coli) and Saccharomyces cerevisiae (S. cerevisiae) can be constructed by linking sequences from an E. coli plasmid with sequences from the yeast 2μ plasmid.

The term “library” refers to a mixture of heterogeneous polypeptides or polynucleotides. A library is composed of members that have similar polypeptide or polynucleotide sequences. Where the library is a polynucleotide library, it encodes a heterogeneous population of polypeptides (e.g., a heterogeneous population of antibody polypeptides). Sequence differences between library members are responsible for the diversity present in the library. The library can take the form of a simple mixture of polypeptides or polynucleotides, or can be in the form of organisms or cells, for example yeast cells and the like, that are transformed with a library of polynucleotides. Advantageously, polynucleotides are incorporated into expression vectors, in order to allow expression of the polypeptides encoded by the polynucleotides. In one embodiment, a library can take the form of a population of host cells, each cell containing one or more copies of an expression vector containing a one or more polynucleotide encoding an antibody or a fragment thereof that can be expressed to produce its corresponding antibody or a fragment thereof. Thus, the population of host cells has the potential to encode a heterogeneous population of antibodies or a fragment thereof.

As used herein, the terms “antibody” and “antibodies” refer to monoclonal antibodies, multispecific antibodies, human antibodies, humanized antibodies, camelised antibodies, chimeric antibodies, single-chain Fvs (scFv), disulfide-linked Fvs (sdFv), Fab fragments, F (ab′) fragments, and anti-idiotypic (anti-Id) antibodies (including, e.g., anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site, these fragments may or may not be fused to another immunoglobulin domain including but not limited to, an Fc region or a fragment thereof. The term “antibody” and “antibodies” also encompass any polypeptide comprising an immunoglobulin molecule or an immunologically active fragment thereof (i.e., molecules that contain an antigen binding site) fused to any polypeptide fusion partner. As used herein, the terms “antibody” and “antibodies” also include the Fc variants, full-length antibodies and Fc variant-fusions comprising Fc regions, or a fragment thereof. Fc variant-fusions include but are not limited to, scFv-Fc fusions, variable region (e.g., VL and VH) -Fc fusions, scFv-scFv-Fc fusions. Antibodies can be of any type (e.g., IgG, IgE, IgM, IgD, IgA and IgY), class (e.g., IgG1, IgG2, IgG3, IgG4, IgA1 and IgA2) or subclass.

The complementarity determining regions (CDRs) residue numbers referred to herein are those of Kabat et al. (1991, NIH Publication 91-3242, National Technical Information Service, Springfield, Va.). Specifically, residues 24-34 (CDR1), 50-56 (CDR2) and 89-97 (CDR3) in the light chain variable domain and 31-35 (CDR1), 50-65 (CDR2) and 95-102 (CDR3) in the heavy chain variable domain. Note that CDRs vary considerably from antibody to antibody (and by definition will not exhibit homology with the Kabat consensus sequences). Maximal alignment of framework residues frequently requires the insertion of “spacer” residues in the numbering system, to be used for the Fv region. It will be understood that the CDRs referred to herein are those of Kabat et al. supra. In addition, the identity of certain individual residues at any given Kabat site number may vary from antibody chain to antibody chain due to interspecies or allelic divergence.

As used herein “Fc region” includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region. Also encompassed are proteins comprising variant Fc regions, which are non-naturally occurring variants of an Fc region. The amino acid sequence of a non-naturally occurring Fc region (also referred to herein as a “variant Fc region”) comprises a substitution, insertion and/or deletion of at least one amino acid residue compared to the wild type amino acid sequence. Any new amino acid residue appearing in the sequence of a variant Fc region as a result of an insertion or substitution may be referred to as a non-naturally occurring amino acid residue. Note: Polymorphisms have been observed at a number of Fc positions, including but not limited to Kabat 270, 272, 312, 315, 356, and 358, and thus slight differences between the presented sequence and sequences in the art may exist.

As used herein, the term “transmembrane domain” refers to the domain of a peptide, polypeptide or protein that is capable of spanning the plasma membrane of a cell. These domains can be used to anchor an antibody on the plasma membrane.

A “chimeric antibody” is a molecule in which different portions of the antibody are derived from different immunoglobulin molecules such as antibodies having a variable region derived from a non-human antibody and a human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, and 4,816,397, CDR-grafting (EP 239,400; PCT Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5): 489-498; Studnicka et al., 1994, Protein Engineering 7:805; and Roguska et al., 1994, PNAS 91:969), and chain shuffling (U.S. Pat. No. 5,565,332).

A “humanized antibody” is an antibody or its variant or a fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′)2, Fabc, Fv) in which all or substantially all of the CDR regions correspond to those of a non-human immunoglobulin (i.e., donor antibody) and all or substantially all of the framework regions are those of a human immunoglobulin consensus sequence. In one embodiment, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including, but not limited to, IgM, IgG, IgD, IgA and IgE, and any isotype, including, but not limited to, IgG1, IgG2, IgG3 and IgG4. In another embodiment, the constant domain is a complement fixing constant domain where it is desired that the humanized antibody exhibit cytotoxic activity, and the class is typically IgG1. Where such cytotoxic activity is not desirable, the constant domain may be of the IgG 2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. In one embodiment, at least 75%, at least 90%, and or at least 95% of the humanized antibody residues will correspond to those of the parental framework region (FR) and CDR sequences. Humanized antibody can be produced using variety of techniques known in the art, including but not limited to, CDR-grafting (European Patent No. EP 239,400; PCT Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5): 489-498; Studnicka et al., 1994, Protein Engineering 7(6): 805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, PCT Patent Publication WO 93/17105, Tan et al., 2002, J. Immunol. 169:1119-25, Caldas et al., 2000, Protein Eng. 13: 353-60, Morea et al., 2000, Methods 20: 267-79, Baca et al., 1997, J. Biol. Chem. 272: 10678-84, Roguska et al., 1996, Protein Eng. 9: 895-904, Couto et al., 1995, Cancer Res. 55 (23 Supp): 5973s -5977s, Couto et al., 1995, Cancer Res. 55: 1717-22, Sandhu J S, 1994, Gene 150: 409-10, and Pedersen et al., 1994, J. Mol. Biol. 235: 959-73). Often, framework residues in the framework regions will be substituted with the corresponding residue from the CDR donor antibody to alter and/or improve antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323).

The term “ADCC” (antibody-dependent cell-mediated cytotoxicity) refers to a cell-mediated reaction in which nonspecific cytotoxic cells that express Fc receptors (FcR) (e.g. natural killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. To assess ADCC activity of a molecule of interest, an in vitro ADCC assay (e.g. such as that described in U.S. Pat. No. 5,500,362 and U.S. Pat. No. 5,821,337) may be performed. Useful effector cells for such assays include peripheral blood mononuclear cells (PBMC) and natural killer (NK) cells.

“Complement dependent cytotoxicity” and “CDC” refer to the lysing of a target cell in the presence of complement. The complement activation pathway is initiated by the binding of the first component of the complement system (Clq) to a molecule, an antibody for example, complexed with a cognate antigen. To assess complement activation, a CDC assay, e.g. as described in Gazzano-Santoro et al., 1996, J. Immunol. Methods, 202:163, may be performed.

### 5. DETAILED DESCRIPTION

The present invention relates to an antibody or a fragment thereof that may be displayed on the extracellular surface of the plasma membrane of a cell (e.g., yeast cell), referred to herein as an “antibody of the invention” and like terms.

In one embodiment, an antibody of the invention is a murine antibody, a primate antibody, a chimeric antibody, a primatized antibody, a humanized antibody or a human antibody. In one embodiment, an antibody of the invention is a human antibody.

In one embodiment, an antibody of the invention is of an immunoglobulin type selected from the group consisting of IgA, IgE, IgM, IgD, IgY and IgG.

In one embodiment, an antibody of the invention comprises a heavy chain or a fragment thereof having an amino acid sequence that targets the antibody to the extracellular surface of the plasma membrane of a cell (e.g., yeast cell). In one embodiment, an antibody of the invention comprises a heavy chain or a fragment thereof having an amino acid sequence that targets the antibody to the extracellular surface of the plasma membrane, wherein said amino acid sequence is fused to the C terminal end of said heavy chain or a fragment thereof.

In another embodiment, an antibody of the invention comprises a light chain or a fragment thereof having an amino acid sequence that targets the antibody to the extracellular surface of the plasma membrane of a cell (e.g., yeast cell). In a specific embodiment, an antibody of the invention comprises a light chain or a fragment thereof having an amino acid sequence that targets the antibody to the extracellular surface of the plasma membrane, wherein said amino acid sequence is fused to the C terminal end of said light chain or a fragment thereof.

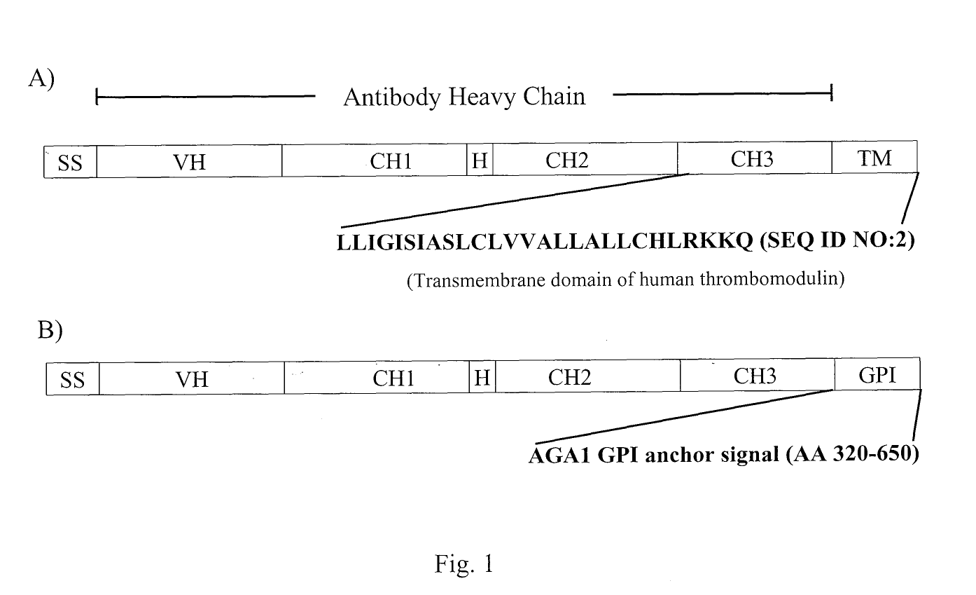

In yet another embodiment, an antibody of the invention comprises a single chain antigen binding domain, including but not limited to an scFv, having an amino acid sequence that targets the single chain antibody to the extracellular surface of the plasma membrane of a cell (e.g., yeast cell). In certain embodiments, the amino acid sequence that targets the single chain antibody to the extracellular surface of the plasma membrane of a cell is a transmembrane domain or a GPI anchor domain (FIG. 1). In certain embodiments, the single chain antibody is fused to an Fc region and said membrane targeting domain is fused to the C-terminus of the single chain antibody Fc fusion protein.

In one embodiment, said amino acid sequence that targets the antibody to the extracellular surface of the plasma membrane is a transmembrane domain (FIG. 1). In another embodiment, said amino acid sequence that targets the antibody to the extracellular surface of the plasma membrane is a GPI anchor domain.

In one embodiment, an antibody of the invention comprises a heavy chain or a fragment thereof having a transmembrane domain that targets the antibody to the extracellular surface of the plasma membrane of a cell (e.g., yeast cell). In a specific embodiment, an antibody of the invention comprises a heavy chain or a fragment thereof having a transmembrane domain that targets the antibody to the extracellular surface of the plasma membrane, wherein said transmembrane domain is fused to the C terminal end of said heavy chain or a fragment thereof.

In one embodiment, a cell surface displayed antibody or a fragment thereof of the current invention comprises the transmembrane domain of thrombomodulin having an amino acid sequence of SEQ ID NO:2 or a functional fragment thereof. In another embodiment, a cell surface displayed antibody or a fragment thereof of the current invention comprises a transmembrane domain that is at least 70%, or at least 80%, or at least 90%, or at least 95%, or at least 97%, or at least 99% identical to SEQ ID NO:2.

In one embodiment, a cell surface displayed antibody or a fragment thereof of the current invention comprises the transmembrane domain of Axl2p having an amino acid sequence of SEQ ID NO:4 or a functional fragment thereof. In another embodiment, a cell surface displayed antibody or a fragment thereof of the current invention comprises a transmembrane domain that is at least 70%, or at least 80%, or at least 90%, or at least 95%, or at least 97%, or at least 99% identical to SEQ ID NO:4.

In one embodiment, a cell surface displayed antibody or a fragment thereof of the current invention comprises the transmembrane domain of Swp1p having an amino acid sequence of SEQ ID NO:6 or a functional fragment thereof. In another embodiment, a cell surface displayed antibody or a fragment thereof of the current invention comprises a transmembrane domain that is at least 70%, or at least 80%, or at least 90%, or at least 95%, or at least 97%, or at least 99% identical to SEQ ID NO:6.

In another embodiment, an antibody of the invention comprises a light chain or a fragment thereof having a transmembrane domain that targets the antibody to the extracellular surface of the plasma membrane of a cell (e.g., yeast cell). In a specific embodiment, an antibody of the invention comprises a light chain or a fragment thereof having a transmembrane domain that targets the antibody to the extracellular surface of the plasma membrane, wherein said transmembrane domain is fused to the C terminal end of said light chain or a fragment thereof.

In one embodiment, an antibody of the invention comprises a full length heavy chain having an amino acid sequence that targets the antibody to the extracellular surface of the plasma membrane of a cell (e.g., yeast cell), wherein said amino acid sequence is fused to the C terminus of said heavy chain; and may further comprise a full length light chain or a fragment thereof. In still another embodiment, an antibody of the invention comprises a portion of a heavy chain having an amino acid sequence that targets the antibody to the extracellular surface of the plasma membrane, wherein said amino acid sequence is fused to the C terminus of said heavy chain portion; and may further comprise a light chain or a fragment thereof. In yet another embodiment, an antibody of the invention comprises a single chain antigen binding domain, including but not limited to an scFv, having an amino acid sequence that targets the antibody to the extracellular surface of the plasma membrane of a cell (e.g., yeast cell), wherein said amino acid sequence is fused to either the N-terminus or C-terminus of the single chain antibody. In certain embodiments, the single chain antibody is fused to an Fc region and said amino acid sequence is fused to the either the N-terminus or C-terminus of the single chain antibody Fc fusion protein.

In one embodiment, an antibody of the invention comprises a full length heavy chain having a transmembrane domain that targets the antibody to the extracellular surface of the plasma membrane of a cell (e.g., yeast cell), wherein said transmembrane domain is fused to the C terminus of said heavy chain; and may further comprise a full length light chain or a fragment thereof. In still another embodiment, an antibody of the invention comprises a portion of a heavy chain having a transmembrane domain that targets the antibody to the extracellular surface of the plasma membrane, wherein said transmembrane domain is fused to the C terminus of said heavy chain portion; and may further comprise a light chain or a fragment thereof. In yet another embodiment, an antibody of the invention comprises a single chain antigen binding domain, including but not limited to an scFv, having a transmembrane domain that targets the single chain antibody to the extracellular surface of the plasma membrane of a cell (e.g., yeast cell), wherein said transmembrane domain is fused to either the N-terminus or C-terminus of the single chain antibody. In certain embodiments, the single chain antibody is fused to an Fc region and said transmembrane domain is fused to the either the N-terminus or C-terminus of the single chain antibody Fc fusion protein.

The current invention also relates to polynucleotides encoding an antibody or a fragment thereof that may be displayed on the extracellular surface of the plasma membrane of a cell (e.g., yeast cell), referred to herein as a “polynucleotide of the invention”. In one embodiment, a polynucleotide of the invention comprises two operatively linked coding regions, wherein a first coding region encodes an antibody polypeptide or a fragment thereof and a second coding region encodes a transmembrane domain. In another embodiment, a polynucleotide of the invention comprises two operatively linked coding regions, wherein said coding regions are immediately juxtaposed. In a further embodiment, a polynucleotide of the invention comprises two operatively linked coding regions, wherein said coding regions are separated by at least one codon.

The present invention further relates to vectors comprising a polynucleotide encoding an antibody or a fragment thereof that may be displayed on the extracellular surface of the plasma membrane of a cell (e.g., yeast cell), referred to herein as a “vector of the invention”. In one embodiment, a vector of the invention is a shuttle vector. In a particular embodiment, a vector of the invention is a shuttle vector that is capable of replication in an E. coli host cell, as well as in a eukaryotic host cell capable of expressing an antibody of the invention. In a further embodiment, a vector of the invention is a shuttle vector that is capable of replication in an E. coli host cell and in a yeast host cell. In a specific embodiment, a vector of the invention is a shuttle vector that is capable of replication in an E. coli host cell and in a S. cerevisiae host cell.

In one embodiment, a vector of the invention is an episomal vector. In another embodiment, a vector of the invention is a integrating vector. In one embodiment, a vector of the invention is a single copy vector. In another embodiment, a vector of the invention is a low copy number vector. In a further embodiment, a vector of the invention is a high copy number vector. In a specific embodiment, a vector of the invention is an autonomously replicating low copy-number yeast vector.

In one embodiment, a vector of the invention is operable in a host cell to direct the expression and display of an antibody or a fragment thereof on the extracellular surface of the plasma membrane of a cell (e.g., yeast cell). In one embodiment, a vector of the invention may comprise a promoter or a transcription terminator operatively linked to a polynucleotide encoding a plasma membrane displayed antibody or a fragment thereof. In another embodiment, a vector of the invention may comprise a first and second polynucleotide wherein said first polynucleotide encoding a signal sequence peptide is operatively linked to a second polynucleotide encoding an antibody or a fragment thereof that may be displayed on the plasma membrane.

In one embodiment, a vector of the invention is a single vector. In another embodiment, a vector of the invention is a set of vectors. In a further embodiment, a vector of the invention is a set of two vectors wherein a first vector comprises a polynucleotide encoding a heavy chain of an antibody or a fragment thereof and a second vector comprises a polynucleotide encoding a light chain of an antibody or a fragment thereof, wherein said antibody or a fragment thereof may be displayed on the extracellular surface of the plasma membrane.

The present invention also relates to host cells comprising an antibody or a fragment thereof displayed on the extracellular surface of the plasma membrane, referred to herein as a “host cell of the invention”. In one embodiment, a host cell of the invention is a eukaryotic cell selected from the Ascomycota phylum. In another embodiment, a host cell of the invention is a cell belonging to the Saccharomyces, Pichia, Hansenula, Schizosaccharomyces, Kluyveromyces, Yarrowia, or Candida genera. In a specific embodiment, a host cell of the invention is Saccharomyces cerevisiae, Hansenula polymorpha, Kluyveromyces lactis, Pichia pastoris, Schizosaccharomyces pombe, or Yarrowia lipolytica. In a further embodiment, a host cell of the invention is S. cerevisiae. In another embodiment, a host cell of the invention is Pichia pastoris.

The present invention also relates to libraries comprising a heterogeneous population of antibodies or a fragment thereof that may be displayed on the extracellular surface of the plasma membrane of a cell (e.g., yeast cell), referred to herein as an “antibody library of the invention”. In one embodiment, an antibody library of the invention may comprise a heterogeneous population of heavy chain variable regions. In another embodiment, an antibody library of the invention may comprise a heterogeneous population of light chain variable regions. In yet another embodiment, an antibody library of the invention may comprise a heterogeneous population of single chain antigen binding domains, including but not limited to scFv domains. In certain embodiments, the library may comprise a heterogeneous population of single chain antibodies each fused to an Fc region. In a further embodiment, an antibody library of the invention may comprise a heterogeneous population of Fc regions, including variant Fc regions.

Persons skilled in the art will appreciate that an antibody or fragment thereof may comprise a heavy chain variable region, a light chain variable region and an Fc region, any one of which, or any combination of which, may constitute a heterogeneous population within an antibody library of the invention.

In one embodiment, an antibody library of the invention is a library of full length antibodies, wherein said antibody library of the invention comprises a heterogeneous population of heavy chain variable regions and/or a heterogeneous population of light chain variable regions. In another embodiment, an antibody library of the invention is a library of antibody fragments, wherein said antibody library of the invention comprises a heterogeneous population of heavy chain variable regions and/or a heterogeneous population of light chain variable regions. In yet another embodiment, an antibody library of the invention comprises a heterogeneous population of single chain antigen binding domains, including but not limited to scFv domains. In certain embodiments, the library comprises a heterogeneous population of single chain antibodies each fused to an Fc region. In a further embodiment, an antibody library of the invention is a library of full length antibodies or Fc fusion proteins, wherein said antibody library of the invention comprises a heterogeneous population of Fc regions, including variant Fc regions. In another embodiment, an antibody library of the invention is a library of antibody fragments, wherein said antibody library of the invention comprises a heterogeneous population of Fc regions, including variant Fc regions.

The present invention also relates to libraries comprising polynucleotides encoding a heterogeneous population of antibodies or a fragment thereof that may be displayed on the extracellular surface of the plasma membrane of a cell (e.g., yeast cell), referred to herein as a “polynucleotide library of the invention”. In one embodiment, a polynucleotide library of the invention may comprise polynucleotides encoding antibodies or a fragment thereof comprising a heterogeneous population of heavy chain variable regions. In another embodiment, a polynucleotide library of the invention may comprise polynucleotides encoding antibodies or a fragment thereof comprising a heterogeneous population of light chain variable regions. In yet another embodiment, a polynucleotide library of the invention may comprise polynucleotides encoding a heterogenous population of single chain antigen binding domains, including but not limited to scFv domains. In certain embodiments, the library comprises polynucleotides encoding a heterogeneous population of single chain antibodies each fused to an Fc region. In a further embodiment, a polynucleotide library of the invention may comprise polynucleotides encoding antibodies or a fragment thereof comprising a heterogeneous population of Fc regions, including variant Fc regions.

Persons skilled in the art will appreciate that a polynucleotide encoding an antibody or fragment thereof may encode a heavy chain variable region, a light chain variable region and an Fc region, any one of which, or any combination of which, may constitute a heterogeneous population within a polynucleotide library of the invention.

In one embodiment, a polynucleotide library of the invention is a library of polynucleotides each encoding a full length antibody, wherein said polynucleotide library of the invention comprises polynucleotides encoding a heterogeneous population of heavy chain variable regions and/or a heterogeneous population of light chain variable regions. In another embodiment, a polynucleotide library of the invention is a library of polynucleotides each encoding an antibody fragment, wherein said polynucleotide library of the invention comprises polynucleotides encoding a heterogeneous population of heavy chain variable regions and/or a heterogeneous population of light chain variable regions. In still another embodiment, a polynucleotide library of the invention is a library of polynucleotides encoding single chain antigen binding domains, including but not limited to scFv domains. In certain embodiments, a polynucleotide library of the invention is a library of polynucleotides encoding single chain antibodies each fused to an Fc region. In a further embodiment, a polynucleotide library of the invention is a library of polynucleotides each encoding a full length antibody, wherein said polynucleotide library of the invention comprises polynucleotides encoding a heterogeneous population of Fc regions, including variant Fc regions. In another embodiment, a polynucleotide library of the invention is a library polynucleotides each encoding an antibody fragment, wherein said polynucleotide library of the invention comprises polynucleotides encoding a heterogeneous population of Fc regions, including variant Fc regions. In yet another embodiment, a polynucleotide library of the invention is a library polynucleotides encoding single antibody domains wherein said polynucleotide library of the invention comprises polynucleotides encoding a heterogeneous population of Fc regions, including variant Fc regions.

In one embodiment, a vector of the invention comprises a member of a polynucleotide library of the invention. Accordingly, the present invention also provides a population of vectors each comprising a member of a polynucleotide library of the invention.

In one embodiment, a population of host cells of the invention comprise a polynucleotide library of the invention. A population of host cells comprising the library is also referred to herein as a “host cell library of the invention.” It will be understood by one of skill in the art that each member of the population of host cells (i.e., each host cell) generally comprises a limited number of members of a polynucleotide library of the invention. In one embodiment, each host cell comprises between one and six members of a polynucleotide library of the invention. Accordingly, it is contemplated that each host cell will express no more than one, or no more than two, or no more than three antibodies of the invention on the extracellular surface of the plasma membrane.

In one embodiment, a population of host cells comprise a library of the invention, wherein said host cells are selected from the group consisting of: Saccharomyces cerevisiae, Hansenula polymorpha, Kluyveromyces lactis, Pichia pastoris, Schizosaccharomyces pombe, or Yarrowia lipolytica. In a specific embodiment, a population of host cells comprise a library of the invention, wherein said host cell is Pichia pastoris. In a another embodiment, a population of host cells comprise a library of the invention, wherein said host cell is Saccharomyces cerevisiae.

The invention also provides methods of screening a library of the invention. The invention also provides methods of screening that facilitate the identification of an antibody or a fragment thereof with desired characterisitcs. In one embodiment, a method of screening a library allows the identification of an antibody or a fragment thereof that binds a specific antigen. In one embodiment, a method of screening a library allows the identification of an antibody or a fragment thereof having an altered binding for a specific antigen. In one embodiment, a method of screening a library allows the identification of an antibody or fragment having an altered binding for effector molecules (e.g., FcγRs and/or C1q).

The present invention provides a method for selecting host cells comprising an antibody or a fragment thereof with desirable binding characteristics wherein said method comprises: a) introduction into host cells a library of polynucleotides encoding an antibody or a fragment thereof that may be displayed on the extracellular surface of the plasma membrane; b) culturing host cells comprising the library to allow expression and display on the extracellular surface of the plasma membrane each antibody or a fragment thereof; c) contacting the host cells with an antibody binding reagent (e.g., antigen, FcγRs, C1q); and d) isolating the host cells comprising a plasma membrane displayed antibody or a fragment thereof that binds to the antibody binding reagent. The present invention further provides methods for 1) recovering nucleic acids from the isolated host cells; 2) amplifying nucleic acids encoding at least one antibody variable region from the nucleic acids; 3) inserting the amplified nucleic acids into a second vector, wherein said second vector, with the inserted nucleic acids, encodes a secreted soluble antibody and 4) transforming a host cell with said second vector.

The present invention also provides methods for screening antibodies based on antibody dependent cell-mediated cytotoxicity (ADCC) effect. In one embodiment, a library of the invention comprises antibody Fc variants.

### 5.1 Antibodies

Essentially all types of antibodies may be utilized in accordance with the invention. These include, but are not limited to, synthetic antibodies, monoclonal antibodies, recombinantly produced antibodies, intrabodies, multispecific antibodies, diabodies, bispecific antibodies, human antibodies, humanized antibodies, chimeric antibodies, synthetic antibodies, single-chain Fvs (scFv), Fab fragments, F(ab′) fragments, disulfide-linked Fvs (sdFv), and anti-idiotypic (anti-Id) antibodies, epitope-binding fragments of any of the above and Fc-fusion of any of the above. Antibodies used in the methods of the present invention include immunoglobulin molecules and immunologically active portions of immunoglobulin molecules. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

Antibodies or antibody fragments may be from any animal origin including birds and mammals (e.g., human, murine, donkey, sheep, rabbit, goat, guinea pig, camel, horse, or chicken). In one embodiment, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes. Antibodies or antibody fragments used in accordance with the present invention may be monospecific, bispecific, trispecific or of greater multispecificity. Multispecific antibodies may specifically bind to different epitopes of desired target molecule or may specifically bind to both the target molecule as well as a heterologous epitope, such as a heterologous polypeptide or solid support material. See, e.g., PCT Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553. The present invention may also be practiced with single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; PCT Publication Nos. WO 94/04678 and WO 94/25591; U.S. Pat. No. 6,005,079).

Embodiments of the invention include antibodies that bind to any target. Any antibody known in the art may be engineered to be displayed on the extracellular surface of the plasma membrane. Accordingly, any antibody may be used to generate a library of variants with altered binding characteristics. The variation between members of a library may be restricted to the heavy chain variable region, the light chain variable region, the Fc region, or any combination thereof.

Antibodies may be from any species, be chimeric antibodies or humanized antibodies. In one embodiment, the antibodies are human antibodies. In one embodiment, the antibodies are humanized antibodies.

It is also contemplated that a library of plasma membrane displayed variants may be generated from an antibody already described in the art including but not limited to anti-fluorescein monoclonal antibody, 4-4-20 (Kranz et al., 1982 J. Biol. Chem. 257(12): 6987-6995), a humanized anti-TAG72 antibody (CC49) (Sha et al., 1994 Cancer Biother. 9(4): 341-9), an antibody that specifically bind an Eph Receptor including, but not limited to those disclosed in PCT Publication Nos. WO 04/014292, WO 03/094859 and U.S. patent application Ser. No. 10/863,729, antibodies that specifically bind Integrin αᵥβ₃ including, but not limited to, LM609 (Scripps), the murine monoclonal LM609 (PCT Publication WO 89/015155 and U.S. Pat. No. 5,753,230); the humanized monoclonal antibody MEDI-522 (a.k.a. VITAXIN®, MedImmune, Inc., Gaithersburg, Md.; Wu et al., 1998, PNAS USA 95(11): 6037-6042; PCT Publications WO 90/33919 and WO 00/78815), an antibody against interferon alpha as disclosed in WO/2005/05059106, an antibody against the interferon receptor 1 as disclosed in WO/2006/059106, Erbitux™ (also known as IMC-C225) (ImClone Systems Inc.), a chimerized monoclonal antibody against EGFR; HERCEPTIN® (Trastuzumab) (Genentech, CA) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection. Other examples are a humanized anti-CD 18 F(ab′)₂ (Genentech); CDP860 which is a humanized anti-CD18 F(ab′)₂ (Celltech, UK); PRO542 which is an anti-HIV gp120 antibody fused with CD4 (Progenics/Genzyme Transgenics); C14 which is an anti-CD14 antibody (ICOS Pharm); a humanized anti-VEGF IgG1 antibody (Genentech); OVAREX™ which is a murine anti-CA 125 antibody (Altarex); PANOREX™ which is a murine anti-17-IA cell surface antigen IgG2a antibody (Glaxo Wellcome/Centocor); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); Smart ID10 which is a humanized anti-HLA antibody (Protein Design Lab); ONCOLYM™ (Lym-1) is a radiolabelled murine anti-HLA DR antibody (Techniclone); anti-CD11a is a humanized IgG1 antibody (Genetech/Xoma); ICM3 is a humanized anti-ICAM3 antibody (ICOS Pharm); IDEC-114 is a primatized anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-β4β7 antibody (LeukoSite/Genentech); OrthoClone OKT4A is a humanized anti-CD4 IgG antibody (Ortho Biotech); ANTOVAT™ is a humanized anti-CD40L IgG antibody (Biogen); ANTEGREN™ is a humanized anti-VLA-4 IgG antibody (Elan); MDX-33 is a human anti-CD64 (FcγR) antibody (Medarex/Centeon); rhuMab-E25 is a humanized anti-IgE IgG1 antibody (Genentech/Norvartis/Tanox Biosystems); IDEC-152 is a primatized anti-CD23 antibody (IDEC Pharm); ABX-CBL is a murine anti CD-147 IgM antibody (Abgenix); BTI-322 is a rat anti-CD2 IgG antibody (Medimmune/Bio Transplant); Orthoclone/OKT3 is a murine anti-CD3 IgG2a antibody (ortho Biotech); SIMULECT™ is a chimeric anti-CD25 IgG1 antibody (Novartis Pharm); LDP-01 is a humanized anti-β₂-integrin IgG antibody (LeukoSite); Anti-LFA-1 is a murine anti CD18 F(ab′)₂ (Pasteur-Merieux/Immunotech); CAT-152 is a human anti-TGF-β₂ antibody (Cambridge Ab Tech); and Corsevin M is a chimeric anti-Factor VII antibody (Centocor).

Additional antibodies which may be utilized in accordance with the present invention may specifically bind a cancer or tumor antigen for example, including, but not limited to, KS 1/4 pan-carcinoma antigen (Perez and Walker, 1990, J. Immunol. 142: 3662-3667; Bumal, 1988, Hybridoma 7(4): 407-415), ovarian carcinoma antigen (CA125) (Yu et al., 1991, Cancer Res. 51(2): 468-475), prostatic acid phosphate (Tailor et al., 1990, Nucl. Acids Res. 18(16): 4928), prostate specific antigen (Henttu and Vihko, 1989, Biochem. Biophys. Res. Comm. 160(2): 903-910; Israeli et al., 1993, Cancer Res. 53: 227-230), melanoma-associated antigen p97 (Estin et al., 1989, J. Natl. Cancer Instil. 81(6): 445-446), melanoma antigen gp75 (Vijayasardahl et al., 1990, J. Exp. Med. 171(4): 1375-1380), high molecular weight melanoma antigen (HMW-MAA) (Natali et al., 1987, Cancer 59: 55-63; Mittelman et al., 1990, J. Clin. Invest. 86: 2136-2144), prostate specific membrane antigen, carcinoembryonic antigen (CEA) (Foon et al., 1994, Proc. Am. Soc. Clin. Oncol. 13: 294), polymorphic epithelial mucin antigen, human milk fat globule antigen, colorectal tumor-associated antigens such as: CEA, TAG-72 (Yokata et al., 1992, Cancer Res. 52: 3402-3408), CO17-1A (Ragnhammar et al., 1993, Int. J. Cancer 53: 751-758); GICA 19-9 (Herlyn et al., 1982, J. Clin. Immunol. 2: 135), CTA-1 and LEA, Burkitt's lymphoma antigen-38.13, CD19 (Ghetie et al., 1994, Blood 83: 1329-1336), human B-lymphoma antigen-CD20 (Reff et al., 1994, Blood 83:435-445), CD33 (Sgouros et al., 1993, J. Nucl. Med. 34:422-430), melanoma specific antigens such as ganglioside GD2 (Saleh et al., 1993, J. Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12: 1036-1044), ganglioside GM3 (Hoon et al., 1993, Cancer Res. 53: 5244-5250), tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46: 3917-3923), antigens of fibrosarcoma, human leukemia T cell antigen-Gp37 (Bhattacharya-Chatterjee et al., 1988, J. of Immun. 141:1398-1403), neoglycoprotein, sphingolipids, breast cancer antigen such as EGFR (Epidermal growth factor receptor), HER2 antigen (p185^{HER2}), polymorphic epithelial mucin (PEM) (Hilkens et al., 1992, Trends in Bio. Chem. Sci. 17:359), malignant human lymphocyte antigen-APO-1 (Bernhard et al., 1989, Science 245: 301-304), differentiation antigen (Feizi, 1985, Nature 314: 53-57) such as I antigen found in fetal erythrocytes, primary endoderm I antigen found in adult erythrocytes, preimplantation embryos, I(Ma) found in gastric adenocarcinomas, M18, M39 found in breast epithelium, SSEA-1 found in myeloid cells, VEP8, VEP9, Myl, VIM-D5, D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma antigen, CO-514 (blood group Le^{a}) found in adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, G_{M2}, OFA-2, G_{D2}, and M1:22:25:8 found in embryonal carcinoma cells, and SSEA-3 and SSEA-4 found in 4 to 8-cell stage embryos. In one embodiment, the antigen is a T cell receptor derived peptide from a Cutaneous Tcell Lymphoma (see, Edelson, 1998, The Cancer Journal 4:62).

### 5.2 Transmembrane Domains

The present invention relates to an antibody or a fragment thereof that may be displayed on the extracellular surface of the yeast plasma membrane, referred to herein as an “antibody of the invention” and like terms. In certain embodiments, an antibody of the invention may comprise a heavy chain or a fragment thereof and a light chain or a fragment thereof, wherein either the heavy chain or light chain comprises a transmembrane domain that targets the antibody or a fragment thereof to the extracellular surface of the yeast plasma membrane. The transmembrane domain may be derived from natural sources or may be of synthetic origin. Synthetic transmembrane domains are described in U.S. Pat. No. 7,052,906 to Lawson, et al.

Transmembrane regions of proteins are highly hydrophobic or lipophilic domains that are the proper size to span the lipid bilayer of the cellular membrane, thereby anchoring the protein in the cell membrane. They will typically, but not always, comprise 15-30 amino acids. See Chou et al. (1999 Biotechnology and Bioengineering 65(2):160-169), which describes using a number of transmembrane domains derived from different source proteins to display a wide array of proteins on the cell surface. One skilled in the art can adapt the method performed in Chou et al., 1999 to optimize or screen different transmembrane domains for use in the present invention.

A given transmembrane protein may contain a single or multiple transmembrane domains. For example, receptor tyrosine kinases, certain cytokine receptors, receptor guanylyl cyclases and receptor serine/threonine protein kinases contain a single transmembrane domain. Other proteins, for example membrane channel components and adenylyl cyclases, contain numerous transmembrane domains as do a group of cell surface receptors classified as “seven transmembrane domain” proteins, based on the shared property of having seven membrane spanning regions. Examples of receptor proteins with a transmembrane domain include, but are not limited to, insulin receptor, insulin-like growth factor receptor, human growth hormone receptor, glucose transporters, transferrin receptor, epidermal growth factor receptor, low density lipoprotein receptor, epidermal growth factor receptor, leptin receptor, and interleukin receptors (e.g. IL-1 receptor, IL-2 receptor).

Without wishing to be bound by any theory, the majority of secreted and membrane-bound proteins in eukaryotes are translocated across the endoplasmic reticulum membrane concurrently with their translation (Wicker and Lodish, Science 230:400 (1985); Verner and Schatz, Science 241:1307 (1988); Hartmann et al., Proc. Nat'l Acad. Sci. USA 86:5786 (1989); Matlack et al., Cell 92:381 (1998)). In the first step of this co-translational translocation process, an N-terminal hydrophobic segment of the nascent polypeptide, the “signal sequence,” forms a complex with a signal recognition particle (SRP), that is subsequently anchored to the endoplasmic reticulum membrane via interaction between the SRP and the membrane bound SRP receptor. As a result of these interactions, the signal sequence traverses the endoplasmic reticulum membrane and the rest of the nascent polypeptide chain begins to pass through the translocation apparatus into the endoplasmic reticulum lumen. At the end of the transfer process, the signal peptide is cleaved off of the translocated polypeptide chain by a dedicated protease leading to the disassembly of the SRP receptor/SRP/signal peptide complex.

Single pass transmembrane proteins may be inserted into the plasma membrane via one of the following two basic mechanisms. In case of type I transmembrane domain proteins, a signal sequence located at the N terminus of the nascent polypeptide initiates translocation across the membrane as described above. Translocation stops once the transmembrane domain is inserted into the membrane. The mature membrane anchored protein is released from the translocation machinery by cleaving off the signal sequence peptide. The insertion process of a type I transmembrane domain protein always leads to an orientation where the C terminus of the mature protein is located in the cytosol. Consequently, the N terminus of a plasma membrane bound type I transmembrane domain is located in the extracellular space. In case of type II and III transmembrane proteins, translocation is initiated by an internal “signal anchor domain” that serves both as a transmembrane domain and an internal signal sequence element. The “signal anchor domain” is recognized by the SRP and is brought to the endoplasmic reticulum membrane via SRP-SRP receptor interaction. The “signal anchor domain” inserts itself into the membrane to initiate protein translocation and remains membrane bound at the end of the translocation process. The signal anchor domain is not cleaved; it is part of the mature membrane bound protein. The orientation of the initial interaction between the “signal anchor domain” and the protein translocation machinery determines the final orientation of the mature transmembrane protein. Type II and III transmembrane domain proteins have their N and C terminus, respectively, in the cytosol. Consequently, plasma membrane bound type II and III transmembrane domains have their C and N terminus, respectively, in the extracellular space.

In one embodiment, the transmembrane domain is from a type I membrane protein. In another embodiment, the transmembrane domain is a type II or type III signal anchor domain.

Described herein are examples of transmembrane domains, but the transmembrane domain of the fusion proteins of the invention can be any amino acid sequence that will span the plasma membrane and can anchor other domains to the membrane. Characteristics of transmembrane domains include generally consecutive hydrophobic amino acids that may be followed by charged amino acids. Therefore, upon analysis of the amino acid sequence of a particular protein, the localization and number of transmembrane domains within the protein may be predicted by those skilled in art. A transmembrane domain may comprise hydrophobic regions or amphipathic regions. Hydrophobic regions contain hydrophobic amino acids, which include, but are not limited to, phenylalanine, methionine, isoleucine, leucine, valine, cysteine, tryptophan, alanine, threonine, glycine and serine and include hydrophobic alpha-helices.

Amphipathic regions may have both hydrophobic and hydrophilic amino acids and moieties and include amphipathic alpha-helices. Hydrophilic amino acids include, but are not limited to, arginine, aspartate, lysine, glutamate, asparagine, glutamine, histidine, tyrosine and proline. Transmembrane domains that form stable alpha helices have been previously described in the art.

Essentially any transmembrane domain is compatible with the present invention. Transmembrane domains include, but are not limited to, those from: a member of the tumor necrosis factor receptor superfamily, CD30, platelet derived growth factor receptor (PDGFR, e.g. amino acids 514-562 of human PDGFR; Chestnut et al. 1996 J Immunological Methods 193:17-27; also see Gronwald et al. 1988 PNAS 85:3435); nerve growth factor receptor, Murine B7-1 (Freeman et al. 1991 J Exp Med 174:625-631), asialoglycoprotein receptor H1 subunit (ASGPR; Speiss et al. 1985 J Biol Chem 260:1979-1982), CD27, CD40, CD120a, CD120b, CD80 (Freeman et al. 1989 J Immunol 143:2714-22) lymphotoxin beta receptor, galactosyltransferase (E.G. GenBank accession number AF155582), sialyl transferase (E.G. GenBank accession number NM-003032), aspartyl transferase 1 (Asp1; e.g. GenBank accession number AF200342), aspartyl transferase 2 (Asp2; e.g. GenBank accession number NM-012104), syntaxin 6 (e.g. GenBank accession number NM-005819), ubiquitin, dopamine receptor, insulin B chain, acetylglucosaminyl transferase (e.g. GenBank accession number NM-002406), APP (e.g. GenBank accession number A33292), a G-protein coupled receptor, thrombomodulin (Suzuki et al. 1987 EMBO J. 6, 1891) and TRAIL receptor. In one embodiment, the transmembrane domain is from a human protein. For the purposes of the present invention all or part of a transmembrane domain from a protein may be utilized. In specific embodiments, the transmembrane domain is residues 454-477 of the Asp2, residues 598-661 of APP (e.g., of APP 695), residues 4-27 of galactosyltransferase, residues 470-492 of Asp1, residues 10-33 of sialyltransferase, residues 7-29 of acetylglucosaminyl transferase or residues 261-298 of syntaxin 6. Examples of transmembrane domains are also described in Patent Publications WO 03/104415 and US20040126859. In one embodiment, the transmembrane domain is derived from a human protein.

In one embodiment, a cell surface displayed antibody or a fragment thereof of the current invention comprises the transmembrane domain of thrombomodulin having an amino acid sequence of SEQ ID NO:2 or a functional fragment thereof. In another embodiment, a cell surface displayed antibody or a fragment thereof of the current invention comprises a transmembrane domain that is at least 70%, or at least 80%, or at least 90%, or at least 95%, or at least 97%, or at least 99% identical to SEQ ID NO:2.

In one embodiment, a cell surface displayed antibody or a fragment thereof of the current invention comprises the transmembrane domain of Axl2p having an amino acid sequence of SEQ ID NO:4 or a functional fragment thereof. In another embodiment, a cell surface displayed antibody or a fragment thereof of the current invention comprises a transmembrane domain that is at least 70%, or at least 80%, or at least 90%, or at least 95%, or at least 97%, or at least 99% identical to SEQ ID NO:4.

In one embodiment, a cell surface displayed antibody or a fragment thereof of the current invention comprises the transmembrane domain of Swp1p having an amino acid sequence of SEQ ID NO:6 or a functional fragment thereof. In another embodiment, a cell surface displayed antibody or a fragment thereof of the current invention comprises a transmembrane domain that is at least 70%, or at least 80%, or at least 90%, or at least 95%, or at least 97%, or at least 99% identical to SEQ ID NO:6.

### 5.3 GPI Anchors

A wide range of cell-surface proteins, including enzymes, coat proteins, surface antigens, and adhesion molecules, are attached to plasma membranes via GPI anchors (Burikofer et al. 2002 FASEB J 15:545). GPI is a post-translationally added lipid anchor; therefore, unlike conventional polypeptide anchors which have different transmembrane domains and connect to specific cytoplasmic extensions, GPI anchors use a common lipid structure to attach to the membrane, which is irrespective of the proteins linked with it (Englund et al., Annul Rev. Biochem. 62:121 (1993)). GPI anchor domains have been identified for many proteins (for example, see Cares et al., Science 243:1196 (1989)). The GPI anchor signals have been successfully engineered onto the C-terminus of other proteins, and these GPI anchored proteins are coated on the cell surface and are functional. (Anderson et al., P.N.A.S. 93:5894 (1996); Brunschwig et al., J. Immunother. 22:390 (1999)). GPI anchors are proposed to function in protein targeting, transmembrane signaling, and in the uptake of small molecules (endocytosis). GPI anchors of plasma membrane proteins are present in eukaryotes from protozoa and fungi to vertebrates. For examples of GPI anchor domains, which may be utilized in the present invention, see Doering, T. L. et al. (1990) J. Biol. Chem. 265:611-614; McConville, M. J. et al. (1993) Biochem. J. 294:305-324; and PCT Publication WO 03/017944).

In Saccharomyces cerevisiae, GPI-associated proteins have several common structural characteristics: a signal sequence for secretion in the N terminus and a GPI signal for attachment to GPI in the C terminus (see, Hamada et al., J Bacteriol., 181(13):3886-3889 (1999)). The GPI signal itself has three domains: the region containing the GPI attachment site (the ω site) plus the first and second amino acids downstream of the ω site, a spacer of 5 to 10 amino acids, and a hydrophobic stretch of 10 to 15 amino acids. A protein containing the GPI signal is cleaved at the w site, and the resulting carboxy terminus of the protein is covalently bound to a GPI moiety. This reaction occurs in the endoplasmic reticulum. Being associated with membranes by means of the GPI moiety, GPI-attached proteins are then transported to the cell surface and remain on the plasma membrane as GPI-anchored proteins; however, some of them are further processed. They are incorporated into the cell wall by detaching themselves from the GPI moiety and then by linking themselves to β-1,6-glucan of the cell wall. These different processes suggest that each GPI-attached protein has a signal for selecting either to be incorporated into the cell wall or to remain on the plasma membrane. Two kinds of amino acid sequences in the region upstream of the ω site (the ω-minus region) have been proposed as being responsible for the selection: dibasic residues for remaining on the plasma membrane and specific amino acid residues at sites 4 or 5 and 2 amino acids upstream of the ω site (ω-4/5 and ω-2 sites, respectively) for incorporation into the cell wall.

Essentially any GPI anchor domain is compatible with the present invention. Non-limiting examples of S. cerevisiae GPI anchors useful for the present invention are listed in Table 1.

TABLE 1
GPI anchor domains

| | | SEQ |
|---|---|---|
| | | ID |
| ORF/Gene | GPI anchor sequence | NO: |
| YIL011W/YIB1 | EKSTNSSSSATSKNAGAAMDMGFFSAG | 7 |
| | VGAAIAGAAAMLL | |
| YOL030W/GAS5 | SLLKSAASATSSSQSSSKSKGAAGIIEIPLI | 8 |
| | FRALAELYNLVL | |
| YDR055W | SSGASSSSSKSSKGNAAIMAPIGQTTPLV | 9 |
| | GLLTAIIMSIM | |
| YBR078w/ECM33 | AQANVSASASSSSSSSKKSKGAAPELVP | 10 |
| | ATSFMGVVAAVGVALL | |
| YNL190W | GPGEKARKNNAAPGPSNFNSIKLFGVTA | 11 |
| | GSAAVAGALLLL | |
| YDR144C/YAP2 | SSTGMLSPTSSSSTRKENGGHNLNPPFFA | 12 |
| | RFITAIFHHI | |
| YIR039C/YAP6 | SSFSSSGGSSESTTKKQNAGYKYRSSFSF | 13 |
| | SLLSFISYFLL | |
| YLR194C | YKSTVNGKVASVMSNSTNGATAGTHIA | 14 |
| | YGAGAFAVGALLL | |
| YLR120C/YAP3 | SGNLTTSTASATSTSSKRNVGDHIVPSLP | 15 |
| | LTLISLLFAFI | |
| YDR522C/SPS2 | GKNGAKSQGSSKKMENSAPKNIFIDAFK | 16 |
| | MSVYAVFTVLFSIIF | |
| YMR215W/GAS3 | TGSSSASSSSKSKGVGNIVNVSFSQSGYL | 17 |
| | ALFAGLISALL | |
| YMR008C/PLB1 | ASGSSTHKKNAGNALVNYSNLNTNTFIG | 18 |
| | VLSVISAVFGLI | |
| YOL132W/GAS4 | EDADEDKDDLKRKHRNSASISGPLLPLG | 19 |
| | LCLLFFTFSLFF | |

### 5.4 Polynucleotides

The current invention also relates to polynucleotides encoding an antibody or a fragment thereof that may be displayed on the extracellular surface of the plasma membrane, referred to herein as a “polynucleotides of the invention”. In one embodiment, a polynucleotide of the invention comprises two operatively linked coding regions, wherein the first coding region encodes an antibody polypeptide (e.g., a heavy chain or a fragment thereof) and the second coding region encodes an amino acid sequence that targets the antibody for display on the extracellular surface of the plasma membrane (e.g., transmembrane domain). In one embodiment, a polynucleotide of the invention comprises two operatively linked coding regions wherein the first and second coding regions are linked in-frame so as one polypeptide is formed during translation. In a specific embodiment, the first polynucleotide encodes an immunoglobulin heavy chain or a fragment thereof and the second polynucleotide encodes the transmembrane domain of human thrombomodulin (SEQ ID NO:1)

In one embodiment, a polynucleotide of the invention comprises two operatively linked coding regions, wherein said coding regions are immediately juxtaposed. In a further embodiment, a polynucleotide of the invention comprises two operatively linked coding regions wherein said coding regions are separated by at least one codon. In one embodiment, said at least one codon separating said first end second coding regions encodes a linker or spacer sequence separating the antibody polypeptide (e.g., heavy chain) and the plasma membrane targeting polypeptide (e.g., transmembrane domain).

### 5.5 Vectors

The present invention further relates to vectors comprising polynucleotides encoding an antibody or a fragment thereof that may be displayed on the extracellular surface of the plasma membrane, referred to herein as a “vector of the invention”. In certain embodiments, a vector of the invention is operable in a host cell to direct the expression and display of an antibody or a fragment thereof on the extracellular surface of the plasma membrane. In one embodiment, a vector of the invention is operable in a yeast cell to direct the expression and plasma membrane display of an antibody or a fragment thereof.

A wide range of vectors are known in the art and available commercially which meet various requirements for recombinant gene expression in yeast. Most yeast vectors are shuttle vectors, which contain sequences permitting them to be selected and propagated in E. coli, thus allowing for convenient amplification and subsequent alteration in vitro. The most common yeast shuttle vectors originated from pBR322. They contain an origin of replication promoting high copy-number maintenance in E. coli (e.g., ColE1 origin of replication), and a selectable antibiotic marker (e.g., the β-lactamase gene, tetracycline resistance gene conferring resistance to, respectively, ampicillin and tetracycline). Specific yeast shuttle vectors include, but are not limited to those described in U.S. Pat. Nos. 5,866,404 and 6,897,353. Additional yeast vectors useful for practicing the inventions described herein, fore example, but not limited to, the expression of a cell surface displayed antibody in a Pichic pastoris host cell, are described in U.S. Pat. Nos. 5,707,828, 6,730,499, U.S. Patent Publication No. 2006/0270041, and PCT Publication Nos. 05/040395, 04/04165 and 02/31178.

All yeast vectors contain marker genes that allow selection of transformants containing the desired plasmid. Examples of the most commonly used yeast marker genes include, but are not limited to, URA3, HIS3, LEU2, TRP1 and LYS2, which complement specific auxotrophic mutations in the host cell, such as ura3-52, his3-Δ1, leu2-Δ1, trp1-Δ1 and lys2-201. The URA3 and LYS2 yeast marker genes have an additional advantage because they allow the use of both positive and negative selection schemes. Selectable marker genes conferring dominant drug resistant phenotype to the yeast host cell, such as the hph and nat genes conferring resistance to hygromycin B and nourseothricin, respectively (see, Sato et al., Yeast 22:583-591 (2005)) may also be used.

Most currently used yeast shuttle vectors fall into one of the following three broad categories: (i) integrative vectors, (ii) autonomously replicating high copy-number vectors, or (iii) autonomously replicating low copy-number vectors. Integrative vectors do not replicate autonomously, but integrate into the genome at low frequencies by homologous recombination. The site of integration can be targeted by cutting the yeast segment in an integrative with a restriction endonuclease and transforming the yeast strain with the linearized plasmid. Integrative vectors typically integrate as a single copy. However multiple integration do occur at low frequencies, a property that can be used to construct stable strains overexpressing specific genes. Strains transformed with integrative vectors are extremely stable, even in the absence of selective pressure. Examples for integrative vectors include, but are not limited to pRS304 (ATCC), pRS305 (ATCC) and pRS306 (ATCC) (see, Sikorski and Hieter, Genetics, 122(1):19-27, (1989)).

Autonomously replicating high copy-number vectors are episomal vectors based on the 2 μm yeast plasmid. Their replication is governed by the 2 μm replication origin (ori) resulting in high copy-number and high frequency of transformation. The vectors contain either a full copy of the 2 μm plasmid, or, as with most of these kinds of vectors, a region which encompasses the ori and the REP3 gene. The REP3 gene is required in cis to the ori for mediating the action of the trans-acting REP1 and REP2 genes which encode products that promote partitioning of the plasmid between cells at division. Therefore, 2 μm plasmids containing the region encompassing only ori and REP3 must be propagated in cir⁺ hosts containing the native 2 μm plasmid. Examples for 2 μm vectors include, but are not limited to, pYES2/GS (Invitrogen), pRegal (Invitrogen), pBridge (Clontech).

Autonomously replicating low copy-number vectors contain a yeast centromere sequence (CEN), and an autonomously replicating sequence (ARS). These vectors are typically present at 1 to 3 copies per cell, and are lost in approximately 10⁻² cells per generation without selective pressure. The ARS sequences are believed to correspond to the natural replication origins of yeast chromosomes. The CEN function is dependent on three conserved domains, designated I, II, and III; all three of these elements are required for mitotic stabilization of the vectors. The stability and low copy-number of autonomously replicating low copy-number vectors make them a good choice for library construction. ARS1, which is in close proximity to TRP1, is one of the most commonly used ARS elements. CEN3, CEN4 and CEN11 are examples of commonly used centromeres. Examples for autonomously replicating low copy-number vectors include, but are not limited to pYD1 (Invitrogen), pRS314 (ATCC), pRS315 (ATCC) and pRS316 (ATCC) (see, Sikorski and Hieter, Genetics, 122(1):19-27, (1989)).

To achieve effective cellular expression of a plasma membrane displayed antibody, the polynucleotides encoding each antibody polypeptide are operatively linked to a transcriptional promoter to regulate expression of the polypeptide chains. The effective promoter must be functional in the eukaryotic host cell. The promoter can be a constitutive promoter or an inducible promoter. In order to achieve balanced expression and to ensure simultaneous induction of expression, a vector construct that utilizes the same promoter for each chain is preferred. Promoters useful in the present invention and functional linkages thereto are numerous and well known in the art, and the present invention is not limited by the use thereof. Promoters specifically contemplated include those useful in yeast expression vectors, such as the AOX1 promoter, galactose inducible promoters (pGAL1, pGAL1-10, pGal4, and pGal10), phosphoglycerate kinase promoter (pPGK), cytochrome c promoter (pCYC1), and alcohol dehydrogenase I promoter (pADH1).

In one embodiment, a vector of the invention comprises the pGAL1 galactose inducible promoter.

Polynucleotides encoding a plasma membrane displayed antibody polypeptide are operatively linked to a transcription terminator sequence to facilitate proper mRNA processing. In eukaryotes, transcription termination by RNA polymerase II is linked to the process of cleavage and polyadenylation of the precursor RNA. In yeast cells, transcription termination occurs within 100 nucleotides downstream of polyA sites, consistent with the short intergenic regions found in this organism. Yeast regulatory elements required for precursor RNA cleavage and polyA formation consist of three components: (1) the efficiency element that enhances the efficiency of a downstream positioning element, (2) the positioning element that positions the polyA site, and (3) the actual polyA site. Some of the sequence elements regulating 3′ RNA processing are also directly involved in transcription termination. Transcription termination elements useful in the present invention and functional linkages thereto are numerous and well known in the art, and the present invention is not limited by the use thereof. Examples of transcription termination elements include, but are not limited to, the 3′ flank sequences of several yeast genes, such as CYC1, ADH1, ARO4, TRP1, ACT1 AND YPT1.

In one embodiment, a vector of the invention comprises a transcription terminator element derived from the CYC1 genomic locus (SEQ ID NO:20). In another embodiment, a cell surface displayed antibody or a fragment thereof of the current invention comprises a transcription terminator element that is at least 70%, or at least 80%, or at least 90%, or at least 95%, or at least 97%, or at least 99% identical to SEQ ID NO:20.

In one embodiment, a vector of the invention comprises a transcription terminator element derived from the alpha factor locus (SEQ ID NO:21:). In another embodiment, a cell surface displayed antibody or a fragment thereof of the current invention comprises a transcription terminator element that is at least 70%, or at least 80%, or at least 90%, or at least 95%, or at least 97%, or at least 99% identical to SEQ ID NO:21.

To facilitate cellular processing and transport to the surface of an antibody of the invention, a polynucleotide encoding said antibody may be operatively linked to a polynucleotide element encoding a signal sequence peptide. In certain embodiments, an antibody of the invention may comprise a heavy chain or a fragment thereof and a light chain or a fragment thereof, wherein the nascent form of heavy chain and/or light chain comprises a signal sequence that targets the antibody or a fragment thereof for membrane insertion.

Signal sequences are small N terminal peptide elements that target a nascent polypeptide for transport into or across a cellular membrane. Within the eukaryotic proteome there are a number of different signal sequences that can target a protein for transport across a membrane into a specific subcellular compartment (e.g., mitochondria). Specifically contemplated are signal sequences that target a nascent polypeptide to the endoplasmic reticulum (ER) for secretion across or insertion into the plasma membrane. Signal sequences for the endoplasmic reticulum (ER) are approximately 15-30 amino acids long peptide elements located at the N terminus of the nascent polypeptide chain. They usually comprise a block of 5-10 hydrophobic amino acids. The secondary structure and general biochemical characteristics of a signal sequence appear to be more important for its function than the primary sequence itself. Signal sequences for the ER from different proteins of a eukaryotic genus frequently function interchangeably.

In one embodiment, individual subunits of an antibody of the invention are expressed in a eukaryotic host and transported to the endoplasmic reticulum (ER) for assembly and transport to the plasma membrane for extracellular display. In one embodiment, the nascent form of each individual polypeptide subunit of an antibody of the current invention comprises an ER specific signal sequence at its N terminus. The signal sequence can be the same or different for the nascent form of each subunit of an antibody of the invention. The signal sequence can be native to the host or heterologous, as long as it is operable to effect extracellular transport or membrane insertion of the polypeptide to which it is fused. Numerous signal sequences operable in the present invention are known to persons skilled in the art (e.g., Aga2p signal sequence, Mfα1 prepro-peptide, Mfα1 pre-peptide, acid phosphatase Pho5, Invertase SUC2 signal sequence). The signal sequences may be derived from native secretory proteins of the host cell, for example the eukaryotic signal sequences of α-mating factor of yeast, α-agglutinin of yeast, invertase of Saccharomyces, inulinase of Kluyveromyces, and the signal peptide of the Aga2p subunit of yeast a-agglutinin. In one embodiment, the signal sequence at the amino terminus of the nascent polypeptide is cleaved during post-translational processing of the protein. In one embodiment, a native signal sequence is retained.

In one embodiment, a cell surface displayed antibody or a fragment thereof of the current invention comprises in its nascent form the signal sequence of Aga2p having an amino acid sequence of SEQ ID NO:23 or a functional fragment thereof. In another embodiment, a cell surface displayed antibody or a fragment thereof of the current invention comprises in its nascent form a signal sequence that is at least 70%, or at least 80%, or at least 90%, or at least 95%, or at least 97%, or at least 99% identical to SEQ ID NO:23.

In one embodiment, a cell surface displayed antibody or a fragment thereof of the current invention comprises in its nascent form the signal sequence of the α-mating factor having an amino acid sequence of SEQ ID NO:25 or a functional fragment thereof. In another embodiment, a cell surface displayed antibody or a fragment thereof of the current invention comprises in its nascent form a signal sequence that is at least 70%, or at least 80%, or at least 90%, or at least 95%, or at least 97%, or at least 99% identical to SEQ ID NO:25.

In one embodiment, a vector of the invention is an expression vector operable in a host cell to direct the expression of both an antibody heavy chain or a fragment thereof and an antibody light chain or a fragment thereof. In one embodiment, an antibody expression vector may contain two separate expression cassettes: one for the heavy chain and one for the light chain. The two cassettes may comprise the same or different transcriptional regulatory elements (e.g., promoter and transcription terminator). When different transcriptional regulatory elements are used, care should be taken that the expression levels of heavy and light chain polypeptides are balanced. In another embodiment, polynucleotides encoding the heavy and light chain polypeptides are expressed from a single promoter and transcribed into a single RNA molecule. A number of different molecular techniques known in the art may be used to translate such a polycistronic RNA message into separate heavy and light chain polypeptides. Useful techniques include, but are not limited to, the use of an IRES element, trans-splicing ribozyme, and inteins. IRES (internal ribosomal entry site) is a sequence element derived from the 5′ untranslated regions of certain viral and cellular genes that allows for cap-independent translation of protein synthesis. The use of an IRES elements operative in a wide variety of eukaryotic cells including, but not limited to yeast has been described in U.S. Pat. Nos. 6,376,745, and 6,933,378. Trans-splicing ribozymes are RNA-based catalysts capable of splicing RNA sequences from one transcript specifically into a separate target transcript. In doing so, a chimeric mRNA can be produced that is functional as mRNA or encodes a protein to be expressed in the target cells (see, e.g., Ayre et al., Nucleic Acids Research, Vol. 30(24):e141 (2002); U.S. Patent Publication 2006/0246422A1). Inteins are internal portions of protein sequences that are post-translationally excised while the flanking regions are spliced together, making an additional protein product. Modified inteins had been described that promote the excision of an intein from a polyprotein but prevent the ligation reactions normally associated with protein splicing (see, U.S. Pat. No. 7,026,526). Additional methods are known in the art, for example the use of a host cell protease cleavable peptide linker to release heavy and light chain from a single nascent polypeptide chain (see, e.g., U.S. Patent Publication No. 20060252096A1).

In one embodiment, a vector of the invention is a set of two vectors wherein a first vector comprises a polynucleotide encoding a heavy chain of an antibody or a fragment thereof and a second vector comprises a polynucleotide encoding a light chain of an antibody or a fragment thereof, wherein said antibody or a fragment thereof a first and second vector comprises a polynucleotide encoding a first and second, respectively, polypeptide chain of an antibody or a fragment thereof that may be displayed on the extracellular surface of the plasma membrane. To achieve effective cellular expression of an antibody, the polynucleotides encoding each of the antibody chains are, linked to a transcriptional promoter to regulate expression of the polypeptide chains. The effective promoter must be functional in the host cell. The promoter can be a constitutive promoter or an inducible promoter. In order to achieve balanced expression and to ensure simultaneous induction of expression, a vector set that utilizes the same promoter for each chain may be used. A number of promoters useful in the present invention are known in the art. Promoters contemplated include those useful in yeast expression vectors, such as galactose inducible promoters (pGAL1, pGAL1-10, pGal4, and pGal10), phosphoglycerate kinase promoter (pPGK), cytochrome c promoter (pCYC1), and alcohol dehydrogenase I promoter (pADH1). In one embodiment, a vector of the invention is a set of a first vector and a second vector wherein said first and second vectors comprise the galactose inducible promoter pGAL1 operatively linked to a polynucleotide of the invention.

In one embodiment, to achieve optimal cellular expression of an antibody, polynucleotides encoding each of the antibody chains are operatively linked to a transcription terminator element. In one embodiment, a vector of the invention is a set of a first vector and second vector wherein said first vector comprises a transcription terminator element derived from the CYC1 genomic locus (SEQ ID NO:20), and said second vector comprises a transcription terminator element derived from the alpha mating factor locus (SEQ ID NO:21). In another embodiment, a vector of the invention is a set of a first vector and second vector wherein said first vector comprises a transcription terminator element that is at least 70%, or at least 80%, or at least 90%, or at least 95%, or at least 97%, or at least 99% identical to SEQ ID NO:20, and said second vector comprises a transcription terminator element that is at least 70%, or at least 80%, or at least 90%, or at least 95%, or at least 97%, or at least 99% identical to SEQ ID NO:21.

It is contemplated that each of the polynucleotides encoding an antibody chain is also linked to a signal sequence. An effective signal sequence must be functional in the host cell. Polynucleotides encoding the antibody chains are typically directly linked, in frame (either immediately adjacent to the polynucleotide or optionally linked via a linker or spacer sequence), to a polynucleotide encoding a signal sequence, thus generating a polypeptide chain-signal sequence peptide fusion protein. In certain embodiments, each chain of a multi-chain polypeptide is fused to a separate signal sequence peptide. The polynucleotide encoding a signal sequence peptide can be the same or different for each chain of the multi-chain polypeptide. The signal sequence can be native to the host or heterologous, as long as it is operable to effect extracellular transport of the polypeptide to which it is fused. In one embodiment, a vector of the invention is a set of a first and second vector wherein said first vector comprises a polynucleotide encoding the signal sequence of Aga2p having an amino acid sequence of SEQ ID NO:23 or a functional fragment thereof and said second vector comprises a polynucleotide encoding the signal sequence of the α-mating factor having an amino acid sequence of SEQ ID NO:25 or a functional fragment thereof. In another embodiment, a vector of the invention is a set of a first vector and second vector wherein said first vector comprises a polynucleotide encoding a signal sequence that is at least 70%, or at least 80%, or at least 90%, or at least 95%, or at least 97%, or at least 99% identical to SEQ ID NO:23, and said second vector comprises a polynucleotide encoding a signal sequence that is at least 70%, or at least 80%, or at least 90%, or at least 95%, or at least 97%, or at least 99% identical to SEQ ID NO:25.

An antibody of the invention may be displayed on the extracellular surface of the plasma membrane. Display on the plasma membrane is achieved by fusing at least one polypeptide chain of an antibody of the invention with a transmembrane domain. More than one polypeptide chain of an antibody of the invention may be fused with a transmembrane domain, but only one chain need be fused to achieve plasma membrane display. Polynucleotides encoding an antibody chains are typically directly linked, in frame (either immediately adjacent to the polynucleotide or optionally linked via a linker or spacer sequence), to a polynucleotide encoding a transmembrane domain, thus generating an antibody chain-transmembrane domain fusion protein. In one embodiment, a vector of the invention is a set of two vectors, wherein at least one vector comprises a polynucleotide encoding a transmembrane domain operatively linked to a polynucleotide encoding a polypeptide chain of an antibody of the invention (e.g., a heavy chain or fragment thereof). In one embodiment, a vector of the invention is a set of two vectors, wherein at least one vector comprises a polynucleotide encoding the transmembrane domain of thrombomodulin having an amino acid sequence of SEQ ID NO:2 operatively linked to a polynucleotide encoding a polypeptide chain of an antibody of the invention. In one embodiment, a vector of the invention is a set of two vectors, wherein at least one vector comprises a polynucleotide encoding the transmembrane domain of Axl2p having an amino acid sequence of SEQ ID NO:4 operatively linked to a polynucleotide encoding a polypeptide chain of an antibody of the invention. In one embodiment, a vector of the invention is a set of two vectors, wherein at least one vector comprises a polynucleotide encoding the transmembrane domain of Swp1p having an amino acid sequence of SEQ ID NO:6 operatively linked to a polynucleotide encoding a polypeptide chain of an antibody of the invention (e.g., a heavy chain or a fragment thereof).

Given proper selection of expression vector components and compatible host cells, the chains of the multi-chain polypeptide (e.g., antibody or fragment thereof) will be displayed on the plasma membrane of a eukaryotic host cell. Persons skilled in the art will appreciate that this can be achieved using any of a number of variable expression vector constructs, and that the present invention is not limited thereby. The display vector itself can be constructed or modified from any of a number of genetic vectors and genetic control elements known in the art and commercially available (e.g., from Invitrogen, Clontech, Stratagene, ATCC). Essentially, a vector of the present invention encompasses any vector capable of expressing an antibody or a fragment thereof having a transmembrane domain for effective display on the extracellular surface of the plasma membrane of a eukaryotic host cell transformed with said vector.

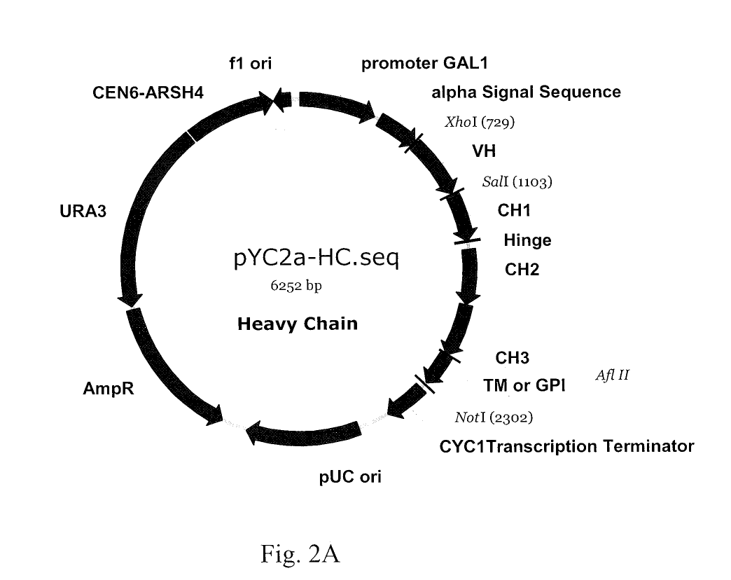

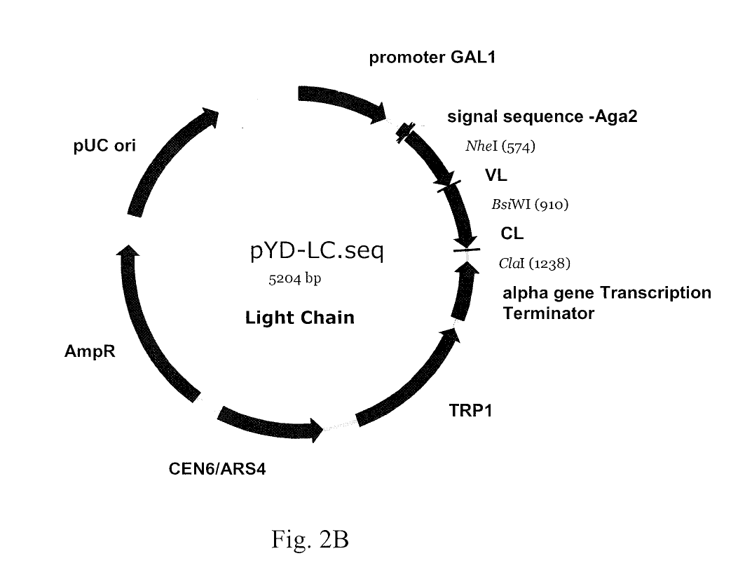

The vector pair depicted in FIGS. 2 A and B is a non-limiting example of a vector of the invention.

### 5.6 Libraries

The present invention also provides libraries comprising antibodies or a fragment thereof that may be displayed on the extracellular surface of the plasma membrane, referred to herein as an “antibody library of the invention”. In one embodiment, an antibody library of the invention may comprise a heterogeneous population of heavy chain variable regions. In another embodiment, an antibody library of the invention may comprise a heterogeneous population of light chain variable regions. In yet another embodiment, an antibody library of the invention may comprise a heterogeneous population of single chain antigen binding domains, including but not limited to scFv domains. In certain embodiments, the library may comprise a heterogeneous population of single chain antibodies each fused to an Fc region. In a further embodiment, an antibody library of the invention may comprise a heterogeneous population of Fc regions, including variant Fc regions.

Persons skilled in the art will appreciate that a heterogeneous population of sequences may exist within the context of an antibody or a fragment thereof within the library. For example, a heterogeneous population of heavy chain variable regions may exist in the context of a library of full length antibodies.

In one embodiment, an antibody library of the invention is a library of full length antibodies, wherein said antibody library of the invention comprises a heterogeneous population of heavy chain variable regions and/or a heterogeneous population of light chain variable regions. In another embodiment, an antibody library of the invention is a library of antibody fragments, wherein said antibody library of the invention comprises a heterogeneous population of heavy chain variable regions and/or a heterogeneous population of light chain variable regions. In yet another embodiment, an antibody library of the invention comprises a heterogeneous population of single chain antigen binding domains, including but not limited to scFv domains. In certain embodiments, the library comprises a heterogeneous population of single chain antibodies each fused to an Fc region. In a further embodiment, an antibody library of the invention is a library of full length antibodies, or Fc fusion proteins wherein said antibody library of the invention comprises a heterogeneous population of Fc regions, including variant Fc regions. In another embodiment, an antibody library of the invention is a library of antibody fragments, wherein said antibody library of the invention comprises a heterogeneous population of Fc regions, including variant Fc regions.

An antibody library of the invention may comprise a plurality of essentially any type of antibodies. These include, but are not limited to, synthetic antibodies, monoclonal antibodies, recombinantly produced antibodies, intrabodies, multispecific antibodies, diabodies, bispecific antibodies, human antibodies, humanized antibodies, chimeric antibodies, synthetic antibodies, single-chain Fvs (scFv), Fab fragments, F(ab′) fragments, disulfide-linked Fvs (sdFv), and anti-idiotypic (anti-Id) antibodies, epitope-binding fragments of any of the above and Fc fusions of any of the above. Antibodies may be monospecific, bispecific, trispecific or of greater multispecificity. Multispecific antibodies may specifically bind to different epitopes of desired target molecule or may specifically bind to both the target molecule as well as a heterologous epitope, such as a heterologous polypeptide or solid support material. A library of the invention may comprise a plurality of immunoglobulin molecules and an immunologically active portion of immunoglobulin molecules. The immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule.

The present invention also relates to libraries comprising polynucleotides encoding a heterogeneous population of antibodies or a fragment thereof that may be displayed on the extracellular surface of the plasma membrane, referred to herein as a “polynucleotide library of the invention”. Any polynucleotide library encoding an antibody library described herein is a polynucleotide library of the invention.

In one embodiment, a polynucleotide library of the invention may comprise polynucleotides encoding antibodies or a fragment thereof comprising a heterogeneous population of heavy chain variable regions. In another embodiment, a polynucleotide library of the invention may comprise polynucleotides encoding antibodies or a fragment thereof comprising a heterogeneous population of light chain variable regions. In a further embodiment, a polynucleotide library of the invention may comprise polynucleotides encoding antibodies or a fragment thereof comprising a heterogeneous population of Fc regions, including variant Fc regions.

Persons skilled in the art will appreciate that a polynucleotide encoding an antibody or fragment thereof may comprise a polynucleotide encoding a heavy chain variable region, a polynucleotide encoding a light chain variable region and a polynucleotide encoding an Fc region, any one of which, or any combination of which, may constitute a heterogeneous population within a polynucleotide library of the invention.

In one embodiment, a polynucleotide library of the invention is a library of polynucleotides each encoding a full length antibody, wherein said polynucleotide library of the invention comprises polynucleotides encoding a heterogeneous population of heavy chain variable regions and/or a heterogeneous population of light chain variable regions. In another embodiment, a polynucleotide library of the invention is a library of polynucleotides each encoding an antibody fragment, wherein said polynucleotide library of the invention comprises polynucleotides encoding a heterogeneous population of heavy chain variable regions and/or a heterogeneous population of light chain variable regions. In still another embodiment, a polynucleotide library of the invention is a library of polynucleotides encoding single chain antigen binding domains, including but not limited to scFv domains. In certain embodiments, a polynucleotide library of the invention is a library of polynucleotides encoding single chain antibodies each fused to an Fc region. In a further embodiment, a polynucleotide library of the invention is a library of polynucleotides each encoding a full length antibody, wherein said polynucleotide library of the invention comprises polynucleotides encoding a heterogeneous population of Fc regions, including variant Fc regions. In another embodiment, a polynucleotide library of the invention is a library polynucleotides each encoding an antibody fragment, wherein said polynucleotide library of the invention comprises polynucleotides encoding a heterogeneous population of Fc regions, including variant Fc regions.

A polynucleotide library of the present invention can be constructed by any number of methods know to those skilled in the art. Briefly, a library of polynucleotides comprising a diverse repertoire of nucleic acid sequences encoding an antibody or a fragment thereof is isolated. For example, the repertoire of nucleic acid sequences encoding an antibody heavy chain can be isolated from, for example, an antibody cDNA library, a library of cDNA molecules generated from nucleic acids (e.g., poly A+ RNA) isolated from any tissue or cell population expressing antibodies. The repertoire of coding sequences may then be amplified, for example by PCR, and cloned into a vector using standard methods known in the art (see, e.g., U.S. Patent Publication No's 2005/00048617A1, 2005/00042664A1; Wu, H., Methods Mol Biol., 207:197-212 (2003); Wu, H. & Ann, L. L., Methods Mol Biol., 207:213-33 (2003)). Libraries of antibody coding sequences are also commercially available. In one embodiment, the library is constructed using coding regions from human antibodies. In some embodiments, the library expresses at least 2, 10, 100, 10³, 10⁴, 10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹ or 10¹² different antibodies.

Once a library of polynucleotides encoding an antibody or a fragment thereof is obtained, it may be cloned into a vector of the invention. Essentially any methods known for cloning nucleic acids into a vector can be utilized. These methods include, but are not limited to, restriction enzyme digestion and ligation, SOEing PCR or recombination (see, e.g., Horton, et al., 1989, Gene, 77, 61-68; Wu, H., Methods Mol Biol., 207:197-212 (2003); Wu, H. & Ann, L. L., Methods Mol Biol., 207:213-33 (2003)).

A library of the invention can be derived from any source. It can be a large library isolated from a vertebrate (e.g., human, mouse, rat, donkey, goat, cat, dog, chicken, camel) and essentially represent a complete repertoire of antibodies or a fragment thereof from a single specimen or a population of the source organism. In one embodiment, a library is representative of the entire repertoire of a single human or a human population. In one embodiment, a library is representative of the entire repertoire of a mouse or a group of mice. A library may be isolated from a immunologically naüve individual. A library may also be isolated from a vertebrate that has been previously immunized with the antigen of interest. The library therefore may be enriched for antibodies that bind the antigen of interest. In another embodiment, a phage display antibody library is screened against the antigen of interest. The antibody library for the present invention is then created from those phage that express an antibody that binds the antigen of interest. Again, the library is enriched for antibodies that bind the antigen of interest. In still another embodiment, an antibody library for the present invention is generated from a library of humanized antibody fragments. Humanized antibody fragments may be generated by any method known to one of skill in the art including, but not limited to, framework shuffling (e.g., PCT Publication WO 05/042743) and low homology humanization (e.g., PCT Publication WO 05/035575). In another embodiment, the library is a mutant CDR library derived from an antibody that binds the antigen of interest. A mutant CDR library is a library coding for antibodies that are CDR mutations of a parent antibody's CDR sequences. Mutant CDR include, but are not limited to, libraries created by mutating CDR amino acids that are determined to be contact residues by crystallographic studies (e.g., Dall'Acqua et al. 1996 Biochemistry 35:9667-76); libraries created by retaining one native CDR (e.g. the one believed to have the highest binding efficiency) and combining with a library of CDRs in place of the other 5 CDRs (e.g., Rader et al., 1998, PNAS 95:8910-15); a library created by “CDR walking” (e.g., Yang et al., 1995, J Mol Biol 254:392-403; and a library created by a method of separately mutating each CDR of a parental antibody (e.g., Wu et al., 1998, PNAS 95:6037-42). Therefore, any library of antibodies may be used in accordance with the present invention to express the library on the plasma membrane. In one embodiment, the library is an affinity maturation library derived from a parental antibody.

In another embodiment, a phage display antibody library is screened against the antigen of interest. The antibody library for the present invention is then created from those phage that express an antibody that binds the antigen of interest. In one embodiment, both the heavy chain and light chain variable regions from each selected phage are cloned into a vector of the invention, wherein each vector encodes a heavy and light chain. Therefore, the same heavy and light chain combinations are maintained. In another embodiment, the heavy chain and light chain variable regions are isolated and combined in a random matter. Therefore, theoretically the library comprises every combination of each pre-selected heavy chain variable sequence with each pre-selected light chain sequence. For example, if the initial phage screen resulted in 1,000 unique phage and antibody sequences, a library comprised of every combination of each pre-selected heavy chain variable sequence with each pre-selected light chain sequence would now account for 10⁶ possible unique antibody sequences that would be cloned into a viral vector of the invention. This method creates an even more diverse repertoire than the initial phage library. Additionally, while the phage display method is limited to certain antibody fragments, the method described herein allows a screen of whole antibodies. A population of antibody fragments selected in a first phage display screen and converted into full length antibodies may therefore be subjected to a second round of screening. Examples of phage display methods that can be used to make the antibody libraries of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

For further details and methods for cloning antibody libraries see e.g., PCT Patent Publication WO 2005/063817; WO 95/15393; U.S. Patent Publication No's 2005/00048617A1, 2005/00042664A1; Wu, H., Methods Mol Biol., 207:197-212 (2003); Wu, H. & Ann, L. L., Methods Mol Biol., 207:213-33 (2003); Higuchi et al. 1997 J Immunological Methods 202:193-204.

A antibody library of the invention may comprise Fc variant antibodies. In one embodiment, a library of Fc variants comprises antibodies having the same variable regions or Fab regions but different Fc regions. In a specific embodiment, a library of Fc variants may contain variants of the hinge domain, CH3 domain, CH2 domain or any combination thereof.

Methods for constructing Fc variants and Fc variant antibody libraries are know in the art. For examples, see Patent Publication Nos. WO 05/0037000; WO 06/023420; and WO 06/023403.

A polynucleotide library of the invention comprises polynucleotides encoding an antibody or a fragment thereof that may be displayed on the extracellular surface of the plasma membrane. A polynucleotide library of the invention may comprise polynucleotides encoding a plurality of heavy chains or a fragment thereof, it may further comprise polynucleotides encoding a plurality of light chains or a fragment thereof, and it may further comprise polynucleotides encoding a plurality of variant Fc regions or a fragment thereof. It will be apparent to one skilled in the art that a polynucleotide encoding an antibody or a fragment thereof may comprise sequence elements encoding a heavy chain variable region, a light chain variable region and a Fc region. Any one of these sequence elements, or any combination of these sequence elements may encode a heterogeneous population of antibody polypeptides within the context of a polynucleotide library of the invention.

A polynucleotide library of the invention may be cloned into a vector to generate a vector library of the invention. The mode of introducing the polynucleotides into a vector includes any of the applicable methods of recombinant DNA technology known in the art. A vector may comprise a single vector or a set of two vectors. In one embodiment, a vector library of the invention is generated by introducing a polynucleotide library of the invention into a vector; wherein said vector is a single vector; and wherein said single vector comprises polynucleotides encoding a heavy chain or a fragment thereof and a light chain or a fragment thereof. In another embodiment, a vector library of the invention is generated by introducing a polynucleotide library of the invention into a vector; wherein said vector is a set of a first vector and a second vector; and wherein said first vector comprises polynucleotides encoding a heavy chain or a fragment thereof, and said second vector comprises polynucleotides encoding a light chain or a fragment thereof

### 5.7 Host Cells

The present invention also relates to host cells comprising an antibody or a fragment thereof that may be displayed on the extracellular surface of the plasma membrane, referred to herein as a “host cell of the invention” and like terms. A host cell of the invention is any eukaryotic cell capable of expressing an antibody or a fragment thereof that may be displayed on the extracellular surface of the host cell plasma membrane. A host cell of the invention can be any eukaryotic cell, of any genotype, differentiated or undifferentiated, unicellular or multi-cellular, depending on the practitioner's particular interest and requirements. In one embodiment, the host cell is an undifferentiated, unicellular, haploid or diploid cellular organism. In another embodiment, a host cell of the invention is a Fungus. In a further embodiment, a host cell of the invention is part of the phylum Ascomycota. In one embodiment, a host cell of the invention is of the genera Neurospora, Saccharomyces, Pichia, Hansenula, Schizosaccharomyces, Kluyveromyces, Yarrowia, Debaryomyces, and Candida. In another embodiment, a host cell of the invention is selected from the group consisting of: Saccharomyces cerevisiae, Hansenula polymorpha, Kluyveromyces lactis, Pichia pastoris, Schizosaccharomyces pombe and Yarrowia lipolytica. In a specific embodiment, a host cell of the invention is Saccharomyces cerevisiae. In a further embodiment, a host cell of the invention is Pichia pastoris.

The basic life cycle of eukaryotic cells involves an alternation between diploid and haploid states. For many fungi, the haploid stage of the life cycle predominates. Importantly, the natural recombination and re-mixing of genetic material that results from the cellular fusion of separate haploid cells to produce a diploid cell is a powerful process that can be utilized in biological research. In a particular embodiment, a host cell of the invention is a cell suitable for cell fusion. For example, yeast cells of opposite mating type can be “mated” to produce fused diploid cells. In addition, yeast protoplasts or spheroplasts suitable for cell fusion are also suitable eukaryotic host cells for the purposes of the invention.

A host cell comprising an antibody of the invention may be generated by transforming a host cell with a polynucleotide encoding an antibody or a fragment thereof that may be displayed on the extracellular surface of the plasma membrane. The polynucleotide encoding an antibody or a fragment thereof can be introduced into the host cell via one or more vectors. The mode of introducing the vector(s) into the host cell includes any of the methods for introducing genetic material into a cell known in the art. Examples for such methods include, but are not limited to, electroporation, microinjection, viral transfer, ballistic insertion, and the like.

In one embodiment, a host cell of the invention comprises an antibody or fragment thereof that may be displayed on the extracellular surface of the plasma membrane.

In one embodiment, a host cell of the invention comprises a vector of the invention wherein said vector is a single vector comprising a polynucleotide sequence encoding a heavy chain of an antibody or a fragment thereof and a light chain of an antibody or a fragment thereof; wherein said antibody or a fragment thereof may be displayed on the extracellular surface of the plasma membrane. In another embodiment, a host cell of the invention comprises a vector of the invention wherein said vector is a vector set of a first vector and a second vector. In one embodiment, a host cell of the invention comprises a set of two vectors; wherein a first vector comprises a polynucleotide encoding a heavy chain of an antibody or a fragment thereof, and a second vector comprises a polynucleotide encoding a light chain of an antibody or a fragment thereof; wherein said antibody or a fragment thereof may be displayed on the extracellular surface of the plasma membrane.

In one embodiment, a host cell of the invention is a diploid cell comprising a vector of the invention, wherein said vector is a single vector comprising a polynucleotide sequence encoding a heavy chain of an antibody or a fragment thereof and a light chain of an antibody or a fragment thereof; wherein said antibody or a fragment thereof may be displayed on the extracellular surface of the plasma membrane.

In one embodiment, a host cell of the invention is a diploid cell. In one embodiment, a host cell of the invention is a diploid cell comprising a set of two vectors; wherein a first vector comprises a polynucleotide encoding a heavy chain of an antibody or a fragment thereof, and a second vector comprises a polynucleotide encoding a light chain of an antibody or a fragment thereof; wherein said antibody or a fragment thereof may be displayed on the extracellular surface of the plasma membrane.

In one embodiment, a host cell of the invention is a diploid cell that was generated by cell fusion. In one embodiment, a diploid host cell of the invention is generated by fusing a first haploid host cell and a second haploid host cell, wherein said first and second haploid host cells are of opposite mating types; and wherein said first haploid host cell comprises a polynucleotides encoding a heavy chain or a fragment thereof of an antibody of the invention; and wherein said second haploid host cell comprises a polynucleotide encoding a light chain or a fragment thereof of an antibody of the invention. For further details and methods see U.S. Patent Publication Nos. 2003/0186374, 2006/0270041, and PCT Publication No. 05/040395.

A polynucleotide library of the invention may be introduced into a population of host cells to generate a host cell library of the invention. Advantageously, a polynucleotide library of the invention may be introduced into a host cell population by transforming said host cell population with a vector library of the invention. The mode of introducing a vector library of the invention into a population of host cells includes any of the methods for introducing genetic material into a cell known in the art. Examples for such methods include, but are not limited to electroporation, microinjection, viral transfer, ballistic insertion, and the like (e.g., Gietz, D. et al., Nucleic Acids Res., 20:1425 (1992)). In a specific embodiment, a polynucleotide library of the invention may be introduced into a population of host cells by utilizing the host cell's gap repair mechanism (see, e.g., Swers J S et al. Biochem Biophys Res Commun. 350(3):508-13 (2006)).

In one embodiment, a vector library of the invention is generated by introducing a polynucleotide library of the invention into a vector; wherein said vector is a single vector; and wherein said single vector comprises polynucleotides encoding a heavy chain or a fragment thereof and a light chain or a fragment thereof. Said vector library of the invention may be introduced into a host cell population via any of the methods for introducing genetic material into a cell known in the art. Examples for such methods include, but are not limited to electroporation, microinjection, viral transfer, ballistic insertion, and the like (e.g., Gietz, D. et al., Nucleic Acids Res., 20:1425 (1992)). In a specific embodiment, a polynucleotide library of the invention may be introduced into a population of host cells by utilizing the host cell's gap repair mechanism (see, e.g., Swers J S et al. Biochem Biophys Res Commun. 350(3):508-13 (2006)).

In one embodiment, a vector library of the invention is generated by introducing a polynucleotide library of the invention into a vector; wherein said vector is a set of a first vector and a second vector; and wherein said first vector comprises polynucleotides encoding a heterogeneous population of heavy chains or a fragment thereof, and said second vector comprises polynucleotides encoding a heterogeneous population of light chains or a fragment thereof. Said vector library of the invention may be introduced into a host cell population via fusion of first haploid host cell population and a second haploid host cell population, wherein said first and second haploid host cell populations are of opposite mating types; and wherein said first haploid host cell population comprises polynucleotides encoding a heterogeneous population of heavy chains or a fragment thereof; and wherein said second haploid host cell population comprises polynucleotides encoding a heterogeneous population of light chains or a fragment thereof. The natural recombination and re-mixing of genetic material that results from the cellular fusion of separate haploid cells to produce a diploid cell is a powerful process that can be utilized in biological research. For example, a library of full length antibodies with increased diversity may be generated by recombining via cell fusion a heavy chain only library of lower diversity with a light chain only library of lower diversity. For further details and methods see U.S. Patent Publication No. 2003/0186374, 2006/0270041, and PCT Publication No. 05/040395.

### 5.8 Methods of Screening

The invention also provides methods of screening a library comprising antibodies or a fragment thereof that may be displayed on the extracellular surface of the plasma membrane, hereinafter referred to as a “screening method of the invention”. In one embodiment, a screening method of the invention allows the identification of an antibody or a fragment thereof that binds a specific antigen. In one embodiment, a screening method of the invention allows the identification of an antibody or a fragment thereof having an altered binding for a specific antigen. In one embodiment, a screening method of the invention allows the identification of an antibody or fragment having an altered binding for effector molecules (e.g., FcγRs and/or C1q).

The present invention provides a method for selecting host cells comprising an antibody or a fragment thereof with desirable binding characteristics wherein said method comprises: a) introduction into host cells of a library of polynucleotides encoding an antibody or a fragment thereof that may be displayed on the extracellular surface of the plasma membrane of a cell (e.g., yeast cell); b) culturing said host cells comprising the library (also referred to herein as a “host cell library of the invention”) to allow expression and display on the extracellular surface of the plasma membrane each antibody or a fragment thereof; c) contacting said host cells with an antibody binding reagent (also referred to herein as an “antibody ligand” or simply as a “ligand”); and d) isolating the host cells comprising a plasma membrane displayed antibody or a fragment thereof that binds to the antibody binding reagent. Antibody binding agents include, but are not limited to antigens, FcγRs and C1q. Desirable binding characteristics include, but are not limited to, binding to a specific antigen, increased binding to a specific antigen, reduced binding to a specific antigen. Desirable binding characteristics further include, but are not limited to, binding to an effector molecule (e.g., C1q, FcγRI, FcγRII, FcγRIIIA), increased binding to an effector molecule, and reduced binding to an effector molecule. The present invention further provides methods for 1) recovering nucleic acids from the isolated host cells; 2) amplifying nucleic acids encoding at least one antibody variable region from the nucleic acids; 3) inserting the amplified nucleic acids into a second vector, wherein said second vector, with the inserted nucleic acids, encodes a secreted soluble antibody and 4) transforming a host cell with said second vector.

It will be understood by one of skill in the art that each host cell of the host cell library generally comprises a limited number of polynucleotides encoding an antibody or a fragment thereof that may be displayed on the extracellular surface of the plasma membrane of a cell. In one embodiment, each host cell comprises between one and six polynucleotides encoding an antibody or a fragment thereof that may be displayed on the extracellular surface of the plasma membrane of a cell. Accordingly, it is contemplated that each host cell will express no more than one, or no more than two, or no more than three antibodies of the invention on the extracellular surface of the plasma membrane.

In one embodiment, a host cell of the invention is a yeast cell. Yeast cells are protected by a cell envelope consisting of three major constituents (inside out): the plasma membrane, the periplasmic space, and the cell wall. The cell envelope plays a major role in controlling the osmotic and permeability properties of the cell. The cell wall, comprising 15-25% of the dry cell mass, acts as the primary gatekeeper by preventing large molecular weight compounds (e.g., antibodies) from entering the periplasmic space. Main structural components of the cell wall are β-glucans, chitin and mannose containing glycoproteins (mannoproteins). The porosity of the yeast cell wall is mainly controlled by the mannoproteins (see, Zlotnik et al., J. Bacteriology, 159:1018-1026 (1984); De Nobel et al., J. Gen. Microbiol. 135:2077-2084 (1989)). An antibody of the invention displayed on the extracellular surface of the plasma membrane, and thus located in the periplasmic space, is shielded from the outside environment by the cell wall and may be prevented from interacting with some antibody binding reagents such as a protein antigen. It will be apparent to one of skill in the art that a screening method of the invention exploiting differential interaction between an antibody and an interacting ligand (e.g., antigen, FcγRs, C1q) may not be feasible in the presence of an intact cell wall. To facilitate the screening procedure, methods are provided herein to increase the porosity of the cell wall allowing access to the antibody displayed in the periplasmic space.

The porosity of the yeast cell wall may be increased by a number of different methods (e.g., the use of mutant host cells, chemical inhibitors of cell wall synthesis, cell wall degrading enzyme treatment of host cells) and the present invention is not limited by the use thereof. In one embodiment, a host cell of the invention is a yeast cell comprising a mutation that leads to the increased porosity of the cell wall. In a specific embodiment, a host cell of the invention comprises a mutation that reduces or eliminates the function of a gene selected from the group consisting of: mnn1, mnn2, mnn9 and orthologues of mnn1, mnn2, mnn9; wherein said mutation leads to the increased porosity of the cell wall.

In one embodiment, a method of the invention comprises the step of culturing a yeast host cell in the presence of a chemical inhibitor, wherein the activity of the chemical inhibitor leads to the formation of a cell wall with increased porosity. In a specific embodiment, said chemical inhibitor is tunicamycin. In one embodiment, a method of the invention comprises the step of treating a host cell of the invention with an enzyme, wherein said enzyme treatment increases the porosity of the cell wall. In a specific embodiment, said enzyme is selected from the group consisting of: lyticase and zymolase. In one embodiment, a method for selecting yeast host cells having an antibody or a fragment thereof with desirable binding characteristics comprises: a) introduction into yeast host cells a library of polynucleotides encoding an antibody or a fragment thereof that may be displayed on the extracellular surface of the plasma membrane of a cell (e.g., yeast cell); b) culturing yeast host cells comprising the library to allow expression and display on the extracellular surface of the plasma membrane each antibody or a fragment thereof; c) contacting said yeast host cells with an enzyme that renders the cell wall sufficiently porous to make it permeable for an antibody ligand; d) contacting said yeast host cells with an antibody binding reagent (e.g., antigen; FcγRs, C1q); and e) isolating the host cells comprising a plasma membrane displayed antibody or a fragment thereof that binds to the antibody binding reagent. The method of the invention may further incorporate the additional steps of 1) recovering nucleic acids from the isolated yeast host cells; 2) amplifying nucleic acids encoding at least one antibody variable region from the nucleic acids; 3) inserting the amplified nucleic acids into a second vector, wherein said second vector, with the inserted nucleic acids, encodes a secreted soluble antibody and 4) transforming a yeast host cell with said second vector.

Once a host cell library of the invention is generated, it may be screened against at least one antigen of interest. A host cell library of the invention may also be screened to identify an Fc variant with desirable characteristics. It will be appreciated by those of skill in the art that numerous variations for screening may be made without departing from the invention as described herein.

A host cell library of the invention is cultured to allow expression of the antibody library of the invention, which may be displayed on the cell surface. The cells are then screened to identify and select the ones expressing an antibody with the desired ligand (e.g., antigen, FcγRs, C1q) binding properties or other desired characteristics. The cells can be screened and selected by methods described herein and those known to one skilled in the art. Cells expressing antibodies with the desired properties are then selected by separation from the other cells.

The screening and selection step can be accomplished using any of a variety of techniques known in the art including those described herein. Most frequently used techniques comprise the steps of: a) incubation of a host cell library of the invention with the ligand of interest (e.g., antigen, FcγRs, C1q) under conditions that allow the specific binding of the ligand of interest to an antibody but prevent nonspecific association of the ligand of interest with an antibody or the host cell; b) detection of host cell bound ligand of interest; c) separation of host cells with bound ligand of interest from host cells without bound ligand of interest. Useful binding conditions include, but are not limited to the use of PBS buffer (pH 7.6) with 0.1% Triton-X 100. In certain embodiments, the antibody binding reagent of interest is tagged to facilitate detection and/or separation as described infra.

The ligand of interest may be detected using any one of a large number of methods know to one skilled in the art and the current invention is not limited by the use thereof (e.g., U.S. Pat. Nos. 5,994,519, 6,180,336, 6,489,123). The ligand of interest may be detected with a specific binding reagent (e.g. a ligand specific antibody or a fragment thereof, a ligand specific aptamer) that is itself labeled to aid detection (e.g., fluorescently labeled reagent, magnetic bead conjugated reagent, Sepharose bead conjugated reagent). The ligand may be modified to aid direct detection (e.g. fluorescently labeled ligand, GFP protein fusion ligand). The ligand may be tagged or modified to aid indirect detection (e.g., biotinylated ligand, HA affinity tag fusion ligand, FLAG tag fusion ligand, MBP tag fusion ligand, streptavidin fusion ligand); and the ligand is detected indirectly via the use of a labeled (e.g., fluorescently labeled, magnetic bead conjugated, Sepharose bead conjugated) secondary reagent that specifically interacts with the tag or modification of the ligand (e.g., streptavidin for biotinylated ligands, anti-biotin antibody or a fragment thereof for biotinylated ligands, anti-HA antibody for HA tagged ligands, anti-FLAG antibody for FLAG tagged ligands).

In one embodiment, the ligand of interest (e.g., antigen, FcγRs, C1q) may be tagged (e.g. fluorescent marker) and used to bind to antibodies on the host cell surface; thus labeling the host cells expressing antibodies that bind to the ligand. In one embodiment the fluorescent label is selected from the group consisting of Aqua, Texas-Red, FITC, rhodamine, rhodamine derivatives, fluorescein, fluorescein derivatives, cascade blue, Cy5, phycoertythrin, GFP or a GFP derivative e.g., EGFP. Numerous fluorescent labels are known in the art and commercially available (see, e.g., Molecular Probes: Handbook of Fluorescent Probes and Research Chemicals, R. P. Haugland, 9th ed., Molecular Probes, (OR, 2004)). In one embodiment, the ligand of interest is biotin-labeled. The cells bind the ligand of interest and a label conjugated to streptavidin is used to label ligand bound cells. In one embodiment, PE-conjugated streptavidin is used. In one embodiment, the ligand of interest comprises streptavidin and labeled biotin is used as the detection reagent. In one embodiment, the ligand of interest is recombinantly produced and incorporates a peptide tag (e.g., FLAG, HIS tag). Antibodies to the peptide tag can be used to detect and sort/select for or exclude cells that bind the particular antigen.

The method for separation of host cells with specifically bound ligand of interest from host cells without bound ligand of interest may be achieved by using any one of a large number of methods know to one skilled in the art and the current invention is not limited by the use thereof. For example, in the case of a fluorescently tagged ligand, the host cells can be separated/sorted, for example, by a flow cytometer and sorted based on fluorescence. For examples, see PCT Publication Nos. WO 04/014292, WO 03/094859, WO 04/069264, WO 04/028551, WO 03/004057, WO 03/040304, WO 00/78815, WO 02/070007 and WO 03/075957, U.S. Pat. Nos. 5,795,734, 6,248,326 and 6,472,403, Pecheur et al., 2002, FASEB J. 16: 1266-1268; Almed et al., 2002, J. Histochemistry & Cytochemistry 50:1371-1379. In another embodiment, fluorescently labeled host cells are observed using a fluorescent microscope and may be isolated directly using standard micromanipulation techniques such as a fine glass pipette, micropipettor or a micromanipulator. In another embodiment, the cells can be sorted/separated using beads (e.g., U.S. Pat. Nos. 6,342,588, 5,665,582, and 4,219,411; Chestnut et al., 1996, J Immunological Methods 193:17-27). For example, an antigen can be biotinylated, and cells expressing antibodies that bind to the antigen can be isolated using streptavidin-coated magnetic beads.

More than one antigen may be utilized in the selection step, for example, if screening for antibodies that bind a first antigen but not a second antigen. In this case, a negative selection step could be carried out by sorting for cells expressing antibodies that do not bind the second antigen, followed by a positive selection step that sorts for antibodies that bind the first antigen. In one embodiment, the positive selection step is carried out prior to the negative selection step. In one embodiment, the positive and negative selection step is carried out essentially simultaneously. For example, first and second antigen is labeled with different fluorescent molecules. Both antigens are incubated together with the cells displaying the antibodies. The concentration of the antibodies may be optimized for this embodiment. The cells are then simultaneously sorted for those that bind the first, but not the second antigen (e.g., two-color FACS analysis). One skilled in the art, based on the teachings herein, can negatively and/or positively screen for binding to a multitude of antigens by employing consecutive screening/selection steps and by multi-color FACS analysis.

In some embodiments antibodies that bind to a particular cell type (target cell) can be selected. Such selections in relation to phage-displayed antibodies are described in e.g. Huts et al., 2001, Cancer Immunol. Immunother. 50:163-171. The target cells can be fixed or unfixed, which may for example, offer an opportunity to select antibodies that bind to cell surface antigens that are altered by fixation. A particular cell type can be selected with reference to the biological function to be screened/selected for in the process. An appropriate cell type would be one that antibodies with the desired biological function would be expected to bind. For example, if it is desired to isolate antibodies capable of inhibiting the proliferation of cancer cells, it would be expected that such antibodies could bind to cancer cells. Thus, it would be appropriate to initially select for antibodies that can bind to cancer cells. To select for antibodies that bind to cells, the cells displaying antibodies can be screened for binding to the target cell using conditions conducive to binding. For example, a biotin-conjugated antibody that binds to the target cells, but not to the cells expressing the antibodies, can be bound to streptavidin-coated magnetic beads. These beads are then used to immobilize the target cells. The antibody-expressing cells can be combined with the immobilized cells, and those that bind to the magnetic beads can be isolated. Selection for antibodies that bind to cells, rather than specific, known antigens, has the advantage that there is a possibility of selecting for antibodies that bind to previously unknown antigens displayed on a cell surface. Such an antigen need not be a protein and may comprise more than one cell surface molecule. A selection step for binding to a chosen kind of cells or a particular molecule can be repeated once or multiple times, for example, at least about 2, 3, 4, 5, 6, 7, or more times. If desired, two or more different pre-selection steps can be performed either simultaneously or in succession. For example, antibodies that bind to two different kinds of cancer cells can be selected.

Optionally, further refinement can be achieved by one or more negative selection steps, which can be performed either before or after the positive selection step. For example, if selecting for antibodies that bind to cancer cells, the cells displaying antibodies can be allowed to bind non-cancerous cells (e.g. as described above), and antibodies that do not bind to these cells can be retained for further testing. Such a negative selection can eliminate at least some of the antibodies that bind nonspecifically to non-target cells. Alternatively, the non-target protein(s) (e.g. unrelated or similar antigen as compared to the target antigen) is affixed to a solid support and utilized in a negative selection step to eliminate antibody expressing cells that bind to the non-target protein(s). In another example, it may be desired to isolate antibodies to a certain receptor, wherein this receptor is part of a family of receptors that have closely related structures. To increase the probability of isolating an antibody specific to this particular receptor, a negative selection step may be performed using one, some or all of the other receptors from the family. In a negative selection step, cells expressing antibodies that do not bind the non-target antigen can be retained for further testing. This selection can eliminate at least some of the antibodies that bind nonspecifically to the solid support or to a non-target protein(s). Similarly, if selecting for cells displaying antibodies that bind to a particular protein, the cells can be mixed with an unrelated or similar protein to compete for binding with the target protein.

Following a first round of selection, polynucleotides encoding the selected antibodies may be isolated and used to express the corresponding antibodies for further characterization. The positively isolated cells expressing the desired antibodies may also be expanded and subjected to another round of screening. Screening methods of the present invention may employ 1, 2, 3, 4, 5, 6, 7, 8 or more selection steps.

In one embodiment, a host cell library of the invention is a library of diploid host cells comprising a first and second vector having a first and second polynucleotide encoding a first and second chain of an antibody of the invention. In one embodiment a host cell library of the invention is a diploid host cell library that was generated via fusion of first haploid host cell population and a second haploid host cell population; wherein said first and second haploid host cell populations are of opposite mating types; and wherein said first haploid host cell population comprises polynucleotides encoding a heterogeneous population of heavy chains or a fragment thereof; and wherein said second haploid host cell population comprises polynucleotides encoding a heterogeneous population of light chains or a fragment thereof. A first round of screening is performed to isolate diploid host cells displaying an antibody on the surface of the plasma membrane with desired characteristics. DNA is isolated from the isolated host cells to recover the vectors encoding the heavy and light chains of the antibody which was displayed on the surface of the plasma membrane. The isolated DNA may optionally be amplified in E. Coli. The isolated vector DNA is used to establish a secondary host cell library comprising diploid host cells expressing effectively all possible combinations of the heavy and light chains isolated from the primary screen. The secondary library may be screened for antibodies with further improved characteristics.

Once cells comprising antibodies with the desired properties are identified in the preceding selection steps, polynucleotides encoding said antibodies can be isolated and retested to ensure that they encode antibodies with the desired biological properties. If individual transformants or pools of transformants are isolated, recombinant nucleic acids can be obtained from these for retesting. For example, if individual transformants have been isolated, nucleic acids encoding the antibodies can be purified and used to transfect mammalian cells, which can then be characterized with regards to their binding properties for the antigen. If pools of transformants have been isolated, nucleic acids encoding the antibodies from pools testing positive can be used to transform cells to generate individual transformants expressing one antibody.

Nucleic acids encoding the antibodies from these individual transformants can be used to transfect cells and antibodies can be expressed, isolated and tested for function, thereby identifying proteins or antibodies having the desired function. If individual transformants or pools of transformants have not been isolated, nucleic acids encoding the protein or at least the antibody variable regions can be obtained from the transfectants or pools of transfectants that have tested positive, for example, by amplifying the expressed antibody variable region-encoding sequences by PCR. These sequences, which may be amplified by PCR, can also then be re-inserted into a suitable vector and used to generate individual transformants. Recombinant DNA from these transformants can be used to transfect mammalian cells in order express the antibodies and to retest for function.

### 5.9 Specific Antigens and Fusion Partners of the Invention

As described above, the methods of the present invention may be applied to any antibody. For example an Fc variant library may be generated from, or a variant Fc region of the invention may be introduced into any antibody. Furthermore, a variant Fc region may be utilized to generate an Fc fusion protein. Accordingly, virtually any molecule may be targeted by and/or incorporated into an antibody and/or Fc fusion protein which may be utilized in accordance with the present invention including, but not limited to, the following list of proteins, as well as subunits, domains, motifs and epitopes belonging to the following list of proteins: renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VII, factor VIIIC, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor (TNF) proteins such as TNF-alpha, TNF-beta, TNFbeta2, TNFα, TNFalphabeta, 4-1BBL as well as members of the TNF superfamily members such as, TNF-like weak inducer of apoptosis (TWEAK), and LIGHT, B lymphocyte stimulator (BlyS); members of the TNF receptor superfamily including TNF-RI, TNF-RII, TRAIL receptor-1, CD137, Transmembrane activator and CAML interactor (TACI) and OX40L; Fas ligand (FasL); enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors such as, for example, EGFR (ErbB-1), VGFR, CTGF (connective tissue growth factor); interferons such as alpha interferon (α-IFN), beta interferon (β-IFN) and gamma interferon (γ-IFN); interferon alpha receptor (IFNAR) subunits 1 and/or 2 and other receptors such as, A1, Adenosine Receptor, Lymphotoxin Beta Receptor, BAFF-R, endothelin receptor; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor; platelet-derived growth factor (PDGF); fibroblast growth factor such as αFGF and βFGF; epidermal growth factor (EGF); transforming growth factor (TGF) such as TGF-alpha and TGF-beta, including TGF-1, TGF-2, TGF-3, TGF-4, or TGF-5; insulin-like growth factor-I and -II (IGF-I and IGF-II); des (1-3)—IGF-I (brain IGF-I), insulin-like growth factor binding proteins, keratinocyte growth factor; growth factor receptors such as, FGFR-3, IGFR, PDGFRα; CD proteins such as CD2, CD3, CD3E, CD4, CD 8, CD11, CD11a, CD14, CD16, CD18, CD19, CD20, CD22, CD23, CD25, CD27, CD27L, CD28, CD29, CD30, CD30L, CD32, CD33 (p67 protein), CD34, CD38, CD40, CD40L, CD44, CD45, CD52, CD54, CD55, CD56, CD63, CD64, CD80; CD137 and CD147; IL-2R/IL-15R Beta Subunit (CD122); erythropoietin; osteoinductive factors; immunotoxins; a bone morphogenetic protein (BMP); an interferon such as interferon-alpha, -beta, and -gamma; colony stimulating factors (CSFs), such as M-CSF, GM-CSF, and G-CSF; interleukins (ILs), e.g., IL-1 to IL-13 and IL-15, IL-18, IL-23; EPO; superoxide dismutase; T-cell receptors alpha/beta; surface membrane proteins; decay accelerating factor; viral antigen such as, for example, a portion of the AIDS envelope, e.g., gp120; transport proteins; homing receptors; addressins; regulatory proteins; chemokine family members such as the eotaxins, the MIPs, MCP-1, RANTES; cell adhesion molecules such as selectins (L-selectin, P-selectin, E-selectin) LFA-1, LFA-3, Mac 1, p150.95, VLA-1, VLA-4, ICAM-1, ICAM-3, EpCAM and VCAM, a4/p7 integrin, and Xv/p3 integrin, integrin alpha subunits such as CD49a, CD49b, CD49c, CD49d, CD49e, CD49f, alpha7, alpha8, alpha9, alphaD, CD11a, CD11b, CD51, CD11c, CD41, alphaIIb, alphaIELb; integrin beta subunits such as, CD29, CD 18, CD61, CD104, beta5, beta6, beta7 and beta8; Integrin subunit combinations including but not limited to, αVβ3, αVβ5 and αβ7; cellular ligands such as, TNF-related apoptosis-inducing ligand (TRAIL), A proliferation-inducing ligand (APRIL), B Cell Activating Factor (BAFF), a member of an apoptosis pathway; IgE; blood group antigens; flk2/flt3 receptor; obesity (OB) receptor; mp1 receptor; CTLA-4; protein C; an Eph receptor such as EphA2, EphA4, EphB2, etc.; immune system markers, receptors and ligands such as CTLA-4, T cell receptor, B7-1, B7-2, IgE, Human Leukocyte Antigen (HLA) such as HLA-DR, CBL; complement proteins such as complement receptor CR1, C1Rq and other complement factors such as C3, and C5; blood factors including tissue factor, factor VII; a glycoprotein receptor such as GpIbα, GPIIb/IIIa and CD200; and fragments of any of the above-listed polypeptides.

Also contemplated are cancer related proteins including, but not limited to, ALK receptor (pleiotrophin receptor), pleiotrophin; KS 1/4 pan-carcinoma antigen; ovarian carcinoma antigen (CA125); prostatic acid phosphate; prostate specific antigen (PSA); prostate specific membrane antigen (PSMA); melanoma-associated antigen p97; melanoma antigen gp75; high molecular weight melanoma antigen (HMW-MAA); prostate specific membrane antigen; carcinoembryonic antigen (CEA); carcinoembryonic antigen-related cell adhesion molecule (CEACAM1); cytokeratin tumor-associated antigen; human milk fat globule (HMFG) antigen; CanAg antigen; tumor-associated antigen expressing Lewis Y related carbohydrate; colorectal tumor-associated antigens such as: CEA, tumor-associated glycoprotein-72 (TAG-72), C017-1A, GICA 19-9, CTA-1 and LEA; Burkitt's lymphoma antigen-38.13; CD19; human B-lymphoma antigen-CD20; CD22; CD33; melanoma specific antigens such as ganglioside GD2, ganglioside GD3, ganglioside GM2 and ganglioside GM3; tumor-specific transplantation type cell-surface antigen (TSTA); virally-induced tumor antigens including T-antigen, DNA tumor viruses and Envelope antigens of RNA tumor viruses; oncofetal antigen-alpha-fetoprotein such as CEA of colon, 5T4 oncofetal trophoblast glycoprotein and bladder tumor oncofetal antigen; differentiation antigen such as human lung carcinoma antigens L6 and L20; antigens of fibrosarcoma; human leukemia T cell antigen-Gp37; neoglycoprotein; sphingolipids; breast cancer antigens such as EGFR (Epidermal growth factor receptor); NY-BR-16; NY-BR-16 and HER2 antigen (p185^{HER2}); Her2/neu (ErbB-2), Her3 (ErbB-3), Her4 (ErbB-4), polymorphic epithelial mucin (PEM) antigen; epithelial membrane antigen (EMA); Melanoma-associated antigen MUC18; MUC1; malignant human lymphocyte antigen-APO-1; differentiation antigen such as I antigen found in fetal erythrocytes; primary endoderm I antigen found in adult erythrocytes; preimplantation embryos; I(Ma) found in gastric adenocarcinomas; M18, M39 found in breast epithelium; SSEA-1 found in myeloid cells; VEP8; VEP9; Myl; VIM-D5; D₁56-22 found in colorectal cancer; TRA-1-85 (blood group H); SCP-1 found in testis and ovarian cancer; C14 found in colonic adenocarcinoma; F3 found in lung adenocarcinoma; AH6 found in gastric cancer; Y hapten; Le^{y} found in embryonal carcinoma cells; Colonocyte differentiation antigen found in colorectal tumors, Carbonic anhydrase IX found in renal cell carcinoma, FAPα in the stroma around numerous tumor types, Folate binding protein found in ovarian tumors, PD1; death receptor proteins, DR5; TL5 (blood group A); EGF receptor found in A431 cells; E₁ series (blood group B) found in pancreatic cancer; FC10.2 found in embryonal carcinoma cells; gastric adenocarcinoma antigen; CO-514 (blood group Le^{a}) found in Adenocarcinoma; NS-10 found in adenocarcinomas; CO-43 (blood group Le^{b}); G49 found in EGF receptor of A431 cells; MH2 (blood group ALe^{b}/Le^{y}) found in colonic adenocarcinoma; 19.9 found in colon cancer; gastric cancer mucins; T₅A₇ found in myeloid cells; R₂₄ found in melanoma; 4.2, G_{D3}, D1.1, OFA-1, G_{M2}, OFA-2, G_{D2}, and M1:22:25:8 found in embryonal carcinoma cells and SSEA-3 and SSEA-4 found in 4 to 8-cell stage embryos; Cutaneous T cell Lymphoma antigen; MART-1 antigen; Sialy Tn (STn) antigen; Anaplastic lymphoma kinase (ALK) found in large cell lymphoma; Colon cancer antigen NY-CO-45; Lung cancer antigen NY-LU-12 variant A; Adenocarcinoma antigen ART1; Paraneoplastic associated brain-testis-cancer antigen (onconeuronal antigen MA2; paraneoplastic neuronal antigen); Neuro-oncological ventral antigen 2 (NOVA2); Hepatocellular carcinoma antigen gene 520; TUMOR-ASSOCIATED ANTIGEN CO-029; Tumor-associated antigens MAGE-C1 (cancer/testis antigen CT7), MAGE-B1 (MAGE-XP antigen), MAGE-B2 (DAM6), MAGE-2, MAGE-4a, MAGE-4b and MAGE-X2; Cancer-Testis Antigen (NY-EOS-1); placental alkaline phosphatase (PLAP) and testicular PLAP-like alkaline phosphatase, transferrin receptor; Heparanase I; EphA2 associated with numerous cancers; DNA/histone H1 complexes that are found in the necrotic cores of many tumor types; amino phospholipids such as phosphatidylserine; Placental Alkaline Phosphatase (PALP); cell surface glycoproteins such as CS1, gp-3, gp4 and gp9 that are associated with numerous tumor types and fragments of any of the above-listed polypeptides.

Other exemplary proteins which may be targeted by and/or incorporated into Fc variant proteins include but not limited to the following list of proteins, as well as subunits, domains, motifs, and epitopes belonging to the following list of microbial proteins: B. anthracis proteins or toxins; human cytomegalovirus (HCMV) proteins such as, envelope glycoprotein, gB, internal matrix proteins of the virus, pp 65 and pp 150, immediate early (1E) proteins; human immunodeficiency virus (HIV) proteins such as, Gag, Pol, Vif and Nef (Vogt et al., 1995, Vaccine 13: 202-208); HIV antigens gp120 and gp160 (Achour et al., 1995, Cell. Mol. Biol. 41: 395-400; Hone et al., 1994, Dev. Biol. Stand. 82: 159-162); gp41 epitope of human immunodeficiency virus (Eckhart et al., 1996, J. Gen. Virol. 77: 2001-2008); hepatitis C virus (HCV) proteins such as, nucleocapsid protein in a secreted or a nonsecreted form, core protein (pC); E1 (pE1), E2 (pE2) (Saito et al., 1997, Gastroenterology 112: 1321-1330), NS3, NS4a, NS4b and NS5 (Chen et al., 1992, Virology 188:102-113); severe acute respiratory syndrome (SARS) corona virus proteins include but are not limited to, the S (spike) glycoprotein, small envelope protein E (the E protein), the membrane glycoprotein M (the M protein), the hemagglutinin esterase protein (the HE protein), and the nucleocapsid protein (the N-protein) See, e.g., Marra et al., “The Genome Sequence of the SARS-Associated Coronavirus,” Science Express, May 2003); Mycobacterium tuberculosis proteins such as the 30-35 kDa (a.k.a. antigen 85, alpha-antigen) that is normally a lipoglycoprotein on the cell surface, a 65-kDa heat shock protein, and a 36-kDa proline-rich antigen (Tascon et al. (1996) Nat. Med. 2: 888-92), Ag85A, Ag85b (Huygen et al., 1996, Nat. Med. 2: 893-898), 65-kDa heat shock protein, hsp65 (Tascon et al., 1996, Nat. Med. 2: 888-892), MPB/MPT51 (Miki et al., 2004, Infect. Immun. 72:2014-21), MTSP11, MTSP17 (Lim et al., 2004, FEMS Microbiol. Lett. 232:51-9 and supra); Herpes simplex virus (HSV) proteins such as gD glycoprotein, gB glycoprotein; proteins from intracellular parasites such as Leishmania include LPG, gp63 (Xu and Liew, 1994, Vaccine 12: 1534-1536; Xu and Liew, 1995, Immunology 84: 173-176), P-2 (Nylen et al., 2004, Scand. J. Immunol. 59:294-304), P-4 (Kar et al. 2000, J Biol. Chem. 275:37789-97), LACK (Kelly et al., 2003, J Exp. Med. 198:1689-98); microbial toxin proteins such as Clostridium perfringens toxin; C. difficile toxin A and B; in addition, exemplary antigen peptides of human respiratory syncytial virus (hRSV), human metapneumovirus (HMPV) and Parainfluenza virus (PIV) are detailed in: Young et al., in Patent publication WO04010935A2.

One skilled in the art will appreciate that the aforementioned lists of proteins refers not only to specific proteins and biomolecules, but the biochemical pathway or pathways that comprise them. For example, reference to CTLA-4 as a target antigen and/or fusion partner implies that the ligands and receptors that make up the T cell co-stimulatory pathway, including CTLA-4, B7-1, B7-2, CD28, and any other undiscovered ligands or receptors that bind these proteins, are also useful as target antigens and/or fusion partners. Thus, the present invention encompasses not only a specific biomolecule, but the set of proteins that interact with said biomolecule and the members of the biochemical pathway to which said biomolecule belongs. One skilled in the art will also appreciate that antibodies and/or antigen binding fragments thereof, which bind to a protein, the ligands or receptors that bind them, or other members of their corresponding biochemical pathway, may be derived by methods will known in the art, such as those described below, and that such antibodies and/or antigen binding fragments may be engineered to comprise a variant Fc region or fragment thereof including, but not limited to, those described herein. One skilled in the art will further appreciate that any of the aforementioned proteins, the ligands or receptors that bind them, or other members of their corresponding biochemical pathway, may be operably linked to a variant Fc region or fragment thereof including, but not limited to, those described herein in order to generate an Fc fusion. Thus for example, an Fc fusion that targets EGFR could be constructed by operably linking a variant Fc region to EGF, TGFα, or any other ligand, discovered or undiscovered, that binds EGFR. Accordingly, a variant Fc region could be operably linked to EGFR in order to generate an Fc fusion that binds EGF, TGFα, or any other ligand, discovered or undiscovered, that binds EGFR. Thus virtually any polypeptide, whether a ligand, receptor, or some other protein or protein domain, including but not limited to the aforementioned targets and the proteins that compose their corresponding biochemical pathways, may be utilized as a fusion partner to generate an Fc variant protein. It is contemplated that the resulting Fc variant proteins (e.g., antibodies, Fc fusions) targeting and/or incorporating one or more of the molecules listed supra are formulated in accordance with the present invention.

### 5.10 Downstream Engineering

It is contemplated that one or more of the polypeptides isolated using the screening methods of the present invention may be further modified. For example, an antibody isolated in accordance with the present invention may be modified (i.e., by the covalent attachment of any type of molecule to the antibody such that covalent attachment). For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, etc. In certain embodiments antibodies, or a fragment thereof, isolated in accordance with the present invention are fused to a bioactive molecule including, but not limited to, peptides, polypeptides, proteins, small molecules, mimetic agents, synthetic drugs, inorganic molecules, and organic molecules. In other embodiments antibodies, or a fragment thereof, isolated in accordance with the present invention are conjugated to a diagnostic, detectable or therapeutic agent. Such agents and method for conjugation are well known to one of skill in the art and are disclosed in numerous sources (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119; International Publication Nos. WO 93/15199; WO 93/15200; WO 97/33899; WO 97/34911; WO 01/77137; WO 03/075957; U.S. Patent Publications 2006/0040325).

Alternatively or optionally, the antibody, or a fragment thereof, isolated in accordance with the present invention may be fused to a polypeptide moiety. Methods for fusing or conjugating antibodies to polypeptide moieties are known in the art. See, e.g., U.S. Pat. Nos. 5,336,603; 5,622,929; 5,359,046; 5,349,053; 5,447,851, and 5,112,946; EP 307,434; EP 367,166; PCT Publications WO 96/04388 and WO 91/06570; Ashkenazi et al., 1991, PNAS USA 88:10535; Zheng et al., 1995, J Immunol 154:5590; and Vil et al., 1992, PNAS USA 89:11337; each incorporated by reference in their entireties. The fusion of an antibody, or a fragment thereof, to a moiety does not necessarily need to be direct, but may occur through linker sequences. Such linker molecules are commonly known in the art and described in Denardo et al., 1998, Clin Cancer Res 4:2483; Peterson et al., 1999, Bioconjug Chem 10:553; Zimmerman et al., 1999, Nucl Med Biol 26:943; Garnett, 2002, Adv Drug Deliv Rev 53:171.

In one embodiment, antibodies, or a fragment thereof, isolated in accordance with the present invention are recombinantly fused or chemically conjugated (including both covalent and non-covalent conjugations) to a heterologous protein or polypeptide (or a fragment thereof, preferably to a polypeptide of at least 10, at least 20, at least 30, at least 40, at least 50, at least 60, at least 70, at least 80, at least 90 or at least 100 amino acids) to generate fusion proteins. Alternatively, or optionally, antibodies, or a fragment thereof, may be used to target heterologous polypeptides to particular cell types, either in vitro or in vivo, by fusing or conjugating the antibodies to antibodies specific for particular cell surface receptors. Alternatively, an antibody can be conjugated to a second antibody to form an antibody heteroconjugate as described by Segal in U.S. Pat. No. 4,676,980.

### 5.11 Specific Embodiments

1. An antibody that may be displayed on the extracellular surface of the plasma membrane of a yeast cell.
2. The antibody of embodiment 1, comprising an amino acid sequence that targets the antibody to the extracellular surface of the plasma membrane, wherein said amino acid sequence is fused to the heavy chain or the light chain of the antibody.
3. The antibody of embodiment 2, wherein said amino acid sequence is fused to the C-terminal end of the heavy chain or the light chain of the antibody.
4. The antibody of embodiment 2, wherein said amino acid sequence comprises a transmembrane domain.
5. The antibody of embodiment 2, wherein said amino acid sequence comprises a GPI anchor domain.
6. The antibody of embodiment 4, wherein said transmembrane domain is derived from thrombomodulin, Axl2p, or Swp1p.
7. The antibody of embodiment 4, wherein said transmembrane domain comprises a polypeptide having an amino acid sequence set forth as SEQ ID NO:2, 4, or 6.
8. The antibody of embodiment 1, wherein said antibody is from an immunoglobulin type selected from the group consisting of IgA, IgE, IgM, IgD, IgY and IgG.
9. The antibody of embodiment 1, wherein said antibody is a murine antibody, a chimeric antibody, a humanized antibody or human antibody.
10. The antibody of embodiment 1, wherein said antibody is a human antibody.
11. The antibody of embodiment 1, wherein said antibody is an antigen binding fragment.
12. The antibody of embodiment 1, wherein the antigen binding fragment is selected from the group consisting of a single-chain Fv (scFv); an Fab fragment; an F(ab′) fragment; and an Fd fragment.
13. The antibody of embodiment 11 or 12, wherein said antigen binding fragment is fused to an Fc region.
14. The antibody of embodiment 13, wherein said antibody comprises an amino acid sequence that targets said antibodies to the cell surface, wherein said amino acid sequence is fused to the C-terminal end of the Fc region.
15. The antibody of embodiment 1 or 13, wherein said antibody comprises a variant Fc region.
16. The antibody of embodiment 1, wherein said antibody comprises a heavy chain variable region, a light chain variable region or both a heavy chain and a light chain variable region.
17. The antibody of embodiment 1, wherein said antibody comprises a heavy chain, a light chain or both a heavy chain and a light chain.
18. A polynucleotide encoding the antibody of any one of embodiments 1-17.
19. A vector comprising the polynucleotide of embodiment 18, wherein said vector is operable in a yeast host cell to direct the expression and the display on the extracellular surface of the plasma membrane of an antibody.
20. The vector of embodiment 19, wherein said vector is a set of two vectors, wherein a first vector comprises a polynucleotide encoding a heavy chain of an antibody and a second vector comprises a polynucleotide encoding a light chain of an antibody, wherein said antibody may be displayed on the extracellular surface of the plasma membrane.
21. The vector of embodiment 20, wherein said first vector further comprises (a) an inducible promoter, (b) a signal sequence, (c) a poly A signal and (d) a transcription termination element operatively linked to said polynucleotide encoding a heavy chain of an antibody; and wherein said second vector further comprises (a) an inducible promoter, (b) a signal sequence, (c) a poly A signal and (d) a transcription termination element operatively linked to said polynucleotide encoding a light chain of an antibody.
22. The vector of embodiment 19, wherein said vector further comprises (a) an inducible promoter, (b) a signal sequence, and (c) a poly A signal and (d) a transcription termination element operatively linked to said polynucleotide encoding a yeast expressed recombinant antibody that may be displayed on the extracellular surface of the plasma membrane.
23. The vector of embodiment 19, wherein said vector further comprises a selectable marker.
24. The vector of embodiment 20, wherein said first and/or said second vector further comprises a selectable marker.
25. The vector of embodiment 19, wherein said vector is a shuttle vector.
26. The vector of embodiment 19, wherein said vector is an autonomously replicating low copy vector, an autonomously replicating high copy vector or an integrating vector.
27. A yeast cell comprising the vector of embodiment 19.
28. The yeast cell of embodiment 27, wherein said yeast cell comprises a genetic mutation rendering the cell wall sufficiently porous to make it permeable for a protein antigen or an antibody.
29. The yeast cell of embodiment 28, wherein said yeast cell comprises a genetic mutation in mnn9 or an orthologue of mnn9.
30. The yeast cell of embodiment 27, wherein said yeast cell is of a genus selected from the group consisting of: Saccharomyces, Pichia, Hansenula, Schizosaccharomyces, Kluyveromyces, Yarrowia, Debaryomyces and Candida.
31. The yeast cell of embodiment 30,wherein said yeast cell is selected from the group consisting of: Saccharomyces cerevisiae, Hansenula polymorpha, Kluyveromyces lactis, Pichia pastoris, Schizosaccharomyces pombe and Yarrowia lipolytica.
32. The yeast cell of embodiment 31, wherein said yeast cell is Saccharomyces cerevisiae.
33. The yeast cell of embodiment 31, wherein said yeast cell is Pichia pastoris.
34. A library comprising polynucleotides encoding a heterogeneous population of antibodies that may be displayed on the extracellular surface of the plasma membrane of a yeast cell.
35. The library of embodiment 34, wherein said heterogeneous population of antibodies comprise a library of heavy chain variable region sequences.
36. The library of embodiment 34, wherein said heterogeneous population of antibodies comprise a library of light chain variable region sequences.
37. The library of embodiment 34, wherein said heterogeneous population of antibodies comprise a library of single chain antibody sequences.
38. The library of embodiment 37, wherein said single chain antibody sequences further comprise an Fc region.
39. The library of embodiment 34, wherein said heterogeneous population of antibodies comprise a library of variant Fc regions.
40. The library of any one of embodiments 34-39, wherein said antibodies comprise an amino acid sequence that targets said antibodies to the cell surface, wherein said amino acid sequence is fused to the heavy chain or the light chain or the Fc region of said antibodies.
41. The library of embodiment 40, wherein said amino acid sequence is fused to the C-terminal end of the heavy chain or the light chain or the Fc region of the antibodies.
42. The library of embodiment 41, wherein said amino acid sequence comprises a transmembrane domain.
43. The library of embodiment 41, wherein said amino acid sequence comprises a GPI anchor domain.
44. The library of embodiment 42, wherein said transmembrane domain is derived from thrombomodulin, Axl2p, or Swp1p.
45. The library of embodiment 44, wherein said transmembrane domain comprises a polypeptide having an amino acid sequence corresponding to SEQ ID NO:2, 4, or 6.
46. A population of yeast cells comprising the library of any one of embodiments 34-45.
47. The population of yeast cells of embodiment 46, wherein said population of yeast cells comprise a genetic mutation rendering the cell wall sufficiently porous to make it permeable for a protein antigen or an antibody.
48. The population of yeast cells of embodiment 47, wherein said population of yeast cells comprise a genetic mutation in mnn9 or an orthologue of mnn9.
49. The population of yeast cells of embodiment 46, wherein said population of yeast cells are of a genus selected from the group consisting of: Saccharomyces, Pichia, Hansenula, Schizosaccharomyces, Kluyveromyces, Yarrowia, Debaryomyces and Candida.
50. The population of yeast cells of embodiment 49,wherein said population of yeast cells are selected from the group consisting of: Saccharomyces cerevisiae, Hansenula polymorpha, Kluyveromyces lactis, Pichia pastoris, Schizosaccharomyces pombe and Yarrowia lipolytica.
51. The population of yeast cells of embodiment 50, wherein said population of yeast cells are Saccharomyces cerevisiae.
52. The population of yeast cells of embodiment 50, wherein said population of yeast cells are Pichia pastoris.
53. A method of isolating an antibody having a desirable binding characteristic comprising:
   a) culturing a population of yeast cells comprising a library under conditions that allow display of an antibody on the extracellular surface of the plasma membrane;
   b) contacting said yeast cells with an enzyme that renders the cell wall sufficiently porous to make it permeable for an antibody ligand;
   c) contacting said yeast cells with an antibody ligand; and
   d) sorting said yeast cells based on the binding of said antibody ligand thereby isolating at least one cell expressing an antibody having the desired binding characteristic;
   wherein said library comprises polynucleotides encoding a heterogeneous population of antibodies that may be displayed on the extracellular surface of the plasma membrane of a yeast cell.
54. A method of isolating an antibody having a desirable binding characteristic comprising:
   a) culturing a population of yeast cells comprising a library under conditions that allow display of an antibody on the extracellular surface of the plasma membrane;
   b) contacting said yeast cells with an antibody ligand; and
   c) sorting said yeast cells based on the binding of said antibody ligand thereby isolating at least one cell expressing an antibody having the desired binding characteristic;
   wherein said library comprises polynucleotides encoding a heterogeneous population of antibodies that may be displayed on the extracellular surface of the plasma membrane of a yeast cell.
55. The method of any one of embodiments 53-54, wherein said ligand is labeled with a detectable agent.
56. The method of embodiment 55, wherein said detectable agent is selected from the group consisting of a fluorescent marker, biotin, streptavidin, and a peptide tag.
57. The method of any one of embodiments 53-56, further comprising the step of isolating a polynucleotide from the isolated yeast cell, wherein said polynucleotide encodes an antibody having a desirable binding characteristic.
58. The method of any one of embodiments 53-56, wherein said desirable binding characteristic is binding to a specific antigen.
59. The method of any one of embodiments 53-56, wherein said desirable binding characteristic is increased binding to a specific antigen.
60. The method of any one of embodiments 53-56, wherein said desirable binding characteristic is decreased binding to a specific antigen.
61. The method of any one of embodiments 53-56, wherein said desirable binding characteristic is binding to an effector molecule.
62. The method of any one of embodiments 53-56, wherein said desirable binding characteristic is reduced binding to an effector molecule.
63. The method of any one of embodiments 53-56, wherein said desirable binding characteristic is increased binding to an effector molecule.
64. The method of any one of embodiments 61 to 63, wherein said effector molecule is selected from the group consisting of C1q, FcγRI, FcγRII and FcγRIIIA.
65. The method of any one of embodiments 53-56, wherein said heterogeneous population of antibodies comprise a library of heavy chain variable region sequences.
66. The method of any one of embodiments 53-56, wherein said heterogeneous population of antibodies comprise a library of light chain variable region sequences.
67. The method of any one of embodiments 53-56, wherein said heterogeneous population of antibodies comprise a library of single chain antibody sequences.
68. The method of any one of embodiment 67, wherein said single chain antibody sequences further comprise an Fc region.
69. The method of any one of embodiments 53-56, wherein said heterogeneous population of antibodies comprise a library of variant Fc regions.
70. The method of any one of embodiments 53-69, wherein said antibodies comprise an amino acid sequence that targets said antibodies to the cell surface, wherein said amino acid sequence is fused to the heavy chain or the light chain or the Fc region of said antibodies.
71. The method of embodiment 70, wherein said amino acid sequence is fused to the C-terminal end of the heavy chain or the light chain or the Fc region of the antibodies.
72. The method of embodiment 71, wherein said amino acid sequence comprises a transmembrane domain.
73. The method of embodiment 71, wherein said amino acid sequence comprises a GPI anchor domain.
74. The method of embodiment 72, wherein said transmembrane domain is derived from thrombomodulin, Axl2p, or Swp1p.
75. The method of embodiment 74, wherein said transmembrane domain comprises a polypeptide having an amino acid sequence corresponding to SEQ ID NO:2, 4, or 6.
76. The method of embodiment 53, further comprising the steps of:
   i) cloning a polynucleotide encoding said antibody having the desired binding characteristic from the at least one cell isolated in step (d) and subcloning said polynucleotide into a vector adapted for expression in a eukaryotic cell; and
   ii) expressing said antibody in said eukaryotic cell, wherein said desired binding characteristic is confirmed by determining the properties of said antibody.
77. The method of embodiment 53, further comprising the steps of:
   i) cloning a polynucleotide encoding said antibody having the desired binding characteristic into a vector adapted for expression in a prokaryote; and
   ii) expressing said antibody in said prokaryote, wherein said desired binding characteristic is confirmed by determining the properties of said antibody.
78. The method of embodiment 76, wherein said eukaryotic cell is selected from the group consisting of mammalian, insect and yeast cells.
79. The method of embodiment 54, further comprising the steps of
   i) cloning a polynucleotide encoding said antibody having the desired binding characteristic into a vector adapted for expression in a eukaryotic cell; and
   ii) expressing said antibody in said eukaryotic cell, wherein said desired binding characteristic is confirmed by determining the properties of said antibody.
80. The method of embodiment 54, further comprising the steps of:
   i) cloning a polynucleotide encoding said antibody having the desired binding characteristic into a vector adapted for expression in a prokaryote; and
   ii) expressing said antibody in said prokaryote, wherein said desired binding characteristic is confirmed by determining the properties of said antibody.
81. The method of embodiment 79, wherein said eukaryotic cell is selected from the group consisting of mammalian, insect and yeast cells.
82. A method of expressing a library in a population of yeast cells, comprising:
   a) transforming a population of yeast cells with said library; and
   b) incubating said population of yeast cells under conditions sufficient for the production of antibodies that may be displayed on the extracellular surface of the plasma membrane of said population of yeast cells;
   wherein said library comprises polynucleotides encoding a heterogeneous population of antibodies that may be displayed on the extracellular surface of the plasma membrane of a yeast cell.
83. The method of embodiment 82, wherein said population of yeast cells comprise a genetic mutation rendering the cell wall sufficiently porous to make it permeable for a protein antigen or an antibody.
84. The method of embodiment 83, wherein said population of yeast cells comprise a genetic mutation in mnn9 or an orthologue of mnn9.
85. The method of any one of embodiments 82-84, wherein said population of yeast cells are of a genus selected from the group consisting of: Saccharomyces, Pichia, Hansenula, Schizosaccharomyces, Kluyveromyces, Yarrowia, Debaryomyces and Candida.
86. The method of embodiment 85,wherein said population of yeast cells are selected from the group consisting of: Saccharomyces cerevisiae, Hansenula polymorpha, Kluyveromyces lactis, Pichia pastoris, Schizosaccharomyces pombe and Yarrowia lipolytica.
87. The method of embodiment 86, wherein said population of yeast cells are Saccharomyces cerevisiae.
88. The method of embodiment 81, wherein said population of yeast cells are Pichia pastoris.
89. The method of embodiment 82, wherein said heterogeneous population of antibodies comprise a library of heavy chain variable region sequences.
90. The method of embodiment 82, wherein said heterogeneous population of antibodies comprise a library of light chain variable region sequences.
91. The method of embodiment 82, wherein said heterogeneous population of antibodies comprise a library of single chain antibody sequences.
92. The method of embodiment 86, wherein said single chain antibody sequences further comprise an Fc region.
93. The method of embodiment 82, wherein said heterogeneous population of antibodies comprise a library of variant Fc regions.
94. The method of any one of embodiments 82 or 89-93, wherein said antibodies comprise an amino acid sequence that targets said antibodies to the cell surface, wherein said amino acid sequence is fused to the heavy chain or the light chain or the Fc region of said antibodies.
95. The method of embodiment 94, wherein said amino acid sequence is fused to the C-terminal end of the heavy chain or the light chain or the Fc region of the antibodies.
96. The method of embodiment 95, wherein said amino acid sequence comprises a transmembrane domain.
97. The method of embodiment 95, wherein said amino acid sequence comprises a GPI anchor domain.
98. The method of embodiment 96, wherein said transmembrane domain is derived from thrombomodulin, Axl2p, or Swp1p.
99. The method of embodiment 96, wherein said transmembrane domain comprises a polypeptide having an amino acid sequence corresponding to SEQ ID NO:2, 4, or 6.
100. A kit comprising: a) the library of any one of embodiments 34-45.
101. The kit of embodiment 100, further comprising a yeast cell.
102. The kit of embodiment 101, wherein said yeast cell is of a genus selected from the group consisting of: Saccharomyces, Pichia, Hansenula, Schizosaccharomyces, Kluyveromyces, Yarrowia, Debaryomyces and Candida.
103. The kit of embodiment 102,wherein said yeast cell is selected from the group consisting of: Saccharomyces cerevisiae, Hansenula polymorpha, Kluyveromyces lactis, Pichia pastoris, Schizosaccharomyces pombe and Yarrowia lipolytica.
104. The kit of embodiment 103, wherein said yeast cell is Saccharomyces cerevisiae.
105. The kit of embodiment 103, wherein said yeast cell is Pichia pastoris.

### 6. EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### 6.1 Example 1

### Antibody Display on Yeast Plasma Membrane

The following sections describe the generation and characterization of antibody fusion polypeptides that are efficiently displayed on the extracellular surface of the yeast plasma membrane.

### 6.1.1 Construction of Light Chain Display Vector

A novel NheI restriction site was introduced into pYD1 (Invitrogen) at the 3′ end of the nucleotide sequences encoding the Aga2 signal peptide using the QuickChange kit (Stratagene) and the YD1 (SEQ ID NO:26) and YD2 (SEQ ID NO:27) primers. A polynucleotide encoding the full length light chain of the 12G3H11 anti-EphA2 antibody (SEQ ID NO:60) was PCR amplified using the YD3 (SEQ ID NO:28) and YD4 (SEQ ID NO:29) primers, digested with Nhe I restriction endonuclease, and ligated into the NheI PmeI digested modified pYD1 vector described above. The resulting light chain display vector is designated as pYD-LC; a schematic representation of pYD-LC is depicted in FIG. 2B. Primers used herein were designed to ensure that the polynucleotides encoding the Aga2 signal peptide and the light chain were operatively linked in the light chain display vector. Cloning procedures were performed following standard protocols. Identity of pYD-LC was confirmed using restriction digestion and dideoxynucleotide sequencing.

### 6.1.2 Construction of Heavy Chain Display Vectors

Schematic representation of the pYC2a-HC heavy chain display vector is shown in FIG. 2A. pYC2a-HC was built using the pYC2-E vector (Invitrogen) as a scaffold. First, a polynucleotide encoding the alpha factor signal peptide (SEQ ID NO:24) was amplified from total RNA isolated from the BJ5457 (ATCC) yeast strain using primers YD5 (SEQ ID NO:30) and YD6 (SEQ ID NO:31) following a standard RT-PCR protocol. The amplified polynucleotide was digested with HindIII restriction endonuclease and ligated into a PvuII HindIII cleaved pYC2-E vector to generate pYCa-E. Second, a polynucleotide encoding a fusion polypeptide consisting of a full length heavy chain of the 12G3H11 anti-EphA2 antibody (SEQ ID NO:59) and the transmembrane domain of human thrombomodulin (SEQ ID NO:1) was generated via overlap PCR. The template for the reaction is an expression construct comprising the heavy chain gene of the 12G3H11 anti-EphA2 antibody with an engineered Afl II restriction site at its 3′ end. The reaction mix comprised a single forward primer, YD7 (SEQ ID NO:32), and a set of two reverse primers, YD8 (SEQ ID NO:33) and YD9 (SEQ ID NO:34). The overlap PCR reaction was performed following a standard protocol. The amplified polynucleotide is digested with XhoI restriction endonuclease and ligated into an XhoI PmeI cleaved pYCa-E vector to generate pYCa-HC-ThrmTM. The pYCa-HC-ThrmTM vector may be used to target a heavy chain for display on the surface of the plasma membrane. Third, a polynucleotide encoding the Aga1 gene GPI anchor domain was amplified from total RNA isolated from the BJ5457 yeast strain using primers YD10 (SEQ ID NO:35) and YD11 (SEQ ID NO:36) following a standard RT-PCR protocol. The PCR product was digested with AflII and HindIII restriction endonucleases and cloned into a similarly digested pYCa-HC-ThrmTM vector to generate pYC2a-HC-AgaGPI. The pYC2a-HC-AgaGPI vector may be used to target a heavy chain for display on the cell wall. Fourth, a polynucleotide encoding the Axl2p transmembrane domain was amplified from total RNA isolated from the BJ5457 yeast strain using primers YD14 (SEQ ID NO:39) and YD15 (SEQ ID NO:40) following a standard RT-PCR protocol. The PCR product was digested with AflII and HindIII restriction endonucleases and cloned into a similarly digested pYCa-HC-ThrmTM vector to generate pYC2a-HC-Axl2™. The pYC2a-HC-Axl2TM vector may be used to target a heavy chain for display on the surface of the plasma membrane. Fifth, a polynucleotide encoding the Swp1p transmembrane domain was amplified from total RNA isolated from the BJ5457 yeast strain using primers YD12 (SEQ ID NO:37) and YD13 (SEQ ID NO:38) following a standard RT-PCR protocol. The PCR product was digested with AflII and HindIII restriction endonucleases and cloned into a similarly digested pYCa-HC-ThrmTM vector to generate pYC2a-HC-Swp1TM. The pYC2a-HC-Swp1TM vector may be used to target a heavy chain for display on the surface of the plasma membrane. Cloning procedures were performed following standard protocols. Identity of the various vectors was confirmed using restriction digestion and dideoxynucleotide sequencing.

TABLE 2
List of primers used to generate the antibody display vectors.

| | | |
|---|---|---|
| YD1 | CAATATTTTCTGTTATTGCTAGCGTTTTAGCACAGGAACTGAC | SEQ ID NO: 26 |
| YD2 | GTCAGTTCCTGTGCTAAAACGCTAGCAATAACAGAAAATATTG | SEQ ID NO: 27 |
| YD3 | GTTATTGCTAGCGTTTTAGCAGACATCCAGATGACCCAGTCT | SEQ ID NO: 28 |
| YD4 | TTGCGGCCGCTATACTAGTGACATCGATTCACTAACACTCT | SEQ ID NO: 29 |
| YD5 | AACTAGTAAAAGAATGAGATTTCCTTCAATTTTTACTGC | SEQ ID NO: 30 |
| YD6 | GGTAATAAGCTTGAATTCAGCTTCAGCCTCTCTTTTCTCGAGAGA | SEQ ID NO: 31 |
| YD7 | GTATCTCTCGAGAAAAGACAAATGCAGCTGGTGCAGTCT | SEQ ID NO: 32 |
| YD8 | AGCGCCACCACCAGGCACAGGCTCGCGATGGAGATGCCTATG | SEQ ID NO: 33 |
| | AGCAAGCCTCCTTTACCCGGAGACAGGCTTAAG | |
| YD9 | TAGCGGCCGCTCACTGCTTCTTGCGCAGGTGGCAGAGGAGCGC | SEQ ID NO: 34 |
| | CAAAAGCGCCACCACCAGGCACAGGCT | |
| YD10 | AAGAGCTTAAGCCTGTCTCCGGGTAAAGGCGGTGGCGGAAGC | SEQ ID NO: 35 |
| | GCCAAAAGCTCTTTTATC | |
| YD11 | AGCGGGTTTGCGGCCGCTCATTAGAATAGCAGGTACGACAAAAG | SEQ ID NO: 36 |
| YD12 | GAAGAGCTTAAGCCTGTCTCCGGGTAAACAACTCAACCTGAACTT | SEQ ID NO: 37 |
| | CGATGTAG | |
| YD13 | CGGGTTTGCGGCCGCTCATTAAGTGACACCGGTCGGGATGTTATTG | SEQ ID NO: 38 |
| YD14 | GAAGAGCTTAAGCCTGTCTCCGGGTAAAACAAGTTCTTACACATCT | SEQ ID NO: 39 |
| | TCTAC | |
| YD15 | CGGGTTTGCGGCCGCTCATTAGGAAGCATCATCATCAAAGGGGTT | SEQ ID NO: 40 |
| | G | |
| YD16 | GGTATCTCTCGAGAAAAGAGAGGTGCAGCTGGTGGAGTCTGGGGG | SEQ ID NO: 41 |
| YD17 | AGACAGGCTTAAGCAAGATTTGGGCTCAACTCTCTTGTCCACCTT | SEQ ID NO: 42 |
| YD18 | GTTATTGCTAGCGTTTTAGCACAGTCTGTGCTGACGCAGCCGCC | SEQ ID NO: 43 |
| YD19 | TCCTTTACCCGGAGACAGGCTTAAGGAACATTCTGTAGGGGCCACT | SEQ ID NO: 44 |
| | GT | |
| YD20 | TTAAGCCTGTCTCCGGGTAAAGGA | SEQ ID NO: 45 |
| YD21 | GTCACGCTTACATTCACGC | SEQ ID NO: 46 |
| YD22 | GGGTTTGCGGCCGCTCATTAACAAGATTTGGGCTCAACTCTCTT | SEQ ID NO: 47 |
| YD23 | TCTCTCGAGAAAAGAGACTACAAAGATGACGATGACAAAGAGGTG | SEQ ID NO: 48 |
| | CAGCTGGTGGAGTCT | |
| YD24 | GGAGCCGCCGCCGCCAGAACCACCACCACCTGAGGAGACGGTGAC | SEQ ID NO: 49 |
| | CATGGT | |
| YD25 | TCTGGCGGCGGCGGCTCCGGTGGTGGTGGTTCTGCCATCCAGTTGA | SEQ ID NO: 50 |
| | CTCAGTCT | |
| YD26 | GACAGGCTTAAGCCTTTGATCTCCAGCTTGGTCCCT | SEQ ID NO: 51 |
| TD27 | TCTCTCGAGAAAAGAGACTACAAAGATGACGATGACAAACAGGTG | SEQ ID NO: 52 |
| | CAGCTGCAGGAGTC | |
| YD28 | GGAGCCGCCGCCGCCAGAACCACCACCACCTGAGGAGACGGTGAC | SEQ ID NO: 53 |
| | CAGGGT | |
| YD29 | TCTGGCGGCGGCGGCTCCGGTGGTGGTGGTTCTCAGTCTGTGTTGA | SEQ ID NO: 54 |
| | CGCAGCCG | |
| YD30 | GACAGGCTTAAGCCTAGGACGGTCAGCTTGGTCCCT | SEQ ID NO: 55 |
| YD31 | TTTGGGGTCGACTTTGATCTCCAGCTTGGTCCCT | SEQ ID NO: 56 |
| YD32 | GTCGACCCCAAATCTAGTGACAAAACTCAC | SEQ ID NO: 57 |
| YD33 | GGCCCTTGGTCGACGCTGAGGAGACGGTGACCATGGTGCCC | SEQ ID NO; 58 |

### 6.1.3 Antibody Display on the Yeast Plasma Membrane

The strain of S. cerevisiae cells used in the experiments described herein is BJ5457 (ATCC) with a genotype of MATalpha ura3-52 trp1 lys2-801 leu2-delta1 his3-delta200 pep4::HIS3 prb1-delta1.6R can1 GAL unless otherwise stated.

The pYD-LC-10C12 vector comprising a polynucleotide encoding the light chain of the 10C12 anti-EphA4 antibody was generated by: 1) PCR amplifying a polynucleotide encoding the light chain variable region of 10C12; 2) cloning the NheI/BsiWI digested isolated PCR fragment into a similarly cut pYD-LC vector. The pYCa-HC-AgaGPI-10C12, pYCa-HC-ThrmTM-10C12, pYCa-HC-Axl2TM-10C12, and pYCa-HC-Swp1TM-10C12 vectors, each of which comprises a polynucleotide encoding the heavy chain of the 10C12 anti-EphA4 antibody, were generated by: 1) PCR amplifying a polynucleotide encoding the heavy chain variable region of 10C12; 2) cloning the XhoI/SalI digested isolated PCR fragment into a similarly cut pYCa-HC-AgaGPI, pYCa-HC-ThrmTM, pYCa-HC-Axl2TM, and pYCa-HC-Swp1TM, vector, respectively.

S. cerevisiae cells are co-transformed with a light chain and heavy chain expression vector comprising the 10C12 anti-EphA4 antibody using a S.c. EasyComp™ Transformation Kit (Invitrogen). The following vector pairs were tested: pYD-LC-10C12 with pYCa-HC-AgaGPI-10C12, pYD-LC-10C12 with pYCa-HC-ThrmTM-10C12, pYD-LC-10C12 with pYCa-HC-Axl2TM-10C12, and pYD-LC-10C12 with pYCa-HC-Swp1TM-10C12. Transformed cells were plated onto Trp-Ura-SDCAA plates and incubated at 30° C. for 36 hours. 4 ml SD-CAA 2% dextrose containing yeast minimal growth medium is inoculated with a single colony and grown overnight on an orbital shaker (30° C., 300 rpm). Cells are harvested by centrifugation, resuspended in 20 ml of SG-CAA galactose containing yeast minimal induction medium and incubated at 20° C. overnight on an orbital shaker (300 rpm) to allow for antibody expression. Induced cells are harvested and digested with lyticase (Sigma, 100 U/ml stock, final concentration is 1 U/10⁶ cells) in the presence of 1 M sorbitol, 0.1% beta-mercaptoethanol, and 0.1 mM EDTA at 30° C. for 30 minutes to generate spheroplasts. Spheroplasts are subsequently incubated with FITC conjugated anti-human IgG(H+L) antibody (PIERCE) in the presence of 1 M sorbitol for 30 minutes followed by analysis on a flow cytometer. Spheroplasts prepared from uninduced control yeast cells are used as negative control.

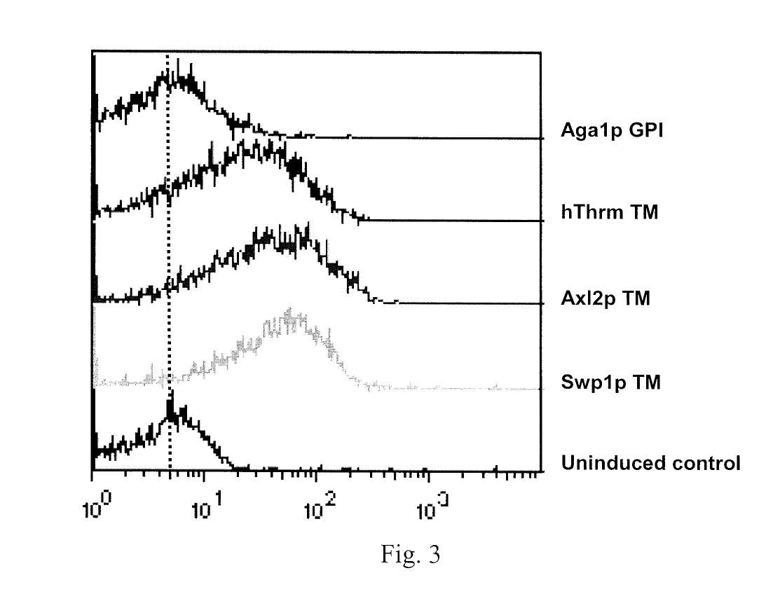

Spheroplasts prepared from galactose induced yeast cells comprising the pYCa-HC-ThrmTM-10C12, pYCa-HC-Axl2TM-10C12, or pYCa-HC-Swp1TM-10C12 heavy chain display vectors show significant positive staining compared to the negative control samples. Spheroplasts prepared from galactose induced yeast cells comprising the pYCa-HC-AgaGPI-10C12 heavy chain display vector are stained at a level comparable to that of the negative control sample, presumably because the lyticase digestion removed the cell wall displayed antibody along with the cell wall itself. The observed results indicate that a yeast spheroplast plasma membrane displayed antibody is accessible to large molecular weight reagents. A representative example of the flow cytometry results is shown in FIG. 3.

### 6.1.4 Optimization of Spheroplast Formation

S. cerevisiae cells comprising either the pYD-LC-10C12/pYCa-HC-AgaGPI-10C12 or the pYD-LC-10C12/pYCa-HC-ThrmTM-10C12 antibody display vector pairs are subjected to galactose induction as described in 6.1.3. Cells are harvested and subjected to lyticase treatment using 100 U/ml lyticase in the presence or absence of 0.5 M sorbitol. Lyticase digestion is performed at 30° C. for 30 minutes. The effect of pH changes on spheroplast generation is assayed by performing lyticase treatment in buffers with different pH levels (e.g., pH 7.4, pH 7.6, pH 7.9, pH 9.0, pH9.7). Spheroplasts are incubated with FITC conjugated anti-human IgG(H+L) antibody in a PBS buffer (pH 7.2) containing 0.5% BSA and 1 mM EDTA at room temperature for 30 minutes. Stained samples are analyzed on a flow cytometer. “No lyticase” control samples are prepared by incubating the galactose induced yeast cells in a 0.5 M sorbitol containing lyticase treatment buffer (pH 9.6) with no lyticase enzyme for 30 minutes at 30° C. Staining and analysis of control samples is done using the same condition as used for the experimental samples.

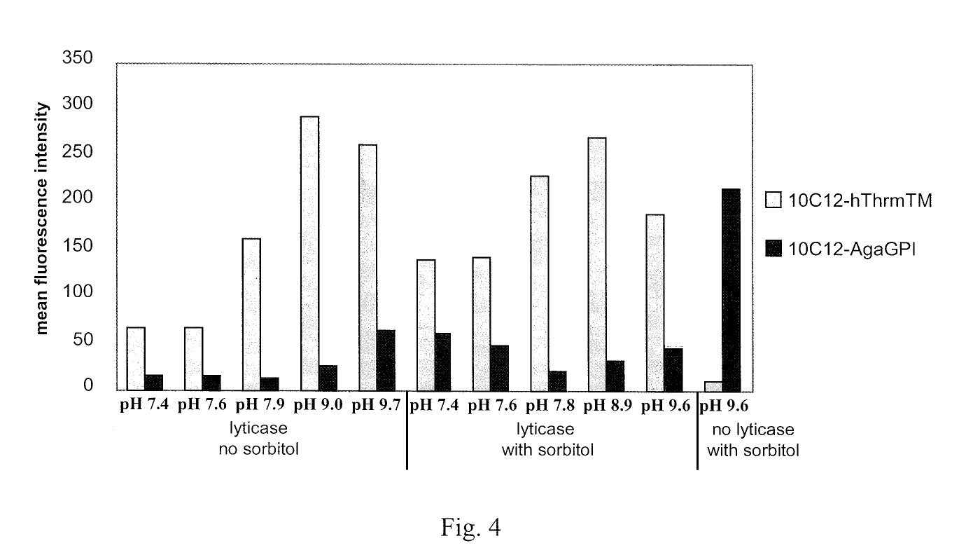

As expected, the staining intensity of spheroplasts comprising the pYD-LC-10C12/pYCa-HC-ThrmTM-10C12 antibody display vector pair is higher than that of the intact cells comprising the same vector pair; indicating that enzymatic removal of the cell wall renders the plasma surface displayed antibody accessible to the FITC labeled binding reagent. On the other hand, spheroplasts comprising the pYD-LC-10C12/pYCa-HC-AgaGPI-10C12 antibody display vector pair display significantly lower staining intensity than that of the intact cells comprising the same vector pair; indicating that the enzymatic removal of the cell wall also removes the cell wall displayed antibody. Staining intensity of spheroplasts comprising the pYD-LC-10C12/pYCa-HC-ThrmTM-10C12 antibody display vector pair varies with the pH of the lyticase treatment buffer. Highest staining intensity is seen with samples that were digested in a buffer with a pH of or above 7.9. The presence or absence of 0.5 M sorbitol in the lyticase digestion buffer does not seem to significantly affect the final staining intensity of spheroplasts under the conditions used. A chart summarizing the results of a representative experiment is shown in FIG. 4.

### 6.2 Example 2

### Antigen Binding Properties of Yeast Plasma Membrane Displayed Antibodies

The following sections describe experiments demonstrating that antibodies displayed on the extracellular surface of the yeast plasma membrane can efficiently bind their target antigen

### 6.2.1 A Yeast Plasma Membrane Displayed 10C12 Antibody Efficiently Binds its Target Antigen

S. cerevisiae cells comprising the pYD-LC-10C12/pYCa-HC-ThrmTM-10C12 antibody display vector pair are subjected to galactose induction as described in 5.1.3. Following induction, cells are harvested and treated with 100 U/ml lyticase in a 0.5 M sorbitol containing buffer pH 9.6 for 30 minutes at 30° C. Spheroplasts are incubated with biotinylated EphA4-Fc fusion protein (50n/ml) in FACS buffer (PBS (pH 7.2), 0.5% BSA, 1 mM EDTA) for 30 minutes at room temperature. Spheroplasts are subsequently washed with FACS buffer, followed by incubation with 1:200 fold diluted PE conjugated streptavidin (Pierce) on ice for 30 minutes. Stained spheroplast are analyzed using a flow cytometer.

Two control samples are prepared and analyzed in parallel with the experimental sample. The first control sample is spheroplasts prepared from uninduced yeast cells comprising the pYD-LC-10C12/pYCa-HC-ThrmTM-10C12 antibody display vector pair; spheroplast preparation and staining is identical to experimental sample. The second control sample is prepared from galactose induced yeast cells comprising the pYD-LC-10C12/pYCa-HC-AgaGPI-10C12 antibody display vector pair; the second control sample comprises intact yeast cells immunostained following the same protocol as the one used for the experimental sample.

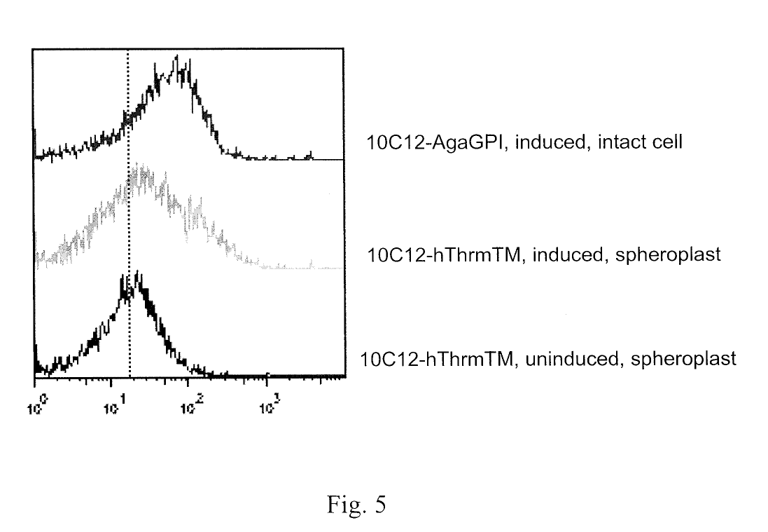

Spheroplasts prepared from galactose induced yeast cells comprising the pYD-LC-10C12/pYCa-HC-ThrmTM-10C12 antibody display vector pair show a significantly higher staining intensity than that of the spheroplasts prepared from uninduced yeast cells; indicating that enzymatic removal of the cell wall renders the plasma surface displayed antibody accessible to the biotinylated antigen and PE conjugated streptavidin. As expected, intact galactose induced yeast cells comprising the pYD-LC-10C12/pYCa-HC-AgaGPI-10C12 antibody display vector pair also displayed high staining intensity; indicating that the cell wall displayed 10C12 antibody is capable of antigen binding. The observed high staining intensity of galactose induced spheroplast comprising the pYD-LC-10C12/pYCa-HC-ThrmTM-10C12 antibody display vector pair indicates that the yeast plasma membrane displayed 10C12 anti-EphA4 antibody is capable of antigen binding. A representative example of the flow cytometry results is shown in FIG. 5.

### 6.2.2 A Yeast Plasma Membrane Displayed 3F2 Fab Efficiently Binds its Target Antigen

The p3F2Fd-ThrmTM antibody display vector having a polynucleotide encoding a fusion polypeptide comprising (i) the Fd heavy chain fragment of the 3F2 anti-EphA2 antibody and (ii) the transmembrane domain of human thrombomodulin is constructed as follows. The Fd region of 3F2, a humanized anti-EphA2 antibody, is PCR amplified using primers YD16 and YD17, digested by Xho I and Afl II restriction endonucleases, and ligated into a similarly digested pYCa-HC-ThrmTM vector to generate p3F2Fd-ThrmTM.

The p3F2LC-ThrmTM antibody display vector having a polynucleotide encoding a fusion polypeptide comprising the light chain of the 3F2 anti-EphA2 antibody and the transmembrane domain of human thrombomodulin (3F2LC-ThrmTM) is constructed as follows. A first polynucleotide encoding the 3F2 light chain polypeptide is generated by PCR amplification using primers YD18 and YD19. A second polypeptide encoding the transmembrane domain of human thrombomodulin is generated by PCR amplification from the pYCa-HC-ThrmTM template using primers YD20 and 21. A third polynucleotide encoding the 3F2LC-ThrmTM fusion polypeptide is generated by fusing the first and second polynucleotide via overlap-PCR using primers the YD18 and YD21 primers. The third polynucleotide is digested with NheI and NotI, and ligated into a similarly digested pYD-LC vector to generate p3F2LC-ThrmTM.

The p3F2Fd antibody display vector having a polynucleotide encoding the Fd heavy chain fragment of the 3F2 anti-EphA2 antibody is constructed as follows. The Fd region of the 3F2 heavy chain is PCR amplified using primers YD16 and YD22, digested by Xho I and Not I, and cloned into similarly digested pYCa-HC-ThrmTM to generate vector p3F2Fd.

The pYD-LC-3F2 vector comprising a polynucleotide encoding the light chain of the 3F2 anti-EphA2 antibody was generated by: 1) PCR amplifying a polynucleotide encoding the light chain variable region of 3F2; 2) cloning the NheI/BsiWI digested isolated PCR fragment into a similarly cut pYD-LC vector.

S. cerevisiae cells comprising the pYD-LC-3F2/p3F2Fd-ThrmTM or p3F2LC-ThrmTM/p3F2Fd antibody display vector pair are subjected to galactose induction as described in 5.1.3. Following induction, cells are harvested and treated with 100 U/ml lyticase in a 0.5 M sorbitol containing buffer (pH 9.4) for 30 minutes at 30° C. Spheroplasts are divided into two batches. The first batch is incubated with 18 microgram/ml biotinylated EphA2-Fc fusion protein (R&D Systems) in FACS buffer (PBS (pH 7.2), 0.5% BSA, 1 mM EDTA) for 30 minutes at room temperature, then washed with FACS buffer, and subsequently stained with 1:2000 fold diluted PE conjugated streptavidin (Pierce) on ice for 30 minutes. The second batch of spheroplasts is stained with FITC conjugated anti-human IgG(H+L) antibody (PIERCE) in a PBS buffer (pH 7.2) containing 0.5% BSA and 1 mM EDTA at room temperature for 30 minutes. Stained spheroplast are analyzed using a flow cytometer. A control sample of spheroplasts prepared from uninduced yeast cells comprising the pYD-LC-3F2/p3F2Fd-ThrmTM antibody display vector pair is included in the experiment.

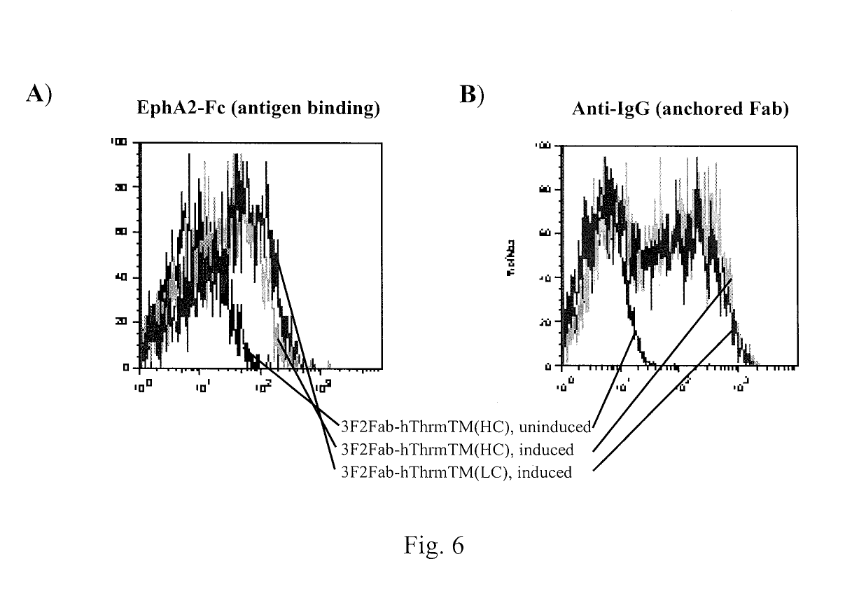

Spheroplasts prepared from galactose induced yeast cells comprising the pYD-LC-3F2/p3F2Fd-ThrmTM or p3F2LC-ThrmTM/p3F2Fd antibody display vector pair show identical high staining intensity with FITC conjugated anti-human IgG(H+L) antibody (PIERCE); indicating that an antibody fragment is efficiently displayed on the surface of the plasma membrane when either the heavy chain fragment or the light chain comprises a transmembrane domain. The staining intensity of galactose induced spheroplasts is significantly higher than that of the control uninduced spheroplasts; indicating that galactose induced expression of the plasma membrane displayed antibody fragment is a necessary condition for getting a positive staining signal. Spheroplasts prepared from galactose induced yeast cells comprising the pYD-LC-3F2/p3F2Fd-ThrmTM or p3F2LC-ThrmTM/p3F2Fd antibody display vector pair show identical high staining intensity with biotinylated EphA2-Fc/PE conjugated streptavidin; indicating that a plasma membrane displayed antibody fragment efficiently binds its cognate antigen regardless of whether the heavy chain fragment or the light chain comprises the transmembrane domain. The staining intensity of galactose induced spheroplasts is significantly higher than that of the control uninduced spheroplasts; indicating that enzymatic removal of the cell wall renders the plasma surface displayed antibody fragment accessible to the FITC labeled binding reagent. The observed results indicate that a yeast plasma membrane displayed Fab fragment anchored by either the heavy chain Fd fragment or the light chain can efficiently bind its target antigen. A representative example of the flow cytometry results is shown in FIG. 6.

6.2.3 A Yeast Plasma Membrane Displayed 3F2 scFv Efficiently Binds its Target Antigen

The pFLAG3F2scFv-ThrmTM antibody display vector having a polynucleotide encoding a fusion polypeptide comprising (i) the FLAG tag, (ii) the 3F2 anti-EphA2 scFv, and (iii) the transmembrane domain of human thrombomodulin is constructed as follows. Polynucleotides encoding the FLAG tagged VH and the untagged VL regions of the 3F2 anti-EphA2 antibody are PCR amplified using primer pairs YD23/YD24 and YD25/YD26, respectively. These two polynucleotides are fused into a single polynucleotide encoding the FLAG tagged 3F2 scFv by over-lapping PCR using primers YD23 and YD26. The assembled PCR product is digested by Xho I/Afl II and cloned into a similarly digested pYCa-HC-ThrmTM vector to generate pFLAG3F2scFv-ThrmTM.

The pFLAG10C12scFv-ThrmTM antibody display vector having a polynucleotide encoding a fusion polypeptide comprising (i) the FLAG tag, (ii) the 10C12 anti-EphA4 scFv, and (iii) the transmembrane domain of human thrombomodulin is constructed as follows. Polynucleotides encoding the FLAG tagged VH and untagged VL regions of the 12C12 anti-EphA4 antibody are PCR amplified using primer pairs YD27/YD28 and YD29/YD30, respectively. These two polynucleotides are fused into a single polynucleotide encoding the FLAG tagged 10C12 scFv by over-lapping PCR using primers YD27 and YD30. The assembled PCR product is digested by Xho I/Afl II and cloned into a similarly digested pYCa-HC-ThrmTM vector to generate pFLAG10C12scFv-ThrmTM.

S. cerevisiae cells comprising the pFLAG3F2scFv-ThrmTM antibody display vector are subjected to galactose induction as described in 5.1.3. Following induction, cells are harvested and treated with 100 U/ml lyticase in a 0.5 M sorbitol containing buffer (pH 9.4) for 30 minutes at 30° C. Spheroplasts are stained with 10 microgram/ml FITC conjugated anti-FLAG antibody in a PBS buffer (pH 7.2) containing 0.5% BSA and 1 mM EDTA at room temperature for 30 minutes. Stained spheroplast are analyzed using a flow cytometer. A negative control sample of spheroplasts prepared from uninduced yeast cells is also analyzed.

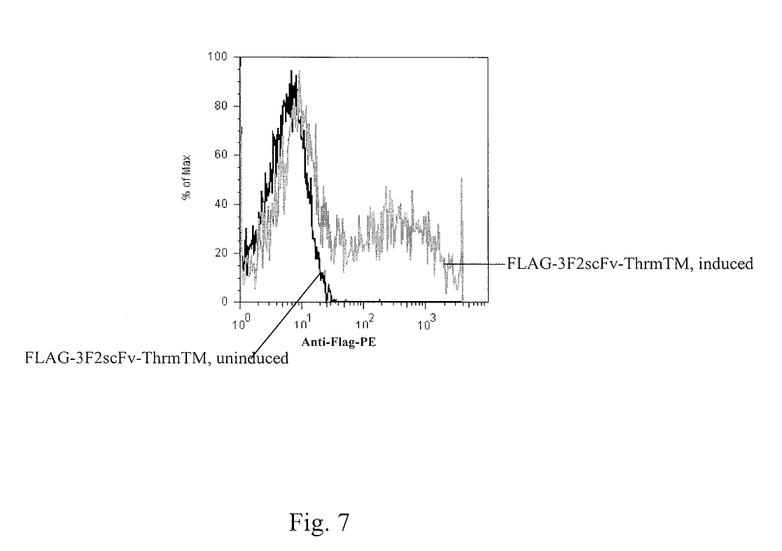

Spheroplasts prepared from galactose induced yeast cells comprising the pFLAG3F2scFv-ThrmTM antibody display vector show significantly higher staining than that of the spheroplasts prepared from uninduced yeast cells. The observed results indicate that a yeast spheroplast plasma membrane displayed scFv is accessible for interaction with large molecular weight reagents (e.g., antibodies). A representative example of the flow cytometry results is shown in FIG. 7.

6.2.4 A Yeast Plasma Membrane Displayed 3F2 scFv-Fc Antibody Efficiently Binds its Antigen

The p3F2scFv-Fc-ThrmTM antibody display vector having a polynucleotide encoding a fusion polypeptide comprising (i) the 3F2 anti-EphA2 scFv, (ii) a heavy chain Fc region, and (iii) the transmembrane domain of human thrombomodulin is constructed as follows. A polynucleotide encoding the 3F2scFv fragment is PCR amplified from the pFLAG3F2scFv-ThrmTM template using the YD16 and YD31 primers. A polynucleotide encoding the IgG1 Fc region fused to the transmembrane domain of human thrombomodulin is PCR amplified from the pYCa-HC-ThrmTM template using the YD32 and YD21 primers. These two PCR products are assembled into a single polynucleotide encoding the 3F2scFv-Fc-TM fusion polypeptide by overlap-PCR using primer pair YD16/YD21. The final PCR product is digested by Xho I/Not I and ligated into a similarly digested pYCa-HC-ThrmTM vector to generate p3F2scFv-Fc-Thrm™.

The pYCa-HC-ThrTM-3F2 antibody display vector having a polynucleotide encoding a fusion polypeptide comprising (i) the heavy chain of the 3F2 anti-EphA2 antibody, and (ii) the transmembrane domain of human thrombomodulin is constructed as follows. A polynucleotide encoding the VH domain of 3F2 is PCR amplified using primers YD16 and YD33, digested by Xho I/Sal I and cloned into a similarly digested pYCa-HC-ThrmTM vector to generate pYCa-HC-ThrmTM-3F2.

S. cerevisiae strain LB 3003-4B (MATalpha mnn9 ur3 leu2 his4) cells (ATCC), hereinafter denoted as mnn9 cells, comprising either the p3F2scFv-Fc-TM or pYCa-HC-ThrmTM-3F2 antibody display vector are grown in 3 ml SD-CAA (-Ura, -Trp, -Ade) medium overnight at 30° C. on an orbital shaker (300 rpm). Cells are transferred into 30 ml galactose containing SG-CAA (-Ura, -Trp, -Ade) medium and incubated at 20° C. on an orbital shaker (300 rpm) for 20 hrs to induce the expression of the plasma membrane displayed 3F2scFv-Fc-TM antibody. Following induction, the cell density is determined by measuring the absorbance of the culture at 600 nm wavelength. 0.1 absorbance unit, corresponding to approximately 1×10⁶ cells, of the culture is harvested by 2 minutes centrifugation at 14,000 rpm and washed with PBS/1% BSA buffer. A control sample of mnn9 cells having no antibody display vector is also subjected to the above described galactose induction. Induced cells are stained either with FITC conjugated anti-human Fc antibody or biotinylated EphA2/PE conjugated streptavidin as follows.

FITC conjugated anti-human Fc antibody staining: Cells are resuspended in 50 μl of 1:150 fold diluted FITC conjugated anti-human Fc antibody (PIERCE) in PBS/1% BSA and incubated on ice for 30-60 minutes. Cells are washed with PBS/1% BSA, resuspended in 350 μl of PBS/1% BSA and analyzed on a flow cytometer. Positive staining signal indicates that the scFv-Fc antibody is displayed on the surface of the plasma membrane, and is accessible to staining

Biotinylated EphA2/PE conjugated streptavidin staining: Cells are resuspended in 50 μl of 18 mg/ml biotinylated EphA2 (R&D Systems)/PBS/1% BSA and incubated on ice for 60 minutes. Cells are washed with PBS/1% BSA, resuspended in 50 μl of 1:200 fold diluted PE conjugated streptavidin (PIERCE)/PBS/1% BSA and incubated on ice for 30 minutes. Cells are washed with PBS/1% BSA, resuspended in 350 μl of PBS/1% BSA and analyzed on a flow cytometer. Positive staining signal indicates that the plasma membrane displayed scFv-Fc antibody is accessible to the antigen, and is capable of antigen binding.

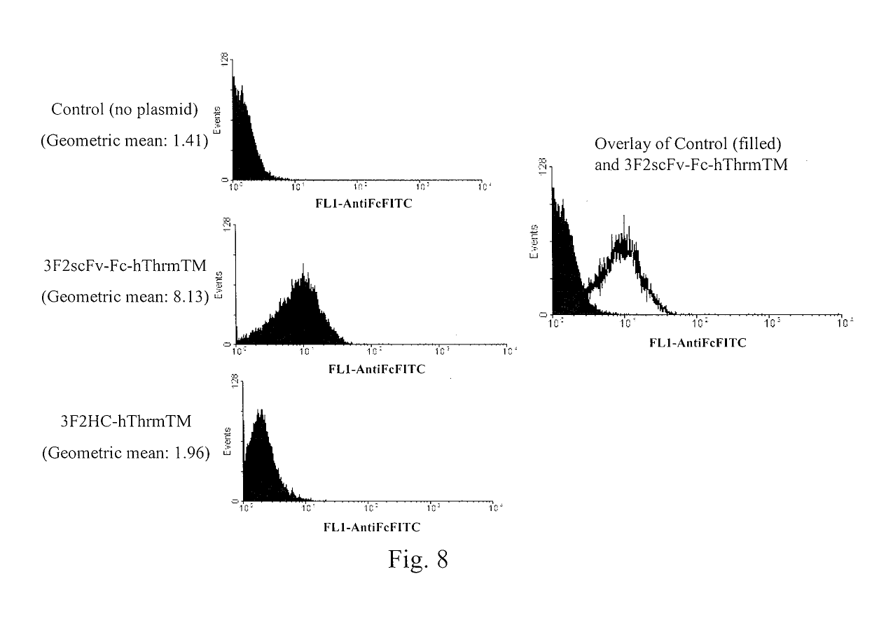

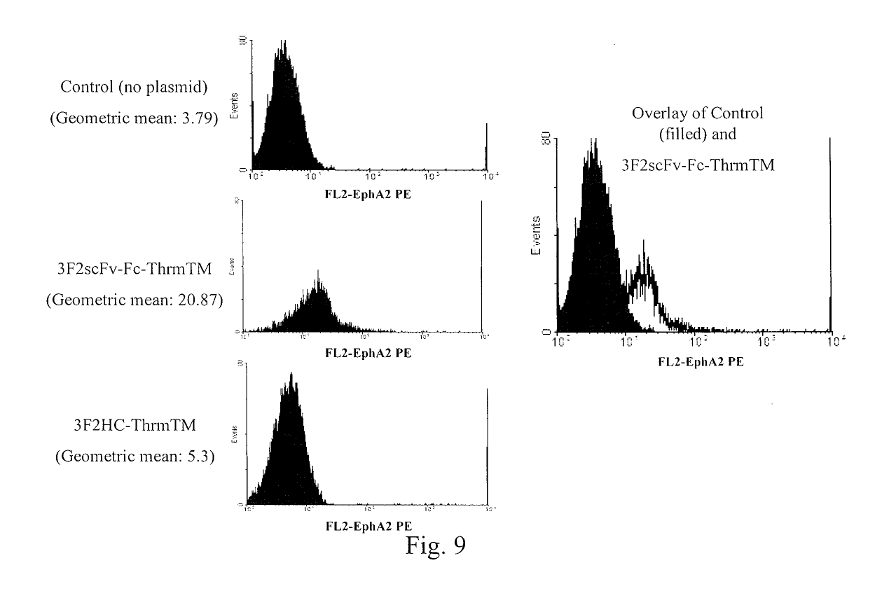

The staining intensity patterns observed with FITC conjugated anti-human Fc antibody and biotinylated EphA2/PE conjugated streptavidin are identical. In both cases the staining intensity of galactose induced mnn9 cells comprising the p3F2scFv-Fc-ThrmTM antibody display vector is significantly higher than that of the control mnn9 cells comprising no antibody display vector. The staining intensity of mnn9 cells comprising the pYCa-HC-ThrmTM-3F2 antibody display vector is the same as that of the control mnn9 cells comprising no antibody display vector; indicating that the 3F2 heavy chain fusion polypeptide comprising a transmembrane domain is not efficiently displayed on the surface of the plasma membrane in the absence of the 3F2 light chain. The observed data demonstrates that a) 3F2scFv-Fc-ThrmTM antibody is efficiently displayed on the yeast plasma membrane, b) in mnn9 cells a plasma membrane displayed antibody is accessible for interaction high molecular weight reagents (e.g., anti-human Fc antibody, biotinylated EphA2 protein), c) a yeast plasma membrane displayed 3F2scFv-Fc-ThrmTM antibody is capable to efficiently bind its cognate antigen. A representative example of the flow cytometry results is shown in FIGS. 8 and 9.

### 6.3 Example 3

### . Method for Screening an Artificial Yeast Plasma Membrane Displayed Full Length Antibody Library

The following section describes a non-limiting example of method for screening and isolating cells displaying a full antibody on their plasma membrane from a population of yeast cells comprising an artificial library. The population of yeast cells comprising an artificial library is generated by mixing yeast cells comprising either the pYD-LC-10C12/pYCa-HC-ThrmTM-10C12 (full length antibody) or the pYD-LC-3F2/p3F2Fd-ThrmTM (antibody fragment) antibody display vector pair at a ratio of 1 to 10⁴.

First round of selection: Yeast cells comprising the pYD-LC-10C12/pYCa-HC-ThrmTM-10C12 or the pYD-LC-3F2/p3F2Fd-ThrmTM antibody display vector pair are grown in CAA/2% glucose medium overnight at about 30° C. on an orbital shaker (about 300 rpm). Cells are harvested, resuspended in CAA-RGD (2% raffinose, 2% galactose, 0.1% glucose) medium and incubated overnight at about 20° C. on an orbital shaker (about 300 rpm). Cell density is determined by measuring the absorbance of the cultures at 600 nm wavelength. OD 1 corresponds approximately to a cell density of 1×10⁷ cells/ml. 10⁴ cells comprising the pYD-LC-10C12/pYCa-HC-ThrmTM-10C12 antibody display vector pair are mixed with 10⁸ cells comprising the pYD-LC-3F2/p3F2Fd-TM antibody display vector pair to create an artificial library. The cells are harvested by 2 minutes centrifugation at about 14,000 rpm, resuspended in of sodium carbonate buffer (pH 9.6), digested with 100 U/ml lyticase for 30 minutes at 30° C., and are twice washed with PBS (pH 9.6). Spheroplasts are incubated in of 1:150 fold diluted immunoPure FITC conjugated rabbit anti-human IgG(Fc) antibody (e.g., PIERCE) in PBS (pH 7.4)/1% BSA for about 30 minutes at room temperature. Stained spheroplasts are twice washed with PBS/1% BSA and sorted on a FACS machine. Positively stained cells are grown in CAA/2% glucose medium with Carbenicillin (50 μg/ml)/Tetracycline (5 μg/ml) (e.g., Sigma) for 2 days at 30° C. A small amount (˜1 μl) of the liquid culture is plated onto a 100 mm CAA-glucose plate and incubated at 30° C. for 3 days; the remainder of the liquid culture is subjected to a second round of selection.

Second round of selection: Cells expanded from the first round of selection are harvested and resuspended in CAA-RGD (2% raffinose, 2% galactose, 0.1% glucose) medium and incubated overnight at 20° C. on an orbital shaker (300 rpm). Cell density is determined by measuring the absorbance of the cultures at 600 nm wavelength. OD 1 corresponds approximately to a cell density of 1×10⁷ cells/ml. 5×10⁷ cells are harvested by 2 minutes centrifugation at 14,000 rpm, resuspended in of sodium carbonate (pH 9.6) buffer, digested with 100 U/ml lyticase for 30 minutes at 30° C., and are twice washed with PBS (pH 9.6). Spheroplasts are incubated in 1:150 fold diluted immunoPure FITC conjugated rabbit anti-human IgG(Fc) antibody (e.g., PIERCE) in PBS (pH 7.4)/1% BSA for 30 minutes at room temperature. Stained spheroplasts are twice washed with PBS/1% BSA and sorted on a FACS machine. Positively stained cells are grown in CAA/2% glucose medium with Carbenicillin (50 ng/ml)/Tetracycline (5 ng/ml) (e.g., Sigma) for 2 days at 37° C. A small amount (˜1 μl) of the liquid culture is plated onto a 100 mm CAA-glucose plate and incubated at 37° C. for 3 days.

Well isolated clones are picked from the plates with the positive cells recovered from the first and second round of selection. Individual CAA-RGD cultures are inoculated with the colonies and grown overnight at 30° C. Yeast cells from the overnight cultures are pelleted and used as templates in a Fc region specific PCR reaction (primers YD21 and YD32).

Positive clones recovered from the first round of selection will give a positive PCR signal for the presence of the Fc region. The validity of any PCR positive clones from the screen maybe further confirmed by immunostaining of spheroplasts with FITC conjugated anti-human Ig(Fc) antibody.

The method of screening described above may also be used, with small modifications, to screen a host cell library of the invention with the goal of isolating a cell displaying an antibody that is capable of binding an antigen (e.g., protein antigen). Briefly, a host cell library is incubated as described to allow for the cell surface display of antibodies or fragment thereof. Following incubation, the host cells are processed to generate spheroplasts. Spheroplasts are incubated with a labeled form of the antigen of interest (e.g., biotinylated antigen, FLAG-tagged antigen) to allow for specific binding of antigen to antibodies or a fragment thereof displayed on the plasma membrane of the spheroplasts. Subsequently, antigen bound spheroplasts are incubated with a fluorescently labeled secondary reagent (e.g., PE-streptavidin for biotinylated antigens, FITC conjugated anti-FLAG antibody for FLAG tagged antigens) that is capable of specific binding to the antigen. Spheroplasts with surface bound antigen/secondary reagent complexes are detected and isolated via fluorescently activated cell sorting. Vector DNA is recovered from the isolated cells. A second round of selection may be performed as described above. Isolated cells displaying an antibody with the desired antigen binding characteristics are processed as described above.

### 6.4 Example 4

### Yeast Plasma Membrane Displayed Naïve Human Antibody Library

The following sections describe a non-limiting example of a protocol that may be used for the generation of a yeast plasma membrane displayed naïve human antibody library.

6.4.1 cDNA Library Synthesis

First, total RNA is isolated from the peripheral blood mononuclear cells (PBMC) of several healthy donors e.g., by using QIAgen RNeasy kit. In addition, a pool of mRNA is obtained by combining material from several sources (e.g., Bioscience, Cat# 636170, BD Bioscience Cat. 6594-1, Origene technologies and Biochain Institute, Inc. Cat#M1234246). A human cDNA library is synthesized by using Superscript III RT kit (e.g., Invitrogen) following the manufacturer's instructions.

6.4.2 pYC2a-Heavy Chain Library Construction

Rearranged VH segments are PCR amplified from a human cDNA library (see 5.4.1). Primers which may be used are listed in Table 3. The heavy chain variable regions may be amplified from the cDNA library using Taq DNA polymerase (Invitrogen, cat. 18038-018), 50 μmol of yMedieu-VH1-15 and 50 μmol of the pooled reverse-Medieu-JH1, JH2 and JH3 primers as follows. After 5 minutes of denaturing, the template is amplified for 8 cycles at 95° C. for 30 sec, 52° C. for 60sec and 72° C. for 60 sec; the template is further amplified for 32 cycles at 95° C. for 30 sec, 62° C. for 30 sec, 72° C. for 60 sec and held at 72° C. for 7 minutes. The amplified VH fragments are agarose gel purified, resuspended in 20 μl TE pH 8.0 buffer and the concentration of each fragment determined. A mixture containing an equal amount of each VH fragment is digested with XhoI and SalI restriction endonucleases (New England Biolabs) and cloned into the similarly digested pYC2a-HC vector to create the VH library. The ligation product is phenol-chloroform extracted, precipitated and transformed into DH10B electrocompetent cells. Transformed cells are plated onto LB plates with 100 mg/ml Ampicillin and grown overnight at 37° C. 96 well isolated clones are sequenced to determine the library's degree of diversity. The library repertoire is recovered by flooding the plates with liquid medium and harvesting the cells. Library is frozen in small aliquots in 15% glycerol. 100 ml liquid culture medium with 100 μg/ml Ampicillin is inoculated with a small aliquot of the frozen library and incubated at 37° C. overnight. pYD-LC plasmid DNA comprising the naïve human heavy chain repertoire is recovered from the cells using a commercially available DNA purification kit (e.g. Qiagen DNA purification kit).

TABLE 3
Primers used for human naïve antibody library generation.

Human V heavy specific forward primers

| | | |
|---|---|---|
| yMedieu-VH1 | GTATCTCTCGAGAAAAGACAGGTKCAGCTGGTGCAGTCTGG | (SEQ ID NO: 68) |
| yMedieu-VH2 | GTATCTCTCGAGAAAAGACAGGTCCAGCTTGTGCAGTCTGG | (SEQ ID NO: 69) |
| yMedieu-VH3 | GTATCTCTCGAGAAAAGASAGGTCCAGCTGGTACAGTCTGG | (SEQ ID NO: 70) |
| yMedieu-VH4 | GTATCTCTCGAGAAAAGACARATGCAGCTGGTGCAGTCTGG | (SEQ ID NO: 71) |
| yMedieu-VH5 | GTATCTCTCGAGAAAAGACAGATCACCTTGAAGGAGTCTGG | (SEQ ID NO: 72) |
| yMedieu-VH6 | GTATCTCTCGAGAAAAGACAGGTCACCTTGAAGGAGTCTGG | (SEQ ID NO: 73) |
| yMedieu-VH7 | GTATCTCTCGAGAAAAGAGARGTGCAGCTGGTGGAGTCT | (SEQ ID NO: 74) |
| yMedieu-VH8 | GTATCTCTCGAGAAAAGACAGGTGCAGCTGGTGGAGTCTGG | (SEQ ID NO: 75) |
| yMedieu-VH9 | GTATCTCTCGAGAAAAGAGAGGTGCAGCTGTTGGAGTCTGG | (SEQ ID NO: 76) |
| yMedieu-VH10 | GTATCTCTCGAGAAAAGAGAGGTGCAGCTGGTGCAGWCYGG | (SEQ ID NO: 77) |
| yMedieu-VH11 | GTATCTCTCGAGAAAAGACAGSTGCAGCTGCAGGAGTCSGG | (SEQ ID NO: 78) |
| yMedieu-VH12 | GTATCTCTCGAGAAAAGACAGGTGCAGCTACAGCAGTGGGG | (SEQ ID NO: 79) |
| yMedieu-VH13 | GTATCTCTCGAGAAAAGAGARGTGCAGCTGGTGCAGTCTGG | (SEQ ID NO: 80) |
| yMedieu-VH14 | GTATCTCTCGAGAAAAGACAGGTACAGCTGCAGCAGTCAGG | (SEQ ID NO: 81) |
| yMedieu-VH15 | GTATCTCTCGAGAAAAGACAGGTGCAGCTGGTGCAATCTGG | (SEQ ID NO: 82) |

Human V heavy specific reverse primers

| | | |
|---|---|---|
| Medieu-JH1 | GAAGACGGATGGGCCCTTGGTCGACGCTGAGGAGACRGTGACCAGGGT | (SEQ ID NO: 83) |
| Medieu-JH2 | GAAGACGGATGGGCCCTTGGTCGACGCTGAAGAGACGGTGACCATTGT | (SEQ ID NO: 84) |
| Medieu-JH3 | GAAGACGGATGGGCCCTTGGTCGACGCTGAGGAGACGGTGACCGTGGT | (SEQ ID NO: 85) |

Human V kappa specific forward primers

| | | |
|---|---|---|
| yMedieu-Vκ1 | GTTATTGCTAGCGTTTTAGCARACATCCAGATGACCCAGTCTCC | (SEQ ID NO: 86) |
| yMedieu-Vκ2 | GTTATTGCTAGCGTTTTAGCAGMCATCCRGWTGACCCAGTCTCC | (SEQ ID NO: 87) |
| yMedieu-Vκ3 | GTTATTGCTAGCGTTTTAGCAGTCATCTGGATGACCCAGTCTCC | (SEQ ID NO: 88) |
| yMedieu-Vκ4 | GTTATTGCTAGCGTTTTAGCAGATATTGTGATGACCCAGACTCC | (SEQ ID NO: 89) |
| yMedieu-Vκ5 | GTTATTGCTAGCGTTTTAGCAGATRTTGTGATGACWCAGTCTCC | (SEQ ID NO: 90) |
| yMedieu-Vκ6 | GTTATTGCTAGCGTTTTAGCAGAAATTGTGTTGACRCAGTCTCC | (SEQ ID NO: 91) |
| yMedieu-Vκ7 | GTTATTGCTAGCGTTTTAGCAGAAATAGTGATGACGCAGTCTCC | (SEQ ID NO: 92) |
| yMedieu-Vκ8 | GTTATTGCTAGCGTTTTAGCAGAAATTGTAATGACACAGTCTCC | (SEQ ID NO: 93) |
| yMedieu-Vκ9 | GTTATTGCTAGCGTTTTAGCAGACATCGTGATGACCCAGTCTCC | (SEQ ID N0: 94) |
| yMedieu-Vκ10 | GTTATTGCTAGCGTTTTAGCAGAAACGACACTCACGCAGTCTCC | (SEQ ID NO: 95) |
| yMedieu-Vκ11 | GTTATTGCTAGCGTTTTAGCAGAAATTGTGCTGACTCAGTCTCC | (SEQ ID NO: 96) |

Human V kappa specific reverse primers

| | | |
|---|---|---|
| Ckappa | GCATGCTCGACATCGATTCACTAACACTCTCCCCTGTTGAAGCTC | (SEQ ID NO: 97) |

Human V lambda specific forward primers

| | | |
|---|---|---|
| yMedieu-Vλ1 | GTTATTGCTAGCGTTTTAGCACAGTCTGTGCTGACTCAGCCACC | (SEQ ID NO: 98) |
| yMedieu-Vλ2 | GTTATTGCTAGCGTTTTAGCACAGTCTGTGYTGACGCAGCCGCC | (SEQ ID NO: 99) |
| yMedieu-Vλ3 | GTTATTGCTAGCGTTTTAGCACAGTCTGCCCTGACTCAGCCT | (SEQ ID NO: 100) |
| yMedieu-Vλ4 | GTTATTGCTAGCGTTTTAGCATCCTATGWGCTGACWCAGCCA | (SEQ ID NO: 101) |
| yMedieu-Vλ5 | GTTATTGCTAGCGTTTTAGCATCCTATGAGCTGACACAGCTACC | (SEQ ID NO: 102) |
| yMedieu-Vλ6 | GTTATTGCTAGCGTTTTAGCATCTTCTGAGCTGACTCAGGACC | (SEQ ID NO: 103) |
| yMedieu-Vλ7 | GTTATTGCTAGCGTTTTAGCATCCTATGAGCTGATGCAGCCAC | (SEQ ID NO: 104) |
| yMedieu-Vλ8 | GTTATTGCTAGCGTTTTAGCACAGCYTGTGCTGACTCAATC | (SEQ ID NO: 105) |
| yMedieu-Vλ9 | GTTATTGCTAGCGTTTTAGCACWGSCTGTGCTGACTCAGCC | (SEQ ID NO: 106) |
| yMedieu-Vλ10 | GTTATTGCTAGCGTTTTAGCAAATTTTATGCTGACTCAGCCCCA | (SEQ ID NO: 107) |
| yMedieu-Vλ11 | GTTATTGCTAGCGTTTTAGCACAGRCTGTGGTGACYCAGGAGCC | (SEQ ID NO: 108) |
| yMedieu-Vλ12 | GTTATTGCTAGCGTTTTAGCACAGGCAGGGCTGACTCAGCCACC | (SEQ ID NO: 109) |

Human Vlambda specific reverse primers

| | | |
|---|---|---|
| Clambda1 | GCATGCTCGACATCGATTCACTATGAACATTCTGTAGGGGCCACTG | (SEQ ID NO: 110) |
| Clambda2 | GCATGCTCGACATCGATTCACTAAGAGCATTCTGCAGGGGCCACTG | (SEQ ID NO: 111) |

6.4.3 pYD-LC Library Construction

Rearranged VL segments are PCR amplified from a human cDNA library (see 5.4.1). Primers which may be used are listed in Table 3. Twelve yMedieu-VX forward primers are paired with two λ reverse primers to amplify the antibody λ light chain variable and constant regions. Similarly, eleven yMedieu-yVK forward primers are paired with the κ reverse primer to amplify the antibody κ light chain variable and constant regions. Using Pfu Ultra (Stratagene), each reaction is done separately using 10 μmol of each primer as follows: after the initial 3 minutes denaturation, the PCR reaction is amplified for 30 cycles at 95° C. for 30 sec, 52° C. for 30 sec, 68° C. for 90 sec and held at 68° C. for 10 minutes. Equal amounts of the PCR products are pooled, agarose gel purified and digested with NheI and ClaI restriction endonucleases. Using similarly digested pYD-LC vector, the products are T4 DNA ligated, phenol-chloroform extracted, precipitated and transformed into DH10B electrocompetent cells. Transformed cells are plated onto LB plates with 100 μg/ml Ampicillin and grown overnight at 37° C. 96 well isolated clones are sequenced to determine the library's degree of diversity. The library repertoire is recovered by flooding the plates with liquid medium and harvesting the cells. Library is frozen in small aliquots in 15% glycerol. A small aliquot of frozen cells is used to inoculate 100 ml liquid culture with 100 μg/ml Ampicillin and incubated at 37° C. overnight. pYD-LC plasmid DNA comprising the naïve human light chain repertoire is recovered from the cells using a commercially available DNA purification kit (e.g. Qiagen DNA purification kit).

6.4.4 Generation of a Yeast Cell Population Comprising the Plasma Membrane Displayed Naïve Human Antibody Library

A yeast cell population comprising a library of plasma membrane displayed naïve human antibodies may be generated by transforming a suitable host cell (e.g., LB 3003-4B (MATalpha mnn9 ur3 leu2 his4) cells (ATCC)) with both the naïve human light and heavy chains. Transformation may be performed using a protocol developed for library scale transformation (e.g., DSY kit from Dualsystems Biotech). The yeast cell population comprising a library of plasma membrane displayed naïve human antibodies may then be used in conjunction with a screening method described herein.

Alternatively, a yeast cell population comprising a library of plasma membrane displayed naïve human heavy and light chains may also be generated using the methods described in US Patent publication 2003/0186374 to Hufton. According to this method, a haploid yeast cell population comprising an expression library of antibody heavy chains is mated with a haploid yeast population of the opposite mating type comprising an expression library of antibody light chains to generate a population of diploid yeast cells comprising an expression library of full length antibodies. An outline of the protocol is provided below.

The pYD-HC plasmid DNA preparation comprising the naïve human heavy chain repertoire is used to transform a haploid yeast strain (e.g., EGY48 (MATa, ura3, his3, trp1, LexAₒₚ₍ₓ₆₎-LEU2) from Clontech). Transformation is performed using a protocol developed for library scale transformation (e.g., DSY kit from Dualsystems Biotech). Transformed cells are elected on -Ura plates. The pYD-LC plasmid DNA preparation comprising the naïve human light chain repertoire is used to transform a haploid yeast strain of the opposite mating type (e.g., YM4271 (MATa, ura3, his3-200, lys2-801, ade2-101, ade5, trp1-901, leu2-3,112, tyr1-501,gal4Δ, gal80Δ, ade5::hisG) from Clontech). Transformation is performed using a protocol developed for library scale transformation (e.g., DSY kit from Dualsystems Biotech). Transformed cells are selected on -Trp plates. Transformation is performed using a protocol developed for library scale transformation (e.g., DSY kit from Dualsystems Biotech). pYD-HC and pYD-LC comprising haploid cells are mixed and plated onto -Trp, -Ura plates to select for diploid cells comprising both a heavy and light chain display vector. The diploid yeast cell population comprising a library of plasma membrane displayed naïve human antibodies may be used in conjunction with a screening method described herein (see Examples 1 and 2).

### 6.5 Example 5

### Methods of Screening a 2μ-scFv-TM Plasmid Based S. cerevisiae Library

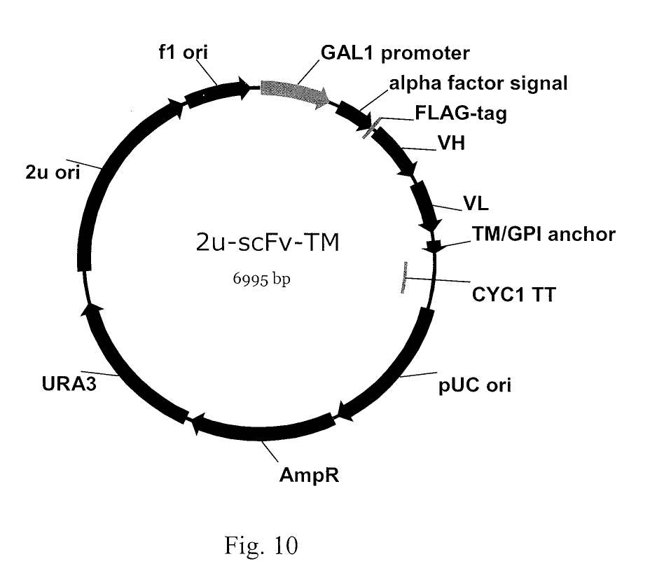

2μ-scFv-TM plasmid construction: A polynucleotide fragment encoding the antibody fragment fused to the transmembrane anchor domain was PCR amplified from the pYC-scFv-TM vector using the HinAlphaF (SEQ ID NO:112) and CycR (SEQ ID NO:113) primers. The amplified polynucleotide fragment was digested with the HindIII and NotI restriction endonucleases and ligated into a HindIII/NotI digested pYes2_CT (Invitrogen) vector. The resulting 2μ-scFv-TM vector (FIG. 10) was recovered using standard laboratory techniques.

Fluorescent labeling of spheroplasts: A single yeast colony was inoculated into SDCAA growth medium and grown at 30° C. overnight to reach OD600 nm of 1-3. The yeast cells were pelleted and resuspended in induction medium SGCAA and grown overnight at 20° C. to induce antibody expression. Spheroplasts were generated by washing approximately 10⁸ yeast cells twice with 1 mL SE buffer (1M sorbitol, 100 mM EDTA in H2O), resuspending them in 1 mL lyticase digestion buffer containing 100 Units/ml lyticase (SigmaAldrich), and incubating the resuspended yeast cells at 30° C. for 30 minutes. Spheroplasts were collected by centrifugation at 500g for 4 minutes and washed twice with 0.5 ml PBSA (1% BSA in PBS) to remove any lyticase. For dual detection by flow cytometry, spheroplasts were resuspended in 0.5 mL PBSA containing 10 μg/ml anti-FLAG M2 antibody and 25 μg/ml biotinylated antigen (e.g., EphA4-Fc-biotin or EphA2-Fc-Biotin). Spheroplasts were stained at 4° C. for 1 hour and washed twice with 0.5 ml PBSA. Secondary staining was performed by incubating the cells with goat anti mouse IgG-FITC conjugate (Molecular Probes) and streptavidin-allophycocyanin (Caltag) at 4° C. for 30 minutes. Spheroplasts were washed twice with 0.5 ml PBSA and resuspend in 0.5 ml PBSA for flow cytometry. The anti-FLAG/anti IgG-FITC FITC antibody staining visualizes the presence of a cell surface displayed FLAG-tagged antibody. Positive staining for biotinylated antigen/strepavidin-allophycocyanin on the other hand demonstrates that the cell surfaced displayed antibody is capable of specifically bind its target antigen.

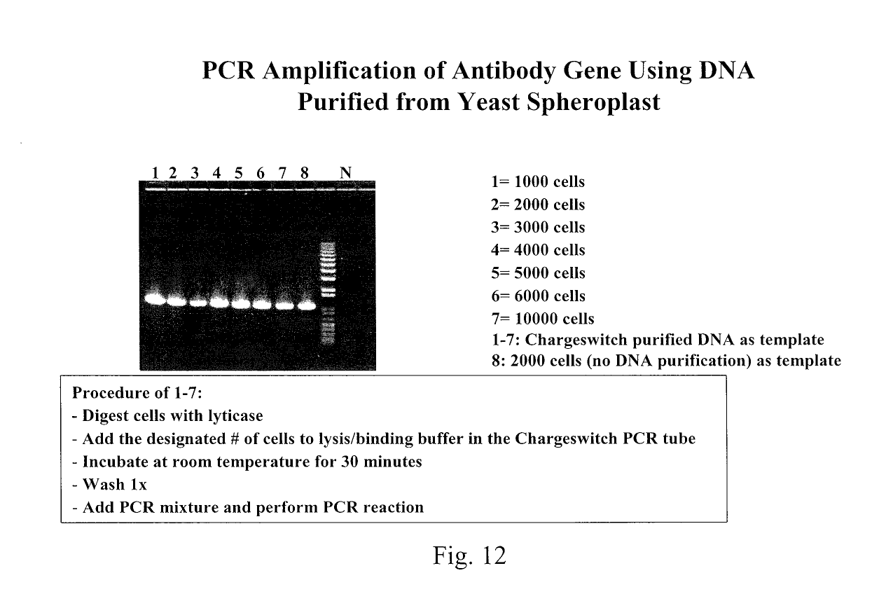

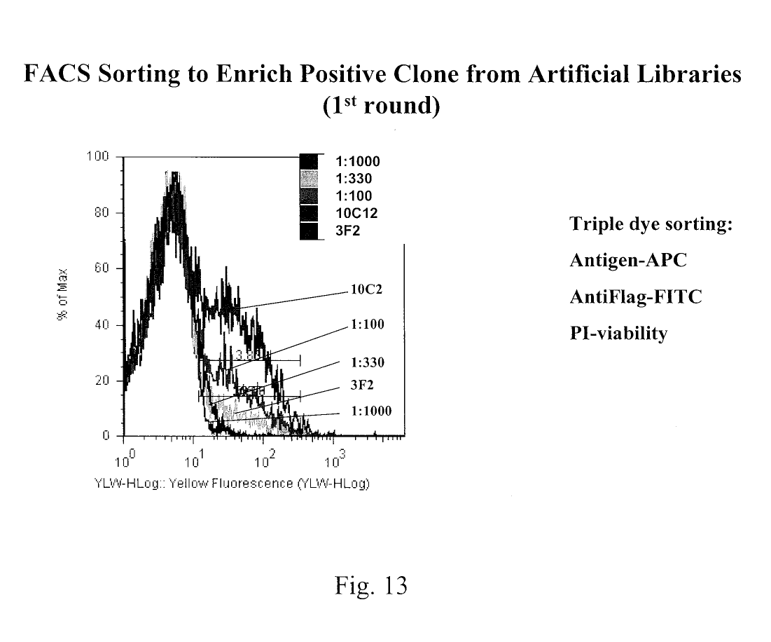

FACS sorting and antibody gene amplification: A single drop of propidium iodide (PI) solution was added to the cell suspension prior to FACS. Gates were set to only analyze the PI negative spheroplasts. Sorting gates were set to only include FITC and allophycocyanin double positive cells. The double positives accounted for approximately 0.1% of the PI negative spheroplasts (see, for example, FIG. 13).The double positive cells were collected into Chargeswitch EasyPlex™ gDNA PCR tubes (Invitrogen) containing 100 μl lysis/binding buffer. Tubes were incubated at room temperature for 30 minutes. Reaction tubes were washed twice with 120 μl wash buffer. DNA was recovered following the manufacturer's protocol. Polynucleotide fragments encoding the antibody and the transmembrane domain were amplified using the AlphaFC (SEQ ID NO:114)/His6R (SEQ ID NO:115) and His6F (SEQ ID NO:116)/HcRev (SEQ ID NO:117) primer pairs, respectively. Data presented in FIG. 12 demonstrates the robustness of the PCR reactions used. The two fragments were gel purified and assembled in a single PCR reaction using the AlphaFD (SEQ ID NO:118) and HTMRD (SEQ ID NO:119) primers. The single PCR product was purified and combined with AflII/XhoI digested 2μ-scFv-TM vector at a 10 to 1 mass ratio. The PCR fragment/vector mix was precipitated with ethanol and resuspended in 10 μl of H₂O for gap repair/yeast transformation.

Yeast transformation by gap repair: The yeast transformation/gap repair was performed substantially as described by Swers J S et al. Biochem Biophys Res Commun. 350(3):508-13 (2006). Briefly, a single colony of the BJ5457 yeast strain was grown in YPD medium overnight at 30° C. The overnight cell culture was diluted into 50 ml of fresh YPD to reach a final OD_{600 nm} of 0.1 and grown for about 5 hours to log phase (OD_{600 nm}=1.2˜1.5). Cells were harvested by centrifugation, resuspended in 50 ml of freshly prepared 10 mM Tris pH 8.0, 25 mM dithiothreitol (DTT) in YPD, and shaken for 20 min at 30° C. Cells were washed once with 15 ml of buffer E (10 mM Tris pH 7.5, 270 mM sucrose, 1 mM MgCl₂) and resuspended in about 150 μl buffer E at a density of 2×10⁸ cells per 50 μl aliquot of electrocompetent cells. A aliquot of the gap repair DNA mix comprising 1 μg of Afl II/Xho I digested 2μ-scFv-TM vector and 10 μg of PCR product was added to 50 μl of electrocompetent cells and incubated on ice for 5 minutes. The yeast cells were electroporated using a 0.2 cm cuvette at 0.54 kV/25 μF, transferred into 1 ml of YPD, and incubated for 1 hour at 30° C. The electroporated cells were collected by centrifugation, plated onto SDCAA plates (-URA, +TRP) and incubated at 30° C. overnight.

6.5.1 Proof-of-Principle Screen of a 2β-scFv-TM Plasmid Based Artificial S. cerevisiae Library.

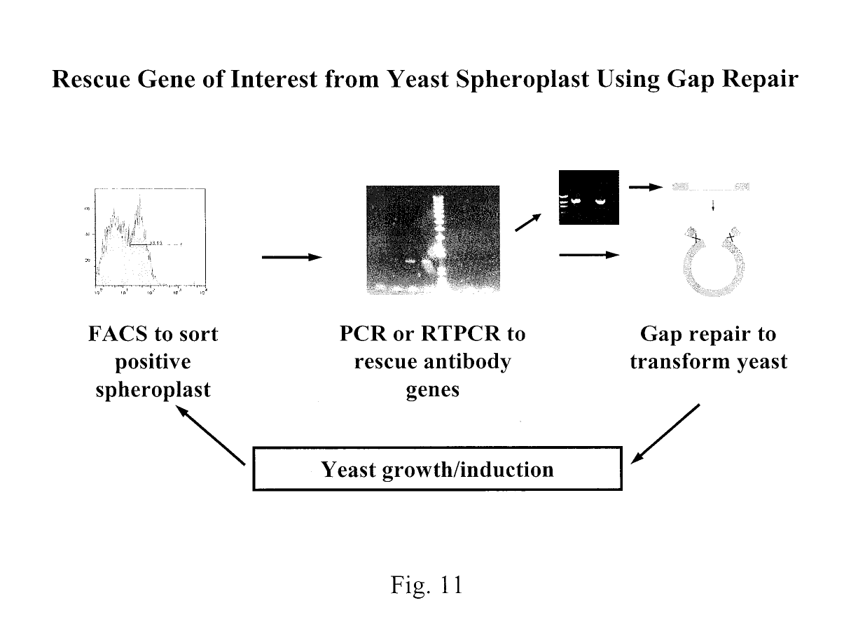

The following section describes a non-limiting example of a method for screening and isolating a desired full antibody from a 2μ-scFv-TM plasmid based artificial S. cerevisiae plasma membrane displayed antibody library. The screen utilizes successive rounds comprising the steps of (1) selection of spheroplast s displaying on their surface an antibody with desired characteristics (e.g., binding to a specific antigen); (2) amplification of antibody encoding polynucleotides from the selected cells; (3) generation of a secondary library of cells expressing the antibodies selected in step 1 (FIG. 11).

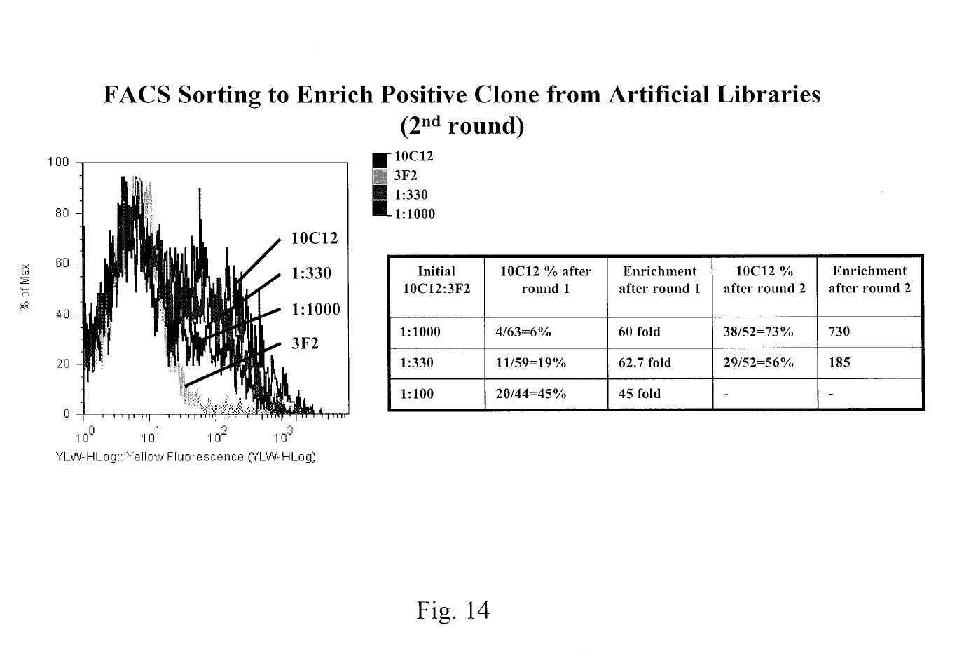

The screen was performed on several artificial libraries generated by mixing yeast cells expressing the 10C12 anti-EphA4 and 3F2 anti-EphA2 TM anchored antibody fragment at ratios of 1:100, 1:330, and 1:1000. Spheroplasts were stained with anti-FLAG/anti IgG-FITC antibodies and biotinylated EphA2/streptavidin-allophycocyanin. Fluorescent intensity profile of the various cell populations in the first round of selection are shown in FIG. 13. The experiment included pure populations of 10C12 and 3F2 antibody displaying cells. Double positive cells were selected in the first round and used to prepare a secondary library according to the methods described above. Fluorescent intensity profile of the cell population representing the various secondary libraries is shown in FIG. 14. The frequency of double positive cells is significantly increased in each of the secondary libraries over the frequency seen in the corresponding primary libraries.

The efficiency of the selection process was evaluated by analyzing ˜50 randomly selected clones comprising antibody genes amplified from the double positive cell population of the first and second round of selection. The results are presented in FIG. 14. 4/63 (6%) and 38/52 (73%) of the random clones representing the double positive cells isolated from the 1:1000 library in the first and second, respectively, round of selection contained the 10C12 antibody gene. This represents a 60-fold and 730-fold total enrichment after the first and second, respectively, round of selection. 11/59 (19%) and 29/52 (56%) of the random clones representing the double positive cells isolated from the 1:330 library in the first and second, respectively, round of selection contained the 10C12 antibody gene. This represents a 63-fold and 185-fold total enrichment after the first and second, respectively, round of selection. 20/44 (45%) of the random clones representing the double positive cells isolated from the 1:100 library in the first round of selection contained the 10C12 antibody gene. This represents a 45-fold enrichment. The double positive cells isolated in the first round form the 1:100 library were not processed in the second selection round.

6.6 Methods Related to Cell Surface Display of Antibodies in P. pastoris.

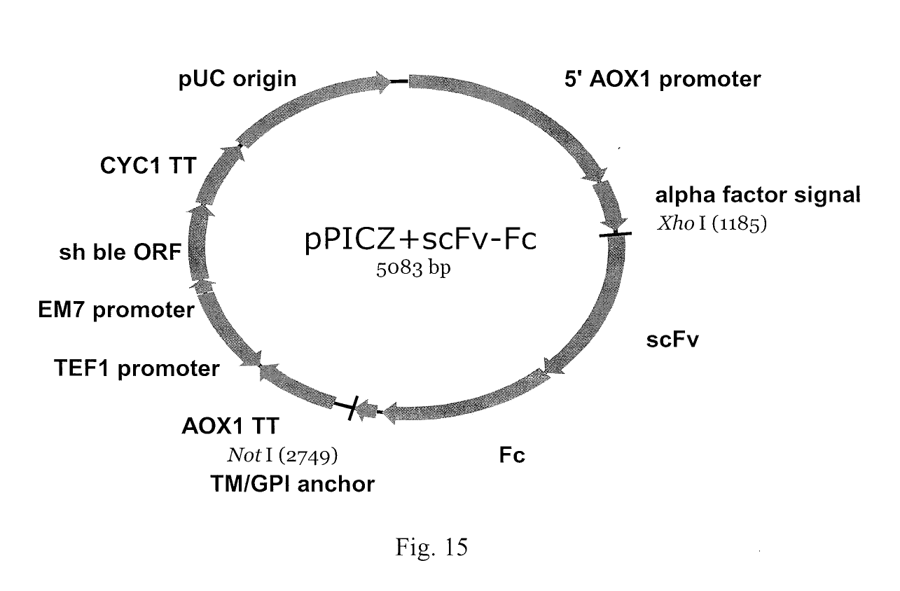

Vectors and Strains: Pichia pastoris host strain X-33 and plasmid pPICZα were obtained from Invitrogen. GPI-signal and transmembrane domain anchored scFv and scFv-Fc antibody fragments were excised from S. cerevisiae plasmid constructs described herein and cloned into Xho I/Not I digested vector pPICα. Cloning procedures were performed according to standard protocols. A schematic representation of the pPICZ+scFv-Fc vector is shown in FIG. 15 as a representative example of the vectors generated.

For full length antibody (IgG) display, polynucleotide fragments encoding the antibody heavy and light chains were separately cloned into pPICZα vector to generate the pPICZα-Hc and pPICZα-Lc constructs, respectively. A recognition site for the Pme I restriction endonuclease was removed from the AOX1 promoter region of the pPICZα-Hc plasmid using the QuickChange Kit (Stratagene) and the PmeIF (SEQ ID NO:120)/PmeIR (SEQ ID NO:121) primers. The light chain expression cassette of the pPICZα-Lc vector was isolated as a BglII/BamHI fragment and cloned into a BglII cleaved pPICZα-Hc vector to generate the 10C12 P. pastoris full length antibody display vector.

An episomal antibody expression vector was generated by inserting the PARS1 autonomous replication sequence into the PICZa10C12ScFvFc-GPI and PICZa10C12ScFvHF-GPI vectors. A PARS1 comprising polynucleotide sequence was amplified in a single reaction using the PARS1F1 (SEQ ID NO:122), PARS1F2 (SEQ ID NO:123), PARS1R1 (SEQ ID NO:124), PARS1R2 (SEQ ID NO:125) primers. The PciI restriction endonuclease digested PCR product was cloned into the PciI digested vectors. Yeast transformation were performed according to standard protocols.

Pichia Transformation: Antibody display vectors were linearized by Pme I digestion and electroporated into X-33 Pichia pastoris electrocompetent cells. Transformed Pichia pastoris cells comprising an integrated antibody gene were selected for growth on YPD (1% yeast extract, 2% tryptone, 2% dextrose) plates supplemented with 100 ug/ml of Zeocin. Episomal vector transformation was performed according to the same protocol with the exception that the episomal vector was not linearized prior to transformation.

Antibody expression: Single colonies of transformed X-33 cells were grown overnight at 30° C. in 5 ml of BMGY (1% yeast extract, 2% peptone, 100 mM potassium phosphate, pH6.0, 1.34% YNB, 4×10⁻⁵% biotin, 1% glycerol) in the presence of Zeocin (100 ug/ml). Cells were pelleted and resuspended in methanol containing BMYY (1% yeast extract, 2% peptone, 100 mM potassium phosphate, pH6.0, 1.34% YNB, 4×10⁻⁵% biotin, 0.5% methanol) medium at OD600 of 1.0 to induce antibody expression for 1 or 2 days. Induction was performed according to the manufacturer's protocol (Invitrogen).

Spheroplasts Preparation: Following induction, ˜10⁸ yeast cells were washed twice with sterile water, once with 1 ml of fresh SED (50 mM DTT in 1M sorbitol), and once with 1M sorbitol. Cells were resuspended in 100 ul of SEC buffer (1M sorbitol, 1 mM EDTA and 10 mM sodium citrate buffer, pH 5.8) containing 2.5 units of Zymolyase and incubated for 2 min at room temperature to generate spheroplast. The spheroplasts were washed once with 1M sorbitol and once with 1% BSA in PBS.

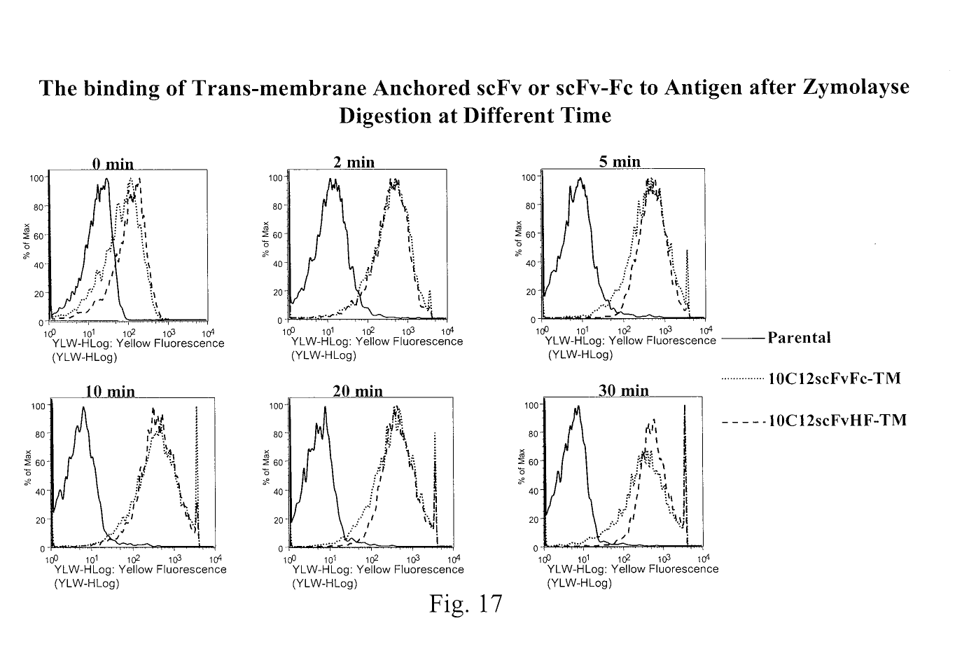

Flow cytometry of P. pastoris spheroplasts: The effect of extended zymolase digestion on antigen binding by trans-membrane domain anchored 10C12 anti-EphA4 His/FLAG-tagged-scFv (10C12ScFvHF-TM) or scFv-Fc (10C12ScFvFc-TM) molecules expressed in P. pastoris was examined. P. pastoris cells comprising various plasma membrane displayed antibody expression vectors were induced following standard protocols. Spheroplasts were prepared as described above. Zymolase digestion was allowed to proceed for 0 min, 2 min, 5 min, 10 min, 20 min or 30 min. Spheroplasts were stained with EphA4-Fc-biotin/streptavidin-PE and analysed on a flow cytometer. Fluorescent intensity profile of the stained spheroplasts is shown in FIG. 17. Similarly stained parental P. pastoris spheroplasts were included as negative control. The fluorescent intensity profile of TM anchored scFv and scFv-Fc expressing cells is almost identical at all zymolase digestion time points tested. The separation between the mean fluorescent intensity (MFI) of antibody expressing and control cells reaches a 2 log maximum after 2 minutes of zymolase digestion. Further incubation with zymolase does not appear to increase staining intensity of the spheroplasts.

Flow cytometry of P. pastoris cells: Approximately 5 million cells expressing trans-membrane anchored 10C12 anti-EphA4 scFv or scFv-Fc were washed with buffer (1% BSA/PBS) twice and resuspended in 100 ul buffer. Antigen binding of cell surface displayed antibodies were detected by EphA4-Fc-biotin (1 ug/ml)/streptavidin-PE staining Anti-hIgGFc-FITC was used to detect human Fc on cell surface displayed human Fc domains. Staining and flow cytometry was performed as described above.

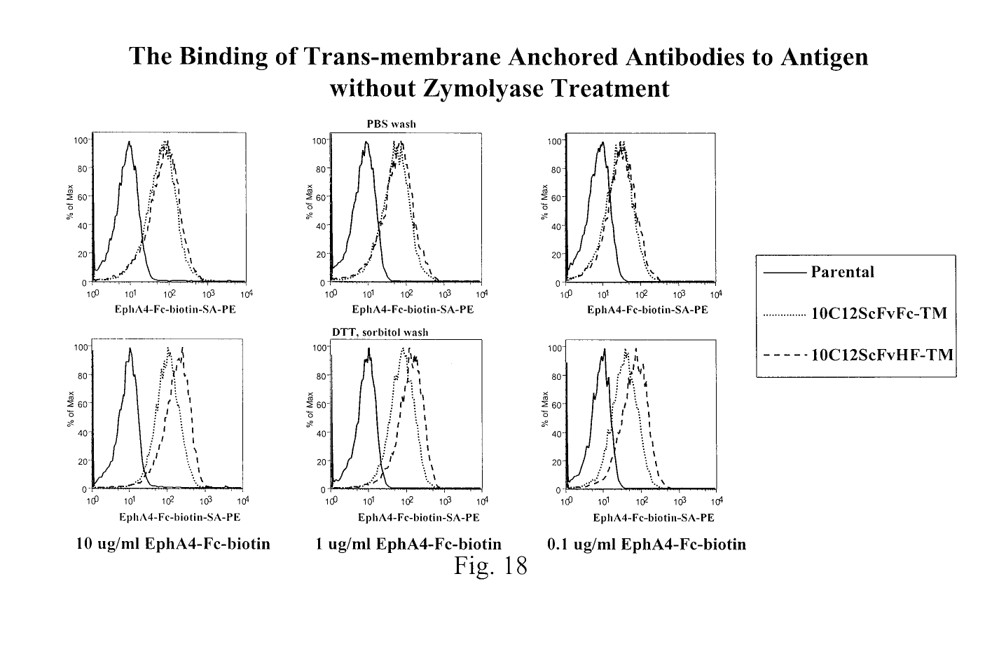

Accessibility of the P. pastoris expressed TM anchored 10C12 anti-EphA4 scFv or scFv-Fc molecules to antigen in intact cells was assessed. P. pastoris cells comprising an integrated plasma membrane displayed antibody expression vector were induced following standard protocols. Approximately 5 million cells were washed either with PBS or 50 mM DTT/1 M sorbitol. Following the wash, cells were incubated with 10 μg/ml, 1 μg/ml or 0.1 μg/ml EphA4-Fc-biotin, stained with streptavidin-PE and analyzed on a flow cytometer. Similarly stained parental P. pastoris cells were included as negative control. Results are shown in FIG. 18. The fluorescent intensity profile of PBS washed TM anchored scFv and scFv-Fc expressing cells are identical at each concentration of EphA4-Fc-biotin tested; the separation between the MFI of antibody expressing cells and parental cells is not dependent on antigen concentration within the range tested. The fluorescent intensity profile of DTT/sorbitol washed TM anchored scFv-Fc expressing cells is higher than that of the scFv expressing cells at all EphAn-Fc-biotin concentrations tested; the separation between the MFI of antibody expressing cells and parental cells is not dependent on antigen concentration within the range tested.

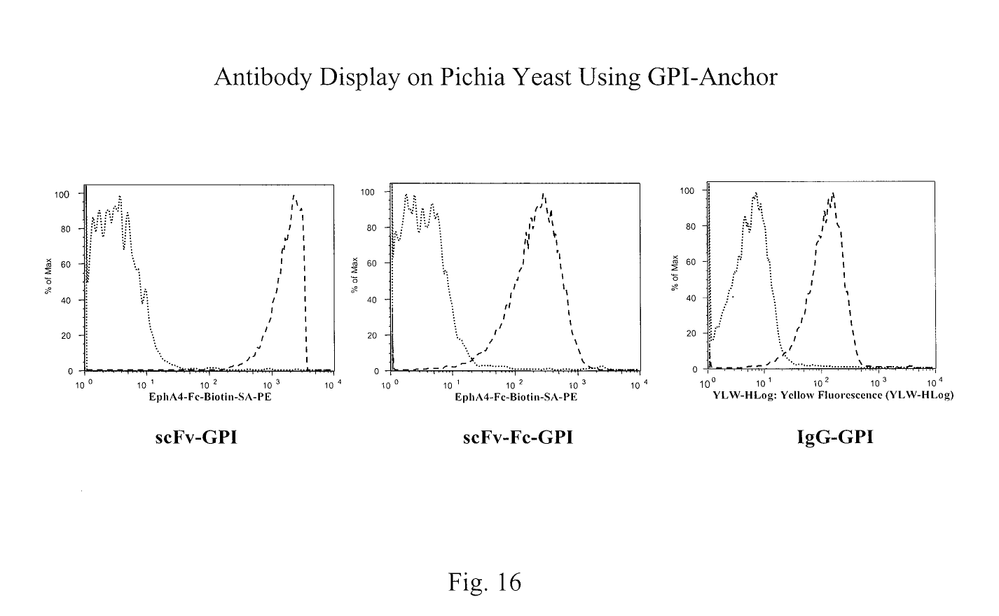

MFI of P. pastoris cells comprising GPI anchored antibodies is shown in FIG. 16. P. pastoris cells comprising various GPI anchored plasma membrane displayed antibody expression vectors were induced following standard protocols. Induced cells were stained with EphA4-Fc-biotin/streptavidin-PE and analysed on a flow cytometer as described above.

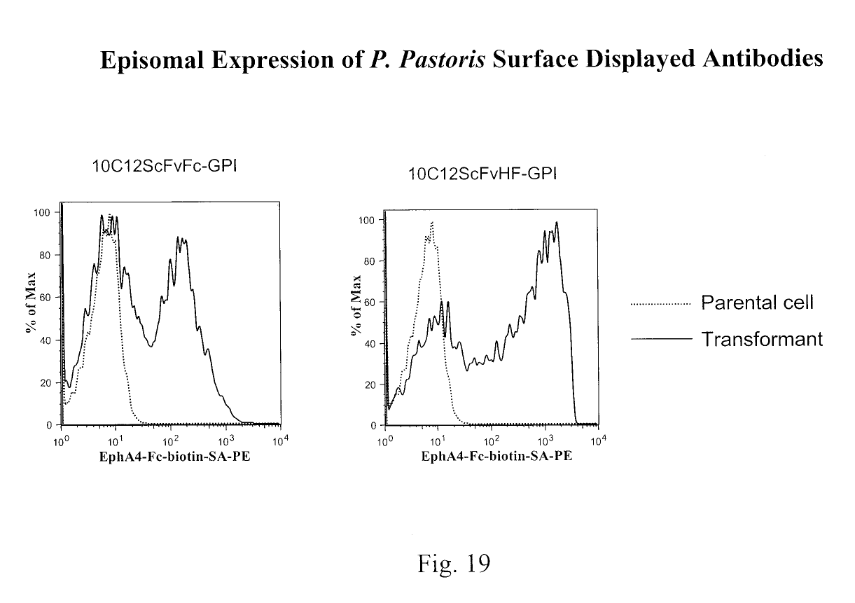

Episomal expression of cell surface displayed antibodies in P. pastoris. P. pastoris cells comprising an episomal surface displayed antibody expression vector were induced following standard protocols. Approximately 5 million cells were washed with PBS or 50 mM DTT/1 M sorbitol. Following the wash, cells were incubated with 10 μg/ml EphA4-Fc-biotin, stained with streptavidin-PE and analyzed on a flow cytometer. Similarly stained parental P. pastoris cells were included as negative control. Results are shown in FIG. 19. The MFI of 10C12ScFvFc-GPI and 10C12ScFvHF-GPI expressing cells were 1.5-2 logs higher than that of the parental cells.

Whereas, particular embodiments of the invention have been described above for purposes of description, it will be appreciated by those skilled in the art that numerous variations of the details may be made without departing from the invention as described in the appended claims.

All publications, patents and patent applications mentioned in this specification are herein incorporated by reference into the specification to the same extent as if each individual publication, patent or patent application was specifically and individually indicated to be incorporated herein by reference. In addition, U.S. Provisional Application No. 60/889,019, filed Feb. 9, 2007, is incorporated by reference in its entirety for all purposes.

1-44. (canceled)

45. A library comprising polynucleotides encoding a heterogeneous population of antibodies that may be displayed on the extracellular surface of the plasma membrane of a yeast cell, wherein said heterogeneous population of antibodies comprise a library of heavy chain variable region sequences and/or a library of light chain variable region sequences.

46. The library of claim 45, wherein said antibodies are murine antibodies, chimeric antibodies, humanized antibodies, human antibodies or synthetic antibodies.

47. The library of claim 45, wherein said heterogeneous population of antibodies are antigen binding fragments selected from the group consisting of a single-chain Fv (scFv); an Fab fragment; an F(ab′) fragment; and an Fd fragment.

48. The library of claim 47, wherein said antigen binding fragments are each fused to an Fc region.

49. The library of claim 45, wherein said antibodies comprise an amino acid sequence that targets said antibodies to the cell surface, wherein said amino acid sequence is fused to the C-terminal end of the heavy chain or the C-terminal end of the light chain.

50. The library of claim 49, wherein said amino acid sequence that targets said antibodies to the cell surface comprises a transmembrane domain or a GPI anchor domain.

51. The library of claim 50, wherein said transmembrane domain comprises an amino acid sequence set forth as SEQ ID NO:2, 4, or 6.

52. The library of claim 47, wherein said antibodies comprise an amino acid sequence that targets said antibodies to the cell surface, wherein said amino acid sequence is fused to the C-terminal end of the heavy chain or the C-terminal end of the light chain.

53. The library of claim 52, wherein said amino acid sequence that targets said antibodies to the cell surface comprises a transmembrane domain or a GPI anchor domain.

54. The library of claim 53, wherein said transmembrane domain comprises an amino acid sequence set forth as SEQ ID NO:2, 4, or 6.

55. The library of claim 48, wherein said antibodies comprise an amino acid sequence that targets said antibodies to the cell surface, wherein said amino acid sequence is fused to the C-terminal end of the Fc region.

56. The library of claim 55, wherein said amino acid sequence that targets said antibodies to the cell surface comprises a transmembrane domain or a GPI anchor domain.

57. The library of claim 56, wherein said transmembrane domain comprises an amino acid sequence set forth as SEQ ID NO:2, 4, or 6.

58. A method of displaying a population of antibodies on the extracellular surface of the plasma membrane of a population of yeast cells comprising
a) transforming a population of yeast cells with the library of claim 45; and
b) culturing said population of yeast cells under conditions that allow display of an antibody on the extracellular surface of the plasma membrane of said population of yeast cells.

59. A method of isolating an antibody having a desirable binding characteristic comprising:
a) culturing a population of yeast cells comprising a library of claim 45 under conditions that allow display of an antibody on the extracellular surface of the plasma membrane of said population of yeast cells;
b) contacting said yeast cells with an antibody ligand; and
c) sorting said yeast cells based on the binding of said antibody ligand thereby isolating at least one cell expressing an antibody having the desired binding characteristic;

60. The method of claim 59 wherein said desirable binding characteristic is:
a) binding to a specific antigen;
b) increased binding to a specific antigen;
c) decreased binding to a specific antigen;
d) binding to an effector molecule selected from the group consisting of C1q, FcγRI, FcγRII and FcγRIIIA;
e) increased binding to an effector molecule selected from the group consisting of C1q, FcγRI, FcγRII and FcγRIIIA; or
f) decreased binding to an effector molecule selected from the group consisting of C1q, FcγRI, FcγRII and FcγRIIIA.

61. The method of claim 59, wherein said population of yeast cells comprise a genetic mutation rendering the cell wall sufficiently porous to make it permeable for a protein antigen or an antibody or a fragment thereof.

62. The method of claim 61, wherein said population of yeast cells comprise a genetic mutation in mnn9 or an orthologue of mnn9.

63. The method of claim 59, further comprising:
a) contacting said yeast cells with an enzyme that renders the cell wall sufficiently porous to make it permeable for an antibody ligand.

64. The method of claim 59, wherein said population of yeast cells are selected from the group consisting of: Saccharomyces cerevisiae, Hansenula polymorpha, Kluyveromyces lactis, Pichia pastoris, Schizosaccharomyces pombe and Yarrowia lipolytica.
